(19) United States
(12) Patent Application Publication
(10) Pub. No.: US 2026/0184762 A1
(43) Pub. Date: Jul. 2, 2026

(54) T-Cell Modulatory Polypeptides and Methods of Use Thereof

(71) Applicant: Cue Biopharma, Inc., Boston, MA (US)

(72) Inventors: Anish Suri, Boston, MA (US); Chee Meng Low, Boston, MA (US)

(21) Appl. No.: 19/568,309

(22) Filed: Mar. 16, 2026

Related U.S. Application Data
(63) Continuation of application No. PCT/US2024/047548, filed on Sep. 19, 2024.
(60) Provisional application No. 63/539,222, filed on Sep. 19, 2023.

(51) Int. Cl. C07K 14/74; A61K 38/00; C07K 7/04; C07K 14/47; C07K 14/55; C07K 14/705; C07K 16/30; C07K 19/00; C12N 15/62; C12N 15/63

(52) U.S. Cl. CPC C07K 14/70539; A61K 38/00; C07K 7/04; C07K 14/4726; C07K 14/55; C07K 14/7056; C07K 16/30; C07K 19/00; C12N 15/62; C12N 15/63; C07K 2317/52; C07K 2317/569; C07K 2317/622; C07K 2317/71; C07K 2319/00

(57) ABSTRACT

The present disclosure provides a T-cell modulatory multimeric polypeptide (TMMP) comprising at least one heterodimer, where the heterodimer comprises first and second polypeptide chains and where the heterodimer includes an NKG2 peptide, an HLA-E heavy chain polypeptide, a beta-2 microglobulin polypeptide, and an immunoglobulin Fc polypeptide. The heterodimer can also include an immunomodulatory polypeptide and/or a cancer-targeting polypeptide. The TMMPs are useful in methods of modulating an immune response, which methods are also provided.

### CROSS-REFERENCE TO RELATED APPLICATIONS

This application is a continuation of PCT Application No. PCT/US2024/047548, filed Sep. 19, 2024, which claims the benefit of U.S. Provisional Patent Application No. 63/539,222, filed Sep. 19, 2023, which applications are incorporated herein by reference in their entirety.

### INCORPORATION BY REFERENCE OF SEQUENCE LISTING PROVIDED AS A SEQUENCE LISTING XML FILE

A Sequence Listing is provided herewith as a Sequence Listing XML, “CUEB-156WO_SEQLIST”, created on Oct. 16, 2024 and having a size of 159,751 bytes. The contents of the Sequence Listing XML are incorporated herein by reference.

### INTRODUCTION

Natural killer (NK) cells are cytotoxic lymphocytes that are CD56-positive (CD56⁺) and CD3-negative (CD3⁻). NK cells can kill multiple adjacent cells if these cells display surface markers associated with oncogenic transformation. NK cells are thus of interest in cancer therapy. Unlike T cells, NK cells do not express a T-cell receptor (TCR). NK cells may express activating CD94/NKG2C receptors and/or inhibitory CD94/NKG2A receptors. Activating CD94/NKG2C receptors and inhibitory CD94/NKG2A receptors can bind to a peptide-major histocompatibility complex (pMHC), comprising a peptide, an HLA-E heavy chain polypeptide, and a beta-2 microglobulin (β2M) polypeptide SUMMARY

The present disclosure provides a T-cell modulatory multimeric polypeptide (TMMP) comprising at least one heterodimer, where the heterodimer comprises first and second polypeptide chains and where the heterodimer includes an NKG2 peptide, an HLA-E heavy chain polypeptide, a beta-2 microglobulin polypeptide, and an immunoglobulin Fc polypeptide. The heterodimer can also include an immunomodulatory polypeptide and/or a cancer-targeting polypeptide. The TMMPs are useful in methods of modulating an immune response, which methods are also provided.

### BRIEF DESCRIPTION OF THE DRAWINGS

FIGS. 1A-1G provide amino acid sequences of wild-type (FIG. 1A; SEQ ID NO: 1) HLA-E heavy chains and variants (FIG. 1B-1G; SEQ ID NOs:2-7, respectively).

FIGS. 2A-2G provide amino acid sequences of wild-type (FIG. 2A; SEQ ID NO:8) HLA-E heavy chains and variants (FIG. 2B-2G; SEQ ID NOs:9-14, respectively).

FIGS. 3A-3B provide an amino acid sequence of a wild-type human β2M polypeptide (FIG. 3A; SEQ ID NO: 15) and an amino acid sequence of a β2M polypeptide with an R12C substitution (FIG. 3B; SEQ ID NO: 16).

FIGS. 4A-4L provide amino acid sequences of immunoglobulin Fc polypeptides (SEQ ID NOs:17-28).

FIGS. 5A-5J provide schematic depictions of exemplary TMMPs (FIG. 5A-5G) and exemplary locations of cysteine (Cys) residues forming disulfide bonds (FIG. H-J).

FIGS. 6A-6D provide amino acid sequences of IL-2Rα (FIG. 6A; SEQ ID NO:29), IL-2Rβ (FIG. 6B; SEQ ID NO:30), and IL-2Rγ (FIG. 6C; SEQ ID NO:31), and wild-type IL-2 (FIG. 6D; SEQ ID NO:32).

FIGS. 7A-7W provide amino acid sequences of exemplary TMMPs (SEQ ID NOs:33-55).

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to polymerase chain reaction (PCR) amplification or other recombinant DNA methods. References herein to a specific residue or residue number in a known polypeptide are understood to refer to the amino acid at that position in the wild-type polypeptide. To the extent that the sequence of the wild-type polypeptide is altered, either by addition or deletion of one or more amino acids, one of ordinary skill will understand that a reference to the specific residue or residue number will be correspondingly altered so as to refer to the same specific amino acid in the altered polypeptide, which would be understood to reside at an altered position number. For example, if an MHC class I polypeptide is altered by the addition of one amino acid at the N-terminus, then a reference to position 84 or a specific residue at position 84, will be understood to indicate the amino acids that are at position 85 on the altered polypeptide. Likewise, a reference herein to substitution of a specific amino acid at a specific position, e.g., Y84, is understood to refer to a substitution of an amino acid for the amino acid at position 84 in the wild-type polypeptide. A Y84C substitution is thus understood to be a substitution of Cys residue for the Tyr residue that is present in the wild-type sequence. If, e.g., the wild-type polypeptide is altered to change the amino acid at position 84 from its wild-type amino acid to an alternate amino acid, then the substitution for the amino acid at position 84 will be understood to refer to the substitution for the alternate amino acid. If in such case the polypeptide is also altered by the addition or deletion of one or more amino acids, then the reference to the substitution will be understood to refer to the substitution for the alternate amino acid at the altered position number. A reference to a “non-naturally occurring Cys residue” in a polypeptide, e.g., an MHC class I polypeptide, means that the polypeptide comprises a Cys residue in a location where there is no Cys in the corresponding wild-type polypeptide. This can be accomplished through routine protein engineering in which a cysteine is substituted for the amino acid that occurs in the wild-type sequence.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Biol. 215:403-10. Unless otherwise stated, “sequence identity” as referred to herein is determined by BLAST (Basic Local Alignment Search Tool).

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

“T cell” includes all types of immune cells expressing CD3, including T-helper cells (CD4⁺ cells), cytotoxic T-cells (CD8⁺ cells), T-regulatory cells (Treg), and NK-T cells.

The term “immunomodulatory polypeptide” (also referred to herein as a “MOD”), as used herein, means a polypeptide that specifically binds a cognate costimulatory polypeptide on a T cell, thereby providing a signal which, in addition to the primary signal provided by, for instance, binding of a TCR/CD3 complex with a major histocompatibility complex (MHC) polypeptide loaded with peptide, mediates a T cell response, including, but not limited to, proliferation, activation, differentiation, and the like. As discussed herein, a MOD can include, but is not limited to wild-type or variants of wild-type polypeptides such as a cytokine (e.g., IL-2), CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor, and a ligand that specifically binds with B7-H3. A MOD of a TMMP can bind a cognate costimulatory polypeptide (i.e., a “co-MOD”) that is present on a target T cell.

As used herein the term “in vivo” refers to any process or procedure occurring inside of the body.

As used herein, “in vitro” refers to any process or procedure occurring outside of the body.

“Heterologous,” as used herein, means a nucleotide or polypeptide that is not found in the native nucleic acid or protein, respectively.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding,” as used herein (e.g., with reference to binding of a TMMP to a polypeptide (e.g., CD94/NKG2C, CD94/NKG2A, etc.) on a T cell), refers to a non-covalent interaction between two molecules. Non-covalent binding refers to a direct association between two molecules, due to, for example, electrostatic, hydrophobic, ionic, and/or hydrogen-bond interactions, including interactions such as salt bridges and water bridges. “Affinity” refers to the strength of non-covalent binding, increased binding affinity being correlated with a lower K_{D}. “Specific binding” generally refers to binding of a ligand to a moiety that is than its designated binding site or receptor. “Non-specific binding” generally refers to binding of a ligand to a moiety other than its designated binding site or receptor. “Covalent binding” or “covalent bond,” as used herein, refers to the formation of one or more covalent chemical binds between two different molecules.

The term “antibody” includes antibodies of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, nanobodies, bi-specific antibodies, multi-specific antibodies, and fusion proteins comprising an antigen-binding (also referred to herein as antigen binding) portion of an antibody and a non-antibody protein. The antibodies can be detectably labeled, e.g., with a radioisotope, an enzyme that generates a detectable product, a fluorescent protein, and the like. The antibodies can be further conjugated to other moieties, such as members of specific binding pairs, e.g., biotin (member of biotin-avidin specific binding pair), and the like. Also encompassed by the term are Fab′, Fv, F(ab′)₂, and or other antibody fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody can be monovalent or bivalent. An antibody can be an Ig monomer, which is a “Y-shaped” molecule that consists of four polypeptide chains: two heavy chains and two light chains connected by disulfide bonds.

The term “nanobody” (Nb), as used herein, refers to the smallest antigen binding fragment or single variable domain (V_{HH}) derived from naturally occurring heavy chain antibody and is known to the person skilled in the art. They are derived from heavy chain only antibodies, seen in camelids (Hamers-Casterman et al. (1993) Nature 363:446; Desmyter et al. (1996) Nature Structural Biol. 3:803; and Desmyter et al. (2015) Curr. Opin. Struct. Biol. 32:1). In the family of “camelids” immunoglobulins devoid of light polypeptide chains are found. “Camelids” comprise old world camelids (Camelus bactrianus and Camelus dromedarius) and new world camelids (for example, Llama paccos, Llama glama, Llama guanicoe and Llama vicugna). A single variable domain heavy chain antibody is referred to herein as a nanobody or a V_{HH} antibody.

“Antibody fragments” comprise a portion of an intact antibody, for example, the antigen binding or variable region of the intact antibody. Examples of antibody fragments include Fab, Fab′, F(ab′)₂, and Fv fragments; diabodies; linear antibodies (Zapata et al., Protein Eng. 8(10): 1057-1062 (1995)); domain antibodies (dAb; Holt et al. (2003) Trends Biotechnol. 21:484); single-chain antibody molecules; and multi-specific antibodies formed from antibody fragments. Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen combining sites and is still capable of cross-linking antigen.

“Fv” is the minimum antibody fragment that contains a complete antigen-recognition and -binding site. This region consists of a dimer of one heavy- and one light-chain variable domain in tight, non-covalent association. It is in this configuration that the three CDRS of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The “Fab” fragment also contains the constant domain of the light chain and the first constant domain (CH1) of the heavy chain. Fab fragments differ from Fab′ fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these classes can be further divided into subclasses (isotypes), e.g., IgG1, IgG2, IgG3, IgG4, IgA, and IgA2. The subclasses can be further divided into types, e.g., IgG2a and IgG2b.

“Single-chain Fv” or “sFv” or “scFv” antibody fragments comprise the V_{H} and V_{L} domains of antibody, wherein these domains are present in a single polypeptide chain. In some embodiments, the Fv polypeptide further comprises a polypeptide linker between the VH and VL domains, which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al. (1993) Proc. Natl. Acad. Sci. USA 90:6444-6448.

As used herein, the term “CDR” or “complementarity determining region” is intended to mean the non-contiguous antigen combining sites found within the variable region of both heavy and light chain polypeptides. CDRs have been described by Kabat et al (1977) J. Biol. Chem. 252:6609; Kabat et al., U.S. Dept. of Health and Human Services, “Sequences of proteins of immunological interest” (1991) (also referred to herein as Kabat 1991); by Chothia et al. (1987) J. Mol. Biol. 196:901 (also referred to herein as Chothia 1987); and MacCallum et al. (1996) J. Mol. Biol. 262:732, where the definitions include overlapping or subsets of amino acid residues when compared against each other. Nevertheless, application of either definition to refer to a CDR of an antibody or grafted antibodies or variants thereof is intended to be within the scope of the term as defined and used herein.

As used herein, the term “framework,” when used in reference to an antibody variable region, is intended to mean all amino acid residues outside the CDR regions within the variable region of an antibody. A variable region framework is generally a discontinuous amino acid sequence between about 100-120 amino acids in length but is intended to reference only those amino acids outside of the CDRs. As used herein, the term “framework region” is intended to mean each domain of the framework that is separated by the CDRs.

As used herein, the term “HLA-E heavy chain polypeptide” means collectively the domains of an HLA-E heavy chain polypeptide that are present in a TMMP. For example, an HLA-E heavy chain polypeptide can comprise α1, α2 and α3 domains.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may or may not be predisposed to acquiring the disease or symptom but has not yet been diagnosed as having it; (b) inhibiting the disease or one or more symptoms associated with the disease, e.g., arresting its development; and/or (c) relieving the disease, i.e., causing regression of the disease. The therapeutic agent may be administered before, during and/or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), etc. Unless otherwise indicated, the terms “individual,” “subject,” “host,” and “patient,” refer to a human.

Unless indicated otherwise, the term “substantially” is intended to encompass both “wholly” and “largely but not wholly”. For example, an Ig Fc that “substantially does not induce ADCC” means an Ig Fc that induces no ADCC at all or that largely does not induce ADCC.

As used herein, the term “about” used in connection with an amount indicates that the amount can vary by 10% of the stated amount. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

As used herein and in the appended claims, the singular forms “a,” “an,” and “the” include plural referents unless the context clearly dictates otherwise. Thus, for example, reference to a “TMMP” includes a plurality of such polypeptides and reference to “the immunomodulatory polypeptide” or “MOD” includes reference to one or more immunomodulatory polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the embodiments of the disclosure and does not pose a limitation on the scope of the disclosure unless otherwise claimed.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that aspects and embodiments of the present disclosure described herein include “comprising,” “consisting,” and “consisting essentially of” aspects and embodiments.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the present disclosure is not entitled to antedate such publication. Further, the dates of publication provided may be different from the actual publication dates which may need to be independently confirmed.

### DETAILED DESCRIPTION

The present disclosure provides a T-cell modulatory multimeric polypeptide (TMMP) comprising at least one heterodimer, where the heterodimer comprises first and second polypeptide chains and where the heterodimer includes an NKG2 peptide, an HLA-E heavy chain polypeptide, a beta-2 microglobulin polypeptide, and an immunoglobulin Fc polypeptide. The heterodimer can also include an immunomodulatory polypeptide and/or a cancer-targeting polypeptide. The TMMPs are useful in methods of modulating an immune response, which methods are also provided. The TMMPs are useful in methods of modulating an immune response, which methods are also provided.

### T-Cell Modulatory Multimeric Polypeptides

The present disclosure provides a TMMP comprising at least one heterodimer, where the heterodimer comprises a first polypeptide and a second polypeptide. The heterodimer includes a peptide referred to herein as an “NKG2 peptide.” The heterodimer includes class I major histocompatibility complex (MHC) polypeptides, where such polypeptides are also known as “human leukocyte antigen” (HLA) polypeptides. MHC polypeptides present in a TMMP comprise an HLA-E heavy chain polypeptide and a β2M polypeptide. The HLA-E heavy chain polypeptide and the β2M polypeptide form a peptide-MHC (pMHC) comprising the NKG2 peptide. The pMHC presents an epitope to a CD94/NKG2A and/or a CD94/NKG2C receptor. CD94/NKG2A and CD94/NKG2C receptors are present on the surface of natural killer (NK) cells. The pMHC can activate an NKG2C⁺ NK cell; thus, contacting an NK cell with a TMMP comprising the pMHC can activate an NKG2C⁺ NK cell. The heterodimer may further comprise an immunoglobulin (Ig) Fc polypeptide or other scaffold component, e.g., a scaffold component that enhances stability, manufacturability and/or serum half-life. The first polypeptide and/or the second polypeptide may include one or more independently selected peptide linkers between any two adjacent component polypeptides.

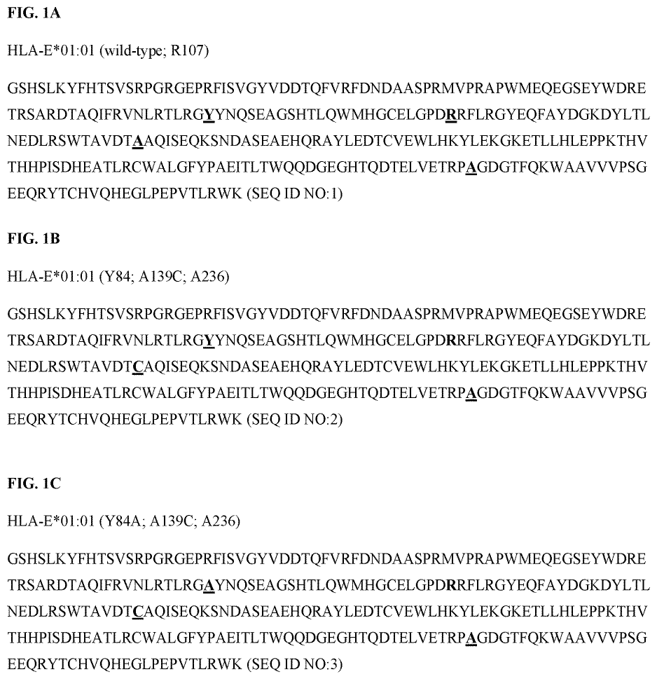

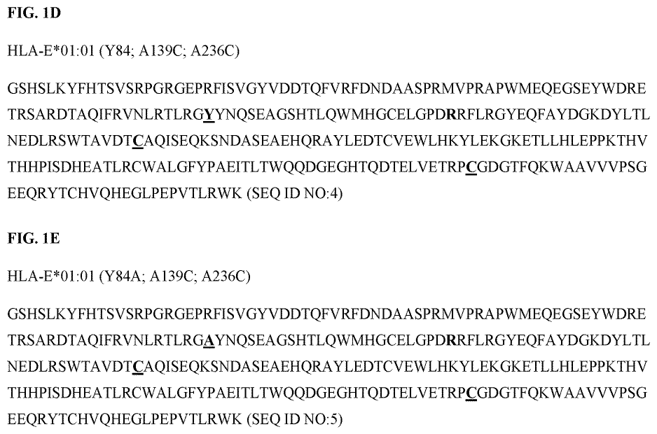

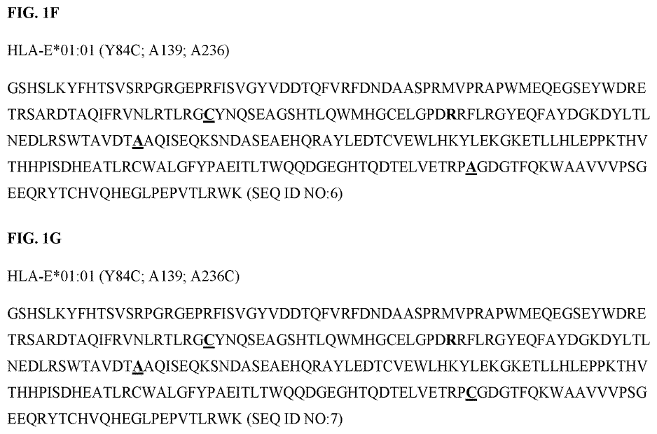

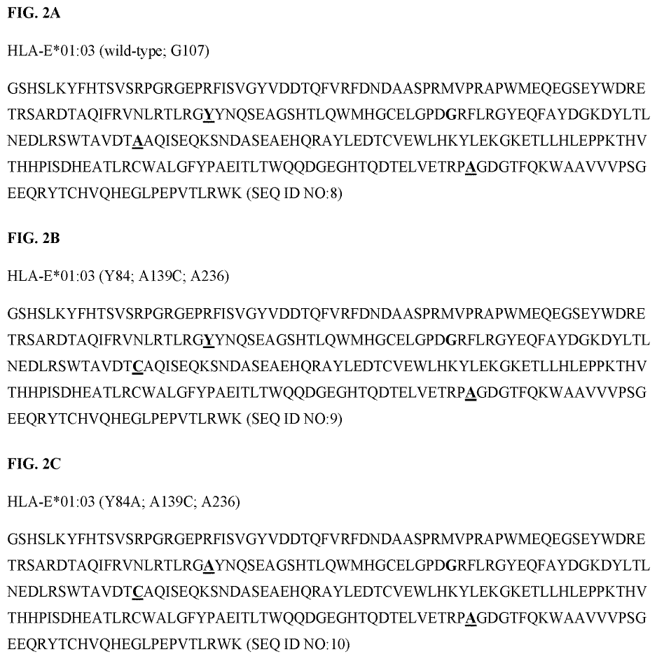

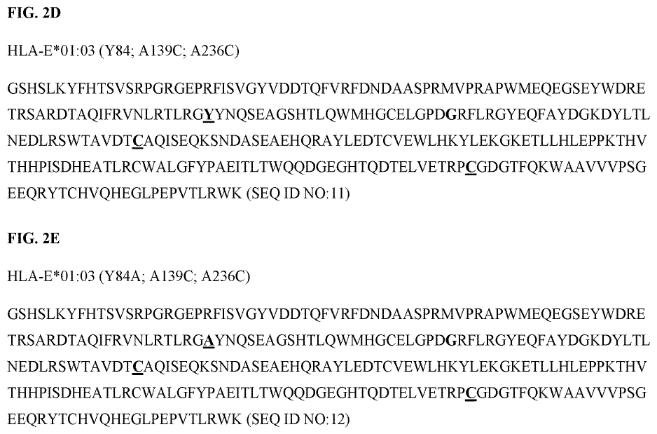

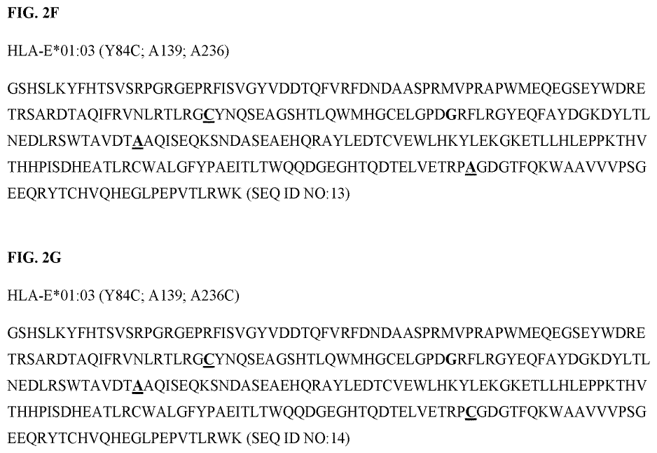

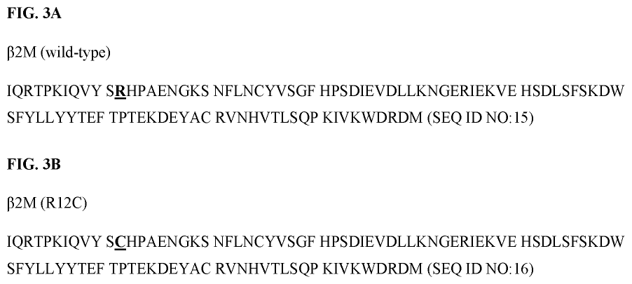

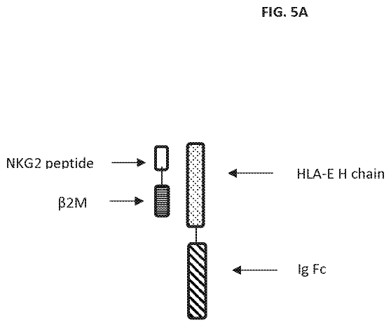

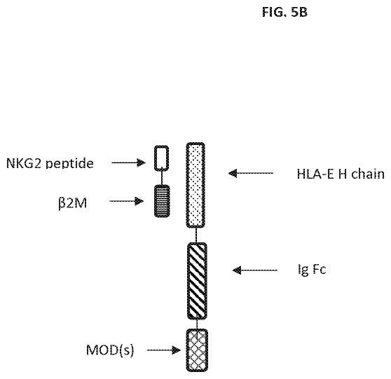

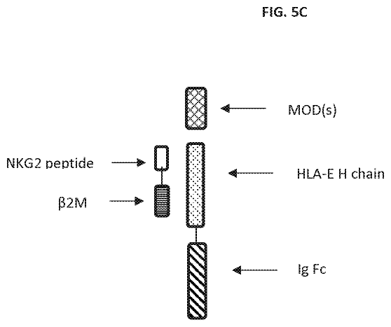

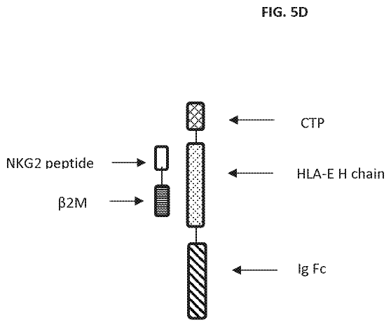

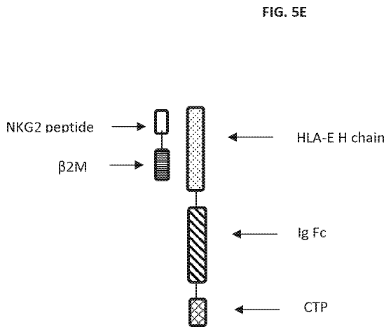

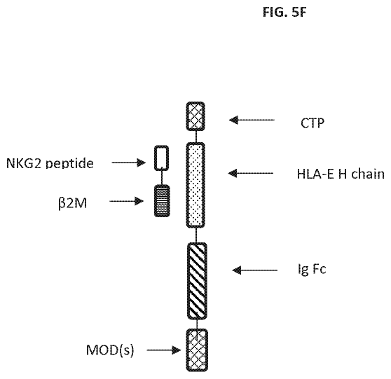

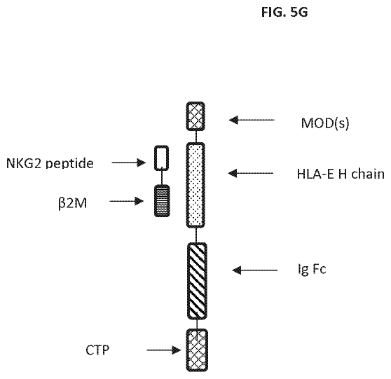

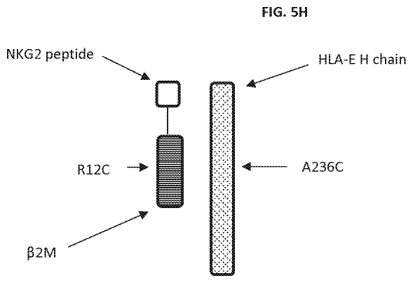

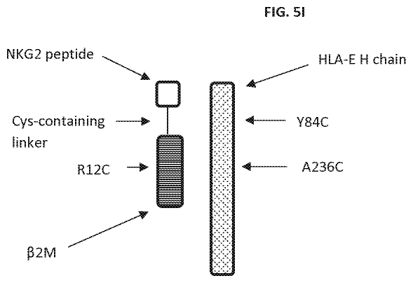

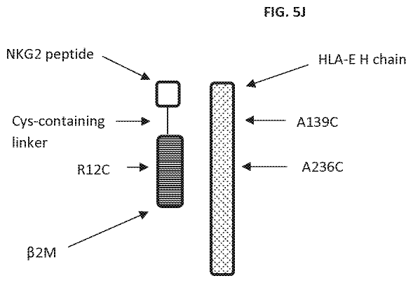

The components of a TMMP can be arranged in any of a variety of ways. For example, as depicted in FIG. 5A, in some cases, a TMMP comprises a heterodimer comprising: a) a first polypeptide comprising, in order from N-terminus to C-terminus: i) an NKG2 peptide; ii) an optional peptide linker; and iii) a β2M polypeptide; and b) a second polypeptide, comprising, in order from N-terminus to C-terminus: i) an HLA-E heavy chain polypeptide; ii) an optional peptide linker; and iii) an Ig Fc polypeptide or other scaffold polypeptide, e.g., a scaffold polypeptide that enhances stability, manufacturability and/or serum half-life. In some cases, the TMMP comprises a disulfide bond that joins a Cys residue in the β2M polypeptide to a Cys residue in the HLA-E heavy chain polypeptide. In some cases, the Cys residue in the β2M polypeptide is at amino acid 12 of the β2M polypeptide (based on the amino acid numbering set out in FIG. 3A), and the Cys residue in the HLA-E heavy chain polypeptide is at amino acid 236 of the HLA-E heavy chain polypeptide (based on the amino acid numbering set out in FIG. 1A or FIG. 2A). In some cases, the TMMP comprises: a) a first disulfide bond that joins a Cys residue in the β2M polypeptide to a Cys residue in the HLA-E heavy chain polypeptide; and b) a second disulfide bond that joins: i) a Cys residue in a peptide linker between the NKG2 peptide and the β2M polypeptide; and ii) a Cys residue in the HLA-E heavy chain polypeptide. In some cases, a) the first polypeptide comprises a peptide linker between the NKG2 peptide and the β2M polypeptide, where the peptide linker comprises a Cys; and b) the HLA-E polypeptide comprises a Cys at any one of amino acids 135-143, e.g., any one of amino acids 137-141, any one of amino acids 138-140, or amino acid 139, based on the numbering of the HLA-E polypeptide depicted in FIG. 1A or FIG. 2A, and wherein amino acid 84, based on the numbering of the HLA-E polypeptide depicted in FIG. 1A or FIG. 2A, is other than Cys, and the heterodimer comprises a disulfide bond between the Cys present in the peptide linker and the Cys at any one of amino acids 135-143 of the HLA-E polypeptide. In some cases, the HLA-E heavy chain polypeptide comprises a Cys at amino acid 138, 139, or 140 based on the numbering of the HLA-E polypeptide depicted in FIG. 1A or FIG. 2A. In some cases, the HLA-E heavy chain polypeptide comprises a Cys at amino acid 139 based on the numbering of the HLA-E polypeptide depicted in FIG. 1A or FIG. 2A.

In some cases, the TMMP comprises a disulfide bond that covalently links the first and the second polypeptide to one another. In some cases, the disulfide bond is between a Cys in the β2M polypeptide and a Cys in the HLA-E polypeptide. In some cases, the Cys in the β2M polypeptide is amino acid 12 of the β2M polypeptide, where the numbering is based on the β2M amino acid sequence depicted in FIG. 3B. In some cases, the Cys in the HLA-E is at amino acid 236, based on the numbering of the HLA-E amino acid sequence depicted in FIG. 1D. In some cases, the disulfide bond between a Cys in the β2M polypeptide and a Cys in the HLA-E polypeptide is the only disulfide bond that covalently links the first and the second polypeptide to one another.

In some cases, the TMMP comprises at least one disulfide bond, where the at least one disulfide bond is between: i) a Cys in the peptide linker between the NKG2 peptide and the β2M polypeptide; and ii) a Cys in the HLA-E heavy chain polypeptide. In some cases, the peptide linker between the NKG2 peptide and the β2M polypeptide comprises the sequence CGGGS(GGGGS)n (SEQ ID NO:126), GCGGS(GGGGS)n, (SEQ ID NO: 127) or GGCGS(GGGGS)n (SEQ ID NO: 128), wherein n is an integer from 1-10. In some cases the at least one disulfide bond is between: i) a Cys in the peptide linker between the NKG2 peptide and the β2M polypeptide; and ii) a Cys in the HLA-E heavy chain polypeptide, is the only disulfide bond that covalently links the first and the second polypeptide to one another.

In some cases, the TMMP comprises two or more disulfide bonds that covalently link the first and the second polypeptide to one another. In some cases, the TMMP comprises a first disulfide bond that covalently links the first and the second polypeptide to one another. In some cases, the first disulfide bond is between a Cys in the β2M polypeptide and a Cys in the HLA-E polypeptide. In some cases, the Cys in the β2M polypeptide is amino acid 12 of the β2M polypeptide, where the numbering is based on the β2M amino acid sequence depicted in FIG. 3B. In some cases, the Cys in the HLA-E is at amino acid 236, based on the numbering of the HLA-E amino acid sequence depicted in FIG. 1D. In some cases, a second disulfide bond is between: i) a Cys in the peptide linker between the NKG2 peptide and the β2M polypeptide; and ii) a Cys in the HLA-E heavy chain polypeptide. In some cases, the peptide linker between the NKG2 peptide and the β2M polypeptide comprises the sequence CGGGS(GGGGS)n (SEQ ID NO: 126), GCGGS(GGGGS)n (SEQ ID NO: 127), or GGCGS(GGGGS)n (SEQ ID NO: 128), wherein n is an integer from 1-10. In some cases, the second disulfide bond is formed between: i) the Cys in the peptide linker between the NKG2 peptide and the β2M polypeptide; and ii) a Cys at amino acid 84 of the HLA-E heavy chain polypeptide. In some cases, the second disulfide bond is formed between the Cys in the peptide linker and a Cys at any one of amino acids 135-143, e.g., any one of amino acids 137-141, any one of amino acids 138-140, or amino acid 139, of the HLA-E polypeptide. In some cases, the second disulfide bond is formed between: i) the Cys in the peptide linker between the NKG2 peptide and the β2M polypeptide; and ii) a Cys at amino acid 139 of the HLA-E heavy chain polypeptide. These options are depicted schematically in FIG. 5H-5J, which for simplicity show only the NKG2 peptide, the β2M polypeptide, and the HLA-E polypeptide.

### NKG2 Peptides

As noted above, a TMMP comprises an NKG2 peptide. An NKG2 peptide, when present in a pMHC comprising (i) the NKG2 peptide, (ii) an HLA-E heavy chain polypeptide, and (iii) a β2M polypeptide, presents an epitope to a CD94/NKG2A receptor and/or a CD94/NKG2C receptor.

An NKG2 peptide present in a TMMP can have a length of at least 4 amino acids, e.g., from 4-20 aa (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa or 20 aa), including a range of from 6-15 aa, 8-12 aa, 8-16 aa, 8-10 aa, 9-11 aa, 5-10 aa, 10-15 aa, and 15-20 aa in length. In some cases, the peptide epitope is 8, 9, 10, or 11 amino acids in length. In some cases, an NKG2 peptide has a length of 8 amino acids. In some cases, an NKG2 peptide has a length of 9 amino acids. In some cases, an NKG2 peptide has a length of 10 amino acids. In some cases, an NKG2 peptide has a length of 11 amino acids. In some cases, an NKG2 peptide has a length of from 8 amino acids to 12 amino acids.

Suitable NKG2 peptides include, e.g., VMAPRTLLL (SEQ ID NO:56), VMAPRALLL (SEQ ID NO:57), VMAPRTVLL (SEQ ID NO:58), and VMAPRTLVL (SEQ ID NO:59). In some cases, a peptide comprising the amino acid sequence VMAPRTLFL (SEQ ID NO:90) is specifically excluded. In some cases, a peptide comprising the amino acid sequence VMAPRTLIL (SEQ ID NO:91) is specifically excluded.

Suitable NKG2 peptides include, e.g., QMPSRSLLF (SEQ ID NO:60), TLPKRGLFL (SEQ ID NO:61), TGPWRSLWI (SEQ ID NO:62), ILTDRSLWL (SEQ ID NO:63), VNPGRSLFL (SEQ ID NO:64), VMAPRTLFL (SEQ ID NO:65), TAPARTMFL (SEQ ID NO:66), TLPERTLYL (SEQ ID NO:67), VMPPRTLLL (SEQ ID NO:68), VMPGRTLCF (SEQ ID NO:69), RMPPRSVLL (SEQ ID NO:70), NMPARTVLF (SEQ ID NO:71), and VLPHRTQFL (SEQ ID NO:72).

Suitable NKG2 peptides include, e.g., QMPSRSLLF (SEQ ID NO:73), TLPKRGLFL (SEQ ID NO:74), TGPWRSLWI (SEQ ID NO:75), ILTDRSLWL (SEQ ID NO:76), VNPGRSLFL (SEQ ID NO:77), VMAPRTLFL (SEQ ID NO:78), and TAPARTMFL (SEQ ID NO:79).

Suitable NKG2 peptides include, e.g., TLPERTLYL (SEQ ID NO:80), VMPPRTLLL (SEQ ID NO:81), VMPGRTLCF (SEQ ID NO: 82), RMPPRSVLL (SEQ ID NO:83), NMPARTVLF (SEQ ID NO:84), and VLPHRTQFL (SEQ ID NO: 85).

Suitable NKG2 peptides include, e.g., WNRLFPPLR (SEQ ID NO:86), VMGDRSVLY (SEQ ID NO:87), and VMADKSIFY (SEQ ID NO:88).

Suitable NKG2 peptides include, e.g., TGAARSFFF (SEQ ID NO:89).

In some cases, the NKG2 peptide is one that, when present in a pMHC comprising (i) the NKG2 peptide, (ii) an HLA-E heavy chain polypeptide, and (iii) a β2M polypeptide, presents an epitope preferentially to a CD94/NKG2C receptor, compared to a CD94/NKG2A receptor. A TMMP comprising such an NKG2 peptide also binds preferentially to a CD94/NKG2C receptor, compared to a CD94/NKG2A receptor. A TMMP comprising such an NKG2 peptide selectively activates an NKG2C⁺ NK cell. By “selectively activates” is meant that the TMMP activates an NKG2C⁺ NK cell to an extent that is at least 15%, at least 20%, at least 25%, at least 30%, at least 40%, or at least 50%, or more than 50%, greater than that to which an NKG2A⁺ cell is inhibited by the TMMP.

Whether NKG2C⁺ NK cells are activated or NKG2A⁺ NK cells are inhibited by a TMMP in a short term (i.e., <12 hours) can be determined by an assay described in Hammer et al. (2018) Nat. Immunol. 19:453 and Huisman et al. (2022) BioRxiv (doi: https://doi.org/10.1101/2022.08.03.502719), but using a TMMP instead of a target cell. For example, NKG2C⁻/NKG2A⁺ NK cells, NKG2C⁺/NKG2A⁺ NK cells are obtained from peripheral blood mononuclear cells (PBMCs). The purified NK cells are cultured with a TMMP at a concentration of 1 nM to 1000 nM in the presence of anti-CD107a. After the first hour of incubation, Brefeldin A and Monensin are added to inhibit secretion of proteins. After 5 additional hours of culture, the various subsets of NK cells are tested for activation or inhibition. Activation of NKG2C⁺/NKG2A⁺ NK cells, and inhibition of NKG2C⁻/NKG2A⁺ NK cells, is assessed by one or more of: i) degranulation of NK cells (as assessed by cell surface expression of CD107a); ii) production of tumor necrosis factor (TNF) by the NK cells; iii) production of interferon-gamma (IFNγ) by the NK cells; and iv) production of C-C motif chemokine ligand 3 (CCL3) by the NK cells. Activation can be expressed by the percent of NK cells that are positive for one or more of: i) CD107a; ii) TNF; iii) IFNγ; and CCL3. The percent of NK cells that are positive for the aforementioned markers can be determined using fluorochrome-labeled antibody for the surface markers/intracellular cytokines, followed by flow cytometry. In addition, the proliferation of the different subsets of NK cells are measured in terms of both the percentage among total NK cells and the cell number via flow cytometry after a 7-day culture in the presence of TMMP at a concentration of 1 nM to 1000 nM.

### HLA-E Polypeptides and β2M Polypeptides

As noted above, a TMMP comprises at least one heterodimer comprising an HLA-E heavy chain polypeptide and a β2M polypeptide.

In some cases, an HLA-E polypeptide present in a TMMP comprises an amino acid sequence having at least 85%, at least 90%, at least 95%, at least 98%, at least 99%, or 100%, amino acid sequence identity to any one of the HLA-E amino acid sequences depicted in FIG. 1A-1G (HLA-E*01:01 (wild-type; R107) and variants thereof) and FIG. 2A-2G (HLA-E*01:03 (wild-type; G107) and variants thereof).

In some cases, an HLA-E polypeptide present in a TMMP comprises an amino acid sequence having at least 85%, at least 90%, at least 95%, at least 98%, at least 99%, or 100%, amino acid sequence identity to the amino acid sequence depicted in FIG. 1B, where amino acid 84 is Tyr, amino acid 139 is Ala, and amino acid 236 is Ala. In some cases, an HLA-E polypeptide present in a TMMP comprises an amino acid sequence having at least 85%, at least 90%, at least 95%, at least 98%, at least 99%, or 100%, amino acid sequence identity to the amino acid sequence depicted in FIG. 2B, where amino acid 84 is Tyr, amino acid 139 is Ala, and amino acid 236 is Ala.

In some cases, an HLA-E polypeptide present in a TMMP comprises an amino acid sequence having at least 85%, at least 90%, at least 95%, at least 98%, at least 99%, or 100%, amino acid sequence identity to the amino acid sequence depicted in FIG. 1C, where amino acid 84 is Ala, amino acid 139 is Cys, and amino acid 236 is Ala. In some cases, an HLA-E polypeptide present in a TMMP comprises an amino acid sequence having at least 85%, at least 90%, at least 95%, at least 98%, at least 99%, or 100%, amino acid sequence identity to the amino acid sequence depicted in FIG. 2C, where amino acid 84 is Ala, amino acid 139 is Cys, and amino acid 236 is Ala.

In some cases, an HLA-E polypeptide present in a TMMP comprises an amino acid sequence having at least 85%, at least 90%, at least 95%, at least 98%, at least 99%, or 100%, amino acid sequence identity to the amino acid sequence depicted in FIG. 1D, where amino acid 84 is Tyr, amino acid 139 is Cys, and amino acid 236 is Cys. In some cases, an HLA-E polypeptide present in a TMMP comprises an amino acid sequence having at least 85%, at least 90%, at least 95%, at least 98%, at least 99%, or 100%, amino acid sequence identity to the amino acid sequence depicted in FIG. 2D, where amino acid 84 is Tyr, amino acid 139 is Cys, and amino acid 236 is Cys.

In some cases, an HLA-E polypeptide present in a TMMP comprises an amino acid sequence having at least 85%, at least 90%, at least 95%, at least 98%, at least 99%, or 100%, amino acid sequence identity to the amino acid sequence depicted in FIG. 1E, where amino acid 84 is Ala, amino acid 139 is Cys, and amino acid 236 is Cys. In some cases, an HLA-E polypeptide present in a TMMP comprises an amino acid sequence having at least 85%, at least 90%, at least 95%, at least 98%, at least 99%, or 100%, amino acid sequence identity to the amino acid sequence depicted in FIG. 2E, where amino acid 84 is Ala, amino acid 139 is Cys, and amino acid 236 is Cys.

In some cases, an HLA-E polypeptide present in a TMMP comprises an amino acid sequence having at least 85%, at least 90%, at least 95%, at least 98%, at least 99%, or 100%, amino acid sequence identity to the amino acid sequence depicted in FIG. 1F, where amino acid 84 is Cys, amino acid 139 is Ala, and amino acid 236 is Ala. In some cases, an HLA-E polypeptide present in a TMMP comprises an amino acid sequence having at least 85%, at least 90%, at least 95%, at least 98%, at least 99%, or 100%, amino acid sequence identity to the amino acid sequence depicted in FIG. 2F, where amino acid 84 is Cys, amino acid 139 is Ala, and amino acid 236 is Ala.

In some cases, an HLA-E polypeptide present in a TMMP comprises an amino acid sequence having at least 85%, at least 90%, at least 95%, at least 98%, at least 99%, or 100%, amino acid sequence identity to the amino acid sequence depicted in FIG. 1G, where amino acid 84 is Cys, amino acid 139 is Ala, and amino acid 236 is Cys. In some cases, an HLA-E polypeptide present in a TMMP comprises an amino acid sequence having at least 85%, at least 90%, at least 95%, at least 98%, at least 99%, or 100%, amino acid sequence identity to the amino acid sequence depicted in FIG. 2G, where amino acid 84 is Cys, amino acid 139 is Ala, and amino acid 236 is Cys.

In some cases, the β2M polypeptide present in a TMMP comprises an amino acid sequence having at least 85%, at least 90%, at least 95%, at least 98%, at least 99%, or 100% amino acid sequence identity to the β2M amino acid sequence depicted in FIG. 3A. In some cases, the β2M polypeptide present in a TMMP comprises an amino acid sequence having at least 85%, at least 90%, at least 95%, at least 98%, at least 99%, or 100% amino acid sequence identity to the β2M amino acid sequence depicted in FIG. 3B and comprises a Cys at amino acid 12.

### Ig Fc Polypeptides

In some cases, a TMMP comprises an Ig Fc polypeptide. An Ig Fc polypeptide is also referred to herein as an “Fc polypeptide.” The Ig Fc polypeptide of a TMMP can be a human IgG1 Fc, a human IgG2 Fc, a human IgG3 Fc, a human IgG4 Fc, etc., or a variant of a wild-type Ig Fc polypeptide. Variants include naturally-occurring variants, non-naturally-occurring variants, and combinations thereof.

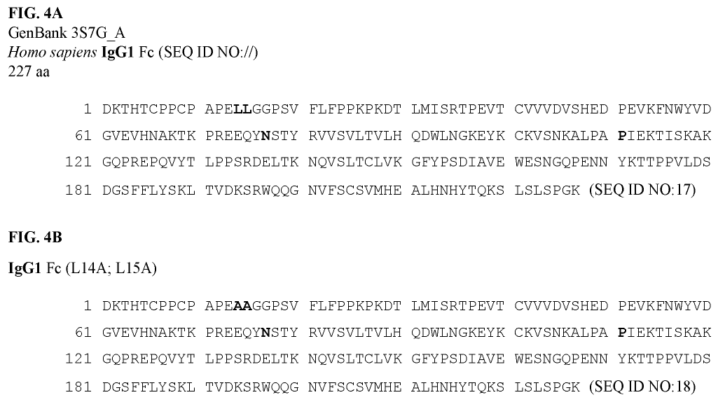

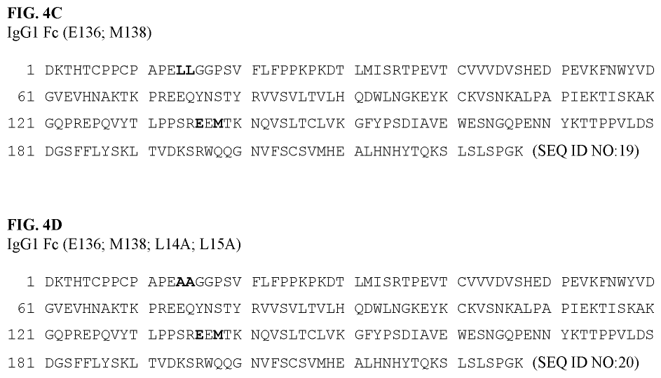

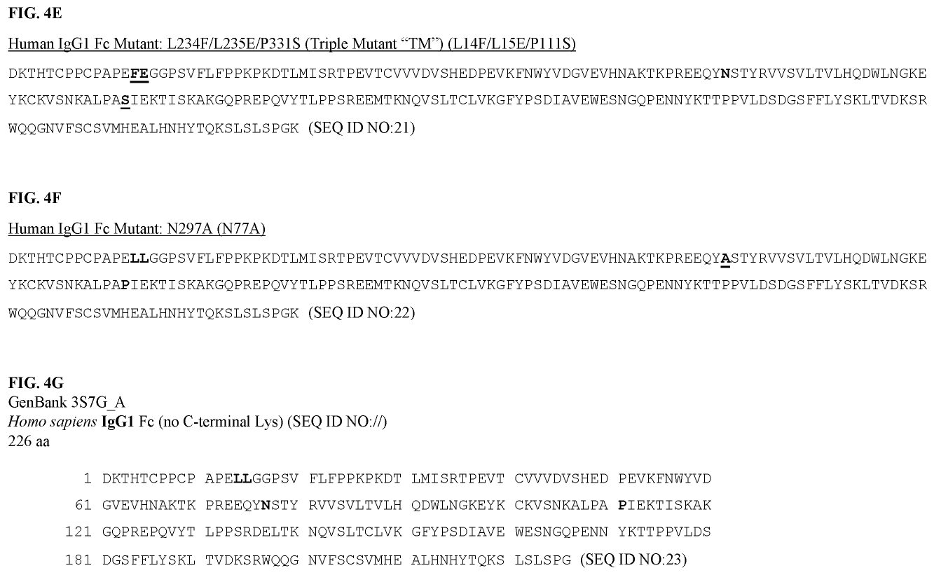

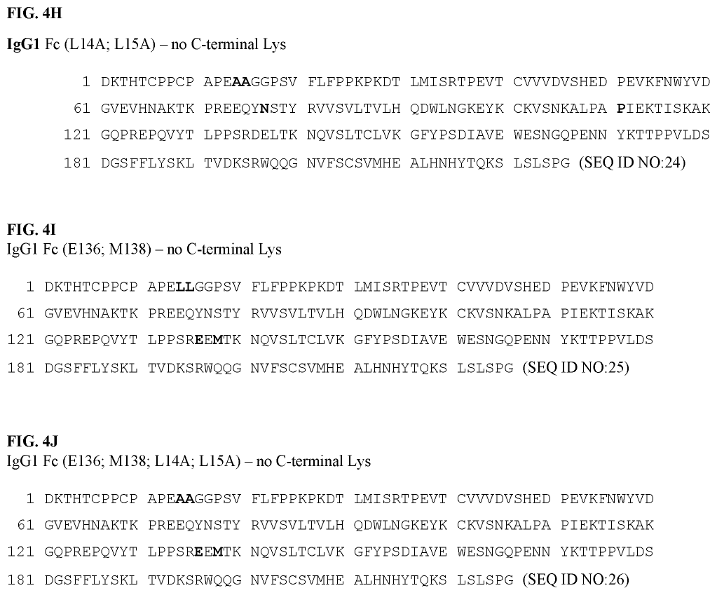

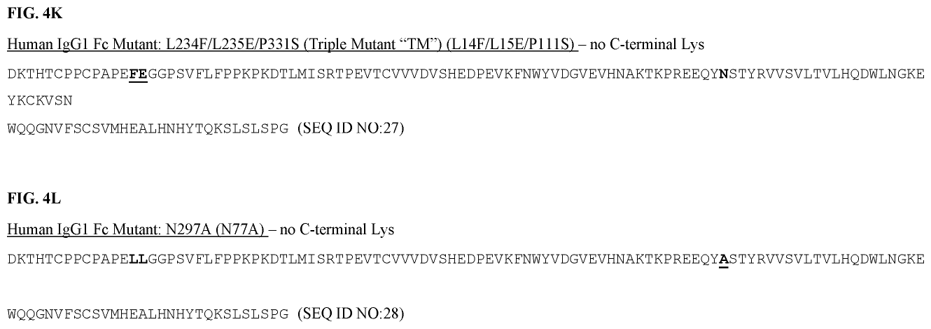

In some cases, the Fc polypeptide present in a TMMP comprises an amino acid sequence having at least about 85%, at least 90%, at least 95%, at least 98%, at least 99%, or 100%, amino acid sequence identity to the Fc amino acid sequence depicted in any one of FIG. 4A-4L.

In some cases, the Fc polypeptide present in a TMMP is an IgG1 Fc polypeptide, or a variant of an IgG1 Fc polypeptide. For example, in some cases, the Fc polypeptide present in a TMMP comprises an amino acid sequence having at least about 85%, at least 90%, at least 95%, at least 98%, at least 99%, or 100%, amino acid sequence identity to the human IgG1 Fc polypeptide depicted in FIG. 4A. As another example, in some cases, the Fc polypeptide present in a TMMP comprises an amino acid sequence having at least 85%, at least 90%, at least 95%, at least 98%, at least 99%, or 100%, amino acid sequence identity to the Fc polypeptide depicted in FIG. 4B; where the Ig Fc polypeptide comprises an Ala at position 14 and an Ala at position 15. In any of the above embodiments, the Ig Fc polypeptide can have an N77 substitution; i.e., the Ig Fc polypeptide can have an amino acid other than Asn at position 77, where in some cases, the Ig Fc polypeptide has an Ala at position 77. In some cases, an Fc polypeptide present in a TMMP comprises the amino acid sequence depicted in FIG. 4A. In some cases, an Fc polypeptide present in a TMMP comprises the amino acid sequence depicted in FIG. 4B.

In some cases, the Fc polypeptide present in a TMMP is an IgG1 Fc polypeptide, or a variant of an IgG1 Fc polypeptide, where variants include naturally-occurring variants, non-naturally-occurring variants, and combinations thereof. For example, in some cases, the Fc polypeptide present in a TMMP comprises an amino acid sequence having at least 85%, at least 90%, at least 95%, at least 98%, at least 99%, or 100%, amino acid sequence identity to the human IgG1 Fc polypeptide depicted in FIG. 4C; where the Ig Fc polypeptide comprises a Glu at position 136 and a Met at position 138. As another example, in some cases, the Fc polypeptide present in a TMMP comprises an amino acid sequence having at least 85%, at least 90%, at least 95%, at least 98%, at least 99%, or 100%, amino acid sequence identity to the human IgG1 Fc polypeptide depicted in FIG. 4D; where the Ig Fc polypeptide has Ala at positions 14 and 15; and where the Fc polypeptide comprises a Glu at position 136 and a Met at position 138. In any of the above embodiments, the Ig Fc polypeptide can have an N77 substitution; i.e., the Ig Fc polypeptide can have an amino acid other than Asn at position 77, where in some cases, the Ig Fc polypeptide has an Ala at position 77. In some cases, an Fc polypeptide present in a TMMP comprises the amino acid sequence depicted in FIG. 4C. In some cases, an Fc polypeptide present in a TMMP comprises the amino acid sequence depicted in FIG. 4D.

In some cases, the Fc polypeptide present in a TMMP comprises the amino acid sequence depicted in FIG. 4E (human IgG1 Fc comprising an L234F substitution, an L235E substitution, and a P331S substitution; where L234 corresponds to amino acid 14 of the amino acid sequence depicted in FIG. 4E; L235 corresponds to amino acid 15 of the amino acid sequence depicted in FIG. 4E; and P331 corresponds to amino acid 111 of the amino acid sequence depicted in FIG. 4E). In some cases, the Fc polypeptide present in a TMMP comprises the amino acid sequence depicted in FIG. 4F, comprising an N279A substitution (N77A of the amino acid sequence depicted in FIG. 4F).

In some cases, the Ig Fc polypeptide present in a TMMP does not include a C-terminal Lys present in a wild-type Ig Fc polypeptide. Thus, for example, in some cases, the Ig Fc polypeptide present in a TMMP comprises an amino acid sequence having at least 85%, at least 90%, at least 95%, at least 98%, at least 99%, or 100%, amino acid sequence identity to the human IgG1 Fc polypeptide depicted in any one of FIG. 4H-4L, where the Ig Fc polypeptide does not include a C-terminal Lys.

### Immunomodulatory Polypeptides (MODs)

As noted above, in some cases, a TMMP of the present disclosure may comprise one or more MODs in addition to an NGK2 peptide, an HLA-E heavy chain polypeptide, a β2M polypeptide, and an Ig Fc polypeptide. Where a TMMP comprises two (or more) MODs in tandem, in some cases, the TMMP comprises a peptide linker between the two (or more) MODs in tandem.

The components of a TMMP can be arranged in any of a variety of ways. For example, as depicted in FIG. 5B, in some cases, a TMMP comprises a heterodimer comprising: a) a first polypeptide comprising, in order from N-terminus to C-terminus: i) an NKG2 peptide; ii) an optional peptide linker; and iii) a β2M polypeptide; and b) a second polypeptide, comprising, in order from N-terminus to C-terminus: i) an HLA-E heavy chain polypeptide; ii) an optional peptide linker; iii) an Ig Fc polypeptide; iv) an optional peptide linker; and v) one or more MODs. As another example, as depicted in FIG. 5C, in some cases, a TMMP comprises a heterodimer comprising: a) a first polypeptide comprising, in order from N-terminus to C-terminus: i) an NKG2 peptide; ii) an optional peptide linker; and iii) a β2M polypeptide; and b) a second polypeptide, comprising, in order from N-terminus to C-terminus: i) one or more MODs; ii) an optional peptide linker; iii) an HLA-E heavy chain polypeptide; iv) an optional peptide linker; and v) an Ig Fc polypeptide.

In some cases, a TMMP comprises a heterodimer comprising: a) a first polypeptide comprising, in order from N-terminus to C-terminus: i) an NKG2 peptide; ii) an optional peptide linker; and iii) a β2M polypeptide; and b) a second polypeptide, comprising, in order from N-terminus to C-terminus: i) an HLA-E heavy chain polypeptide; ii) an optional peptide linker; and iii) an Ig Fc polypeptide.

In some cases, the TMMP comprises a disulfide bond that joins a Cys residue in the β2M polypeptide to a Cys residue in the HLA-E heavy chain polypeptide. In some cases, the Cys residue in the β2M polypeptide is at amino acid 12 of the β2M polypeptide (based on the amino acid numbering set out in FIG. 3A), and the Cys residue in the HLA-E heavy chain polypeptide is at amino acid 236 of the HLA-E heavy chain polypeptide (based on the amino acid numbering set out in FIG. 1A or FIG. 2A). In some cases, the TMMP comprises: a) a first disulfide bond that joins a Cys residue in the β2M polypeptide to a Cys residue in the HLA-E heavy chain polypeptide; and b) a second disulfide bond that joins: i) a Cys residue in a peptide linker between the NKG2 peptide and the β2M polypeptide; and ii) a Cys residue in the HLA-E heavy chain polypeptide. In some cases, a) the first polypeptide comprises a peptide linker between the NKG2 peptide and the β2M polypeptide, where the peptide linker comprises a Cys; and b) the HLA-E polypeptide comprises a Cys at any one of amino acids 135-143, based on the numbering of the HLA-E polypeptide depicted in FIG. 1A or FIG. 2A, and wherein amino acid 84, based on the numbering of the HLA-E polypeptide depicted in FIG. 1A or FIG. 2A, is other than Cys, and the heterodimer comprises a disulfide bond between the Cys present in the peptide linker and the Cys at any one of amino acids 135-143, e.g., any one of amino acids 137-141, any one of amino acids 138-140, or amino acid 139, of the HLA-E polypeptide. In some cases, the HLA-E heavy chain polypeptide comprises a Cys at amino acid 138, 139, or 140 based on the numbering of the HLA-E polypeptide depicted in FIG. 1A or FIG. 2A. In some cases, the HLA-E heavy chain polypeptide comprises a Cys at amino acid 139 based on the numbering of the HLA-E polypeptide depicted in FIG. 1A or FIG. 2A. These options are depicted schematically in FIG. 5H-5J, which for simplicity show only the NKG2 peptide, the β2M polypeptide, and the HLA-E polypeptide.

Where a TMMP comprises a MOD, in some cases, a MOD present in the TMMP is a wild-type (“wt”) MOD. In other cases, a MOD present in a TMMP is a variant of a wt. MOD that has reduced affinity for a co-MOD compared to the affinity of a corresponding wild-type MOD for the co-MOD. Suitable MODs that exhibit reduced affinity for a co-MOD can have from 1 amino acid (aa) to 20 aa differences from a wild-type MOD. For example, in some cases, a variant MOD present in a TMMP differs in amino acid sequence by 1 aa, 2 aa, 3 aa, 4 aa, 5 aa, 6 aa, 7 aa, 8 aa, 9 aa, or 10 aa, from a corresponding wild-type MOD. As another example, in some cases, a variant MOD present in a TMMP differs in amino acid sequence by 11 aa, 12 aa, 13 aa, 14 aa, 15 aa, 16 aa, 17 aa, 18 aa, 19 aa, or 20 aa, from a corresponding wild-type MOD.

As discussed above, a MOD may comprise a variant of a wt MOD that may exhibit reduced binding to its co-MOD, including e.g., reduced binding to one or more chains or domains of the co-MOD. For example, a variant MOD present in a TMMP may bind its co-MOD with an affinity that it at least 1000 less, at least 15% less, at least 20% less, at least 25% less, at least 30% less, at least 35% less, at least 40% less, at least 45% less, at least 5000 less, at least 5500 less, at least 60% less, at least 65% less, at least 70% less, at least 75% less, at least 80% less, at least 85% less, at least 90% less, at least 95% less, or more than 95% less, than the affinity of a corresponding wild-type MOD for the co-MOD.

Exemplary pairs of MODs and their co-MODs include, but are not limited to those set out in Table 1, below:

TABLE 1

| Immunomodulatory | |
|---|---|
| Polypeptide (MOD) | Co-MOD |
| 4-1BBL | 4-1BB |
| PD-L1 | PD-1 |
| IL-2 | IL-2 receptor |
| CD80 | CD28 |
| CD86 | CD28 |
| OX40L (CD252) | OX40 (CD134) |
| Fas ligand | Fas |
| ICOS-L | ICOS |
| ICAM | LFA-1 |
| CD30L | CD30 |
| CD40 | CD40L |
| CD83 | CD83L |
| HVEM (CD270) | CD160 |
| JAG1 (CD339) | Notch |
| JAG1 | CD46 |
| CD80 | CTLA4 |
| CD86 | CTLA4 |
| CD70 | CD27 |
| TGFβ | TGFβ receptor |

Wild-type immunomodulatory polypeptides and variants, including reduced affinity variants, such as PD-L1, CD80, CD86, 4-1BBL and IL-2 are described in the published literature, e.g., published PCT application WO2020132138A1 and WO2019/051091, the disclosures of which as they pertain to MODs and specific variant MODs of PD-L1, CD80, CD86, 4-1BBL, IL-2 are expressly incorporated herein by reference, including specifically paragraphs [00260]-[00455] of WO2020132138A1 and paragraphs [00157]-[00352] of WO2019/051091.

Of specific interest are MODs that are variants of the cytokine IL-2. Wild-type IL-2 binds to IL-2 receptor (IL-2R) on the surface of a T cell. Wild-type IL-2 has a strong affinity for IL-2R and will bind to activate most or substantially all CD8+ T cells. For this reason, synthetic forms of wild type IL-2 such as the drug Aldesleukin (trade name Proleukin®) are known to have severe side-effects when administered to humans for the treatment of cancer because the IL-2 indiscriminately activates both target and non-target T cells.

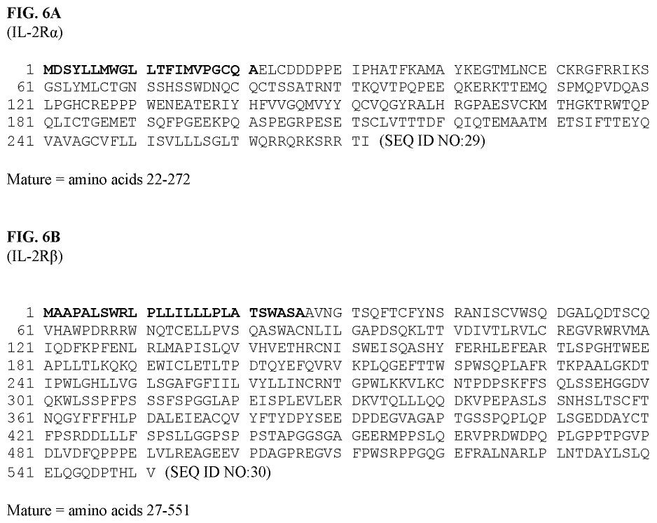

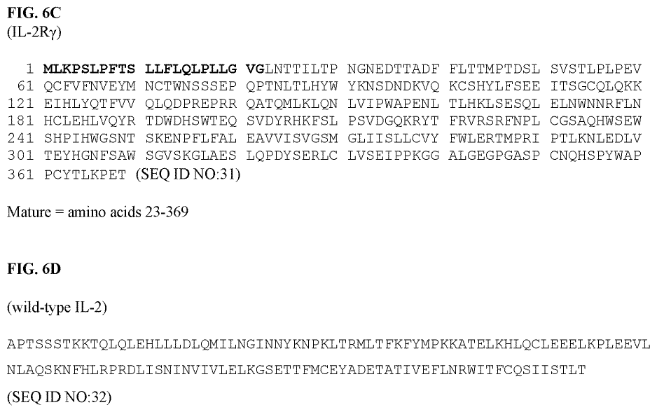

An IL-2 receptor is in some cases a heterotrimeric polypeptide comprising an alpha chain (IL-2Rα; also referred to as CD25), a beta chain (IL-2Rβ; also referred to as CD122; and a gamma chain (IL-2Rγ; also referred to as CD132). Amino acid sequences of human IL-2, human IL-2Rα, IL2Rβ, and IL-2Rγ are known. See, e.g., published PCT applications WO2020132138A1 and WO2019/051091, discussed above. For example, a wild-type IL-2 polypeptide can have the amino acid sequence depicted in FIG. 6D. Amino acid sequences of human IL-2Rα, human IL-2Rβ, and human IL-2Rγ are depicted in FIGS. 6A, 6B, and 6C, respectively, where the mature form of IL-2Rα is amino acids 22-272 of the amino acid sequence depicted in FIG. 6A, the mature form of IL-2Rβ is amino acids 27-551 of the amino acid sequence depicted in FIG. 6B, and the mature form of IL-2Rγ is amino acids 23-369 of the amino acid sequence depicted in FIG. 6C.

In some cases, an IL-2 variant MOD of this disclosure exhibits decreased binding to IL-2Rα, thereby minimizing or substantially reducing the activation of Tregs by the IL-2 variant. Alternatively, or additionally, in some cases, an IL-2 variant MOD of this disclosure exhibits decreased binding to IL-2Rβ and/or IL-2Rγ such that the IL-2 variant MOD exhibits an overall reduced affinity for IL-2R. In some cases, an IL-2 variant MOD of this disclosure exhibits both properties, i.e., it exhibits decreased or substantially no binding to IL-2Rα, and also exhibits decreased binding to IL-2Rβ and/or IL-2Rγ such that the IL-2 variant polypeptide exhibits an overall reduced affinity for IL-2R. For example, IL-2 variants having substitutions at H16 and F42 have shown decreased binding to IL-2Rα and IL-2Rβ. See, Quayle et al., Clin Cancer Res; 26(8) Apr. 15, 2020, which discloses that the binding affinity of an IL-2 polypeptide with H16A and F42A substitutions for human IL-2Rα and IL-2Rβ was decreased 110- and 3-fold, respectively, compared with wild-type IL2 binding, predominantly due to a faster off-rate for each of these interactions. TMMPs comprising such variants, including variants that exhibit decreased binding to IL-2Rα and IL-2Rβ, have shown the ability to preferentially bind to and activate IL-2 receptors on T cells that contain the target TCR that is specific for the peptide epitope on the TMMP, and are thus less likely to deliver IL-2 to non-target T cells, i.e., T cells that do not contain a TCR that specifically binds the peptide epitope on the TMMP. That is, the binding of the IL-2 variant MOD to its costimulatory polypeptide on the T cell is substantially driven by the binding of the MHC-epitope moiety to a T cell receptor rather than by the binding of the IL-2.

Suitable IL-2 variant MODs thus include a polypeptide that comprises an amino acid sequence having at least 90%, at least 95%, at least 98%, or at least 99% amino acid sequence identity to the wild-type IL-2 amino acid sequence depicted in FIG. 6D; and that have one or more amino acid differences from the wild-type IL-2 amino acid sequence depicted in FIG. 6D. In some cases, such a variant IL-2 polypeptide of this disclosure exhibits reduced binding affinity to IL-2R, compared to the binding affinity of an IL-2 polypeptide comprising the wild-type IL-2 amino acid sequence depicted in FIG. 6D. For example, in some cases, a variant IL-2 polypeptide binds IL-2R with a binding affinity that is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of an IL-2 polypeptide comprising the wild-type IL-2 amino acid sequence depicted in FIG. 6D for an IL-2R (e.g., an IL-2R comprising polypeptides comprising the amino acid sequences depicted in FIG. 6A-6C, e.g., the mature forms of the amino acid sequences depicted in FIG. 6A-6C), when assayed under the same conditions.

Some exemplary combinations of mutations that reduce binding of an IL-2 variant polypeptide to IL-2Rα and IL-2Rβ include the following from Table 2:

TABLE 2

| Mutation(s) to | Mutation(s) to | |
|---|---|---|
| decrease binding | decrease binding | |
| to IL-2Rα | to IL-2Rβ | Exemplary combinations |
| R38 with any | E15 with any | E15A with R38A, R38D or R38E |
| amino acid other | amino acid other | |
| than Arg, e.g., Ala, | than Glu | |
| Asp, Glu | | |
| R38 with any | H16, with any | H16A with R38A, R38D or R38E |
| amino acid other | amino acid other | H16T with R38A, R38D or R38E |
| than Arg, e.g., Ala, | than His, e.g., Ala, | H16E with R38A, R38D or R38E |
| Asp, Glu | Glu, Thr, or Asp, | H16D with R38A, R38D or R38E |
| R38 with any | D84, with any | D84H with R38A, R38D or R38E |
| amino acid other | amino acid other | D84K with R38A, R38D or R38E |
| than Arg, e.g., Ala, | than Asp, e.g., His, | D84R with R38A, R38D or R38E |
| Asp, Glu | Lys or Arg | |
| R38 with any | N88, with any | R38A with N88S, N88A, N88G, N88R, N88T, or N88D |
| amino acid other | amino acid other | R38D with N88S, N88A, N88G, N88R, N88T, or N88D |
| than Arg, e.g., Ala, | than Asn, e.g., Ser, | R38E with N88S, N88A, N88G, N88R, N88T, or N88D |
| Asp, Glu | Ala, Gly, Arg, Thr | |
| | or Asp | |
| R38 with any | V91 with any | R38A with V91E, V91A or V91T |
| amino acid other | amino acid other | R38D with V91E, V91A or V91T |
| than Arg, e.g., Ala, | than Val, e.g., Glu, | R38E with V91E, V91A or V91T |
| Asp, Glu | Ala or Thr | |
| R38 with any | I92 with any amino | R38A, I92A |
| amino acid other | acid other than Ile, | R38D, I92A |
| than Arg, e.g., Ala, | e.g., Ala | R38E, I92A |
| Asp, Glu | | |
| F42, with any | E15 with any | E15A, F42A |
| amino acid other | amino acid other | E15A, F42K |
| than Phe, e.g., Ala | than Glu | |
| or Lys, , as well as | | |
| Met, Pro, Ser, Thr, | | |
| Trp, Tyr, and Val | | |
| F42, with any | H16, with any | H16A, F42A; H16T, F42A; |
| amino acid other | amino acid other | H16E, F42A; H16D, F42A |
| than Phe, e.g., Ala | than His, e.g., Ala, | H16A, F42K; H16T, F42K; |
| or Lys, as well as | Glu, Thr, or Asp, | H16E, F42K; H16D, F42K |
| Met, Pro, Ser, Thr, | | |
| Trp, Tyr, and Val | | |
| F42, with any | D84, with any | F42A with D84H, D84K or D84R |
| amino acid other | amino acid other | F42K with D84H, D84K or D84R |
| than Phe, e.g., Ala | than Asp, e.g., His, | |
| or Lys, as well as | Lys or Arg | |
| Met, Pro, Ser, Thr, | | |
| Trp, Tyr, and Val | | |
| F42, with any | N88, with any | F42A with N88S, N88A, N88G, N88R, N88T, or N88D |
| amino acid other | amino acid other | F42K with N88S, N88A, N88G, N88R, N88T, or N88D |
| than Phe, e.g., Ala | than Asn, e.g., Ser, | |
| or Lys, as well as | Ala, Gly, Arg, Thr | |
| Met, Pro, Ser, Thr, | or Asp | |
| Trp, Tyr, and Val | | |
| F42, with any | V91 with any | F42A with V91E, V91A, or V91T |
| amino acid other | amino acid other | F42K with V91E, V91A, or V91T |
| than Phe, e.g., Ala | than Val, e.g., Glu, | |
| or Lys, as well as | Ala or Thr | |
| Met, Pro, Ser, Thr, | | |
| Trp, Tyr, and Val | | |
| F42, with any | I92 with any amino | F42A with I92A |
| amino acid other | acid other than Ile, | F42K with I92A |
| than Phe, e.g., Ala | e.g., Ala | |
| or Lys, as well as | | |
| Met, Pro, Ser, Thr, | | |
| Trp, Tyr, and Val | | |
| K43, with any | E15 with any | E15A, K43E |
| amino acid other | amino acid other | |
| than Lys, e.g., Glu | than Glu | |
| K43, with any | H16, with any | H16A, K43E; H16T, K43E; |
| amino acid other | amino acid other | H16E, K43E; H16D, K43E |
| than Lys, e.g., Glu | than His, e.g., Ala, | |
| | Glu, Thr, or Asp, | |
| K43, with any | D84, with any | K43E with D84H, D84K or D84R |
| amino acid other | amino acid other | |
| than Lys, e.g., Glu | than Asp, e.g., His, | |
| | Lys or Arg | |
| K43, with any | N88, with any | K43E with N88S, N88A, N88G, N88R, N88T, or N88D |
| amino acid other | amino acid other | |
| than Lys, e.g., Glu | than Asn, e.g., Ser, | |
| | Ala, Gly, Arg, Thr | |
| | or Asp | |
| K43, with any | V91 with any | K43E with V91E, V91A, or V91T |
| amino acid other | amino acid other | |
| than Lys, e.g., Glu | than Val, e.g., Glu, | |
| | Ala or Thr | |
| K43, with any | I92 with any amino | K43E, I92A |
| amino acid other | acid other than Ile, | |
| than Lys, e.g., Glu | e.g., Ala | |
| E62, with any | E15 with any | E15A, E62Q |
| amino acid other | amino acid other | |
| than Glu, e.g., Gln | than Glu | |
| E62, with any | H16, with any | H16A, E62Q; H16T, E62Q; |
| amino acid other | amino acid other | H16E, E62Q; H16D, E62Q |
| than Glu, e.g., Gln | than His, e.g., Ala, | |
| | Glu, Thr, or Asp, | |
| E62, with any | D84, with any | E62Q with D84H, D84K or D84R |
| amino acid other | amino acid other | |
| than Glu, e.g., Gln | than Asp, e.g., His, | |
| | Lys or Arg | |
| E62, with any | N88, with any | E62Q with N88S, N88A, N88G, N88R, N88T, or N88D |
| amino acid other | amino acid other | |
| than Glu, e.g., Gln | than Asn, e.g., Ser, | |
| | Ala, Gly, Arg, Thr | |
| | or Asp | |
| E62, with any | V91 with any | E62Q with V91E, V91A, or V91T |
| amino acid other | amino acid other | |
| than Glu, e.g., Gln | than Val, e.g., Glu, | |
| | Ala or Thr | |
| E62, with any | I92 with any amino | E62Q, I92A |
| amino acid other | acid other than Ile, | |
| than Glu, e.g., Gln | e.g., Ala | |
| F42, with any | E15 with any | E15A, F42A with D84H, D84K or D84R |
| amino acid other | amino acid other | E15A, F42K with D84H, D84K or D84R |
| than Phe, e.g., Ala | than Glu | |
| or Lys, as well as | D84, with any | |
| Met, Pro, Ser, Thr, | amino acid other | |
| Trp, Tyr, and Val | than Asp, e.g., His, | |
| | Lys and Arg | |
| F42, with any | E15 with any | E15A, F42A with N88S, N88A, N88G, N88R, N88T, or |
| amino acid other | amino acid other | N88D |
| than Phe, e.g., Ala | than Glu | E15A, F42K with N88S, N88A, N88G, N88R, N88T, or |
| or Lys, as well as | N88, with any | N88D |
| Met, Pro, Ser, Thr, | amino acid other | |
| Trp, Tyr, and Val | than Asn, e.g., Ser, | |
| | Ala, Gly, Arg, Thr, | |
| | and Asp | |
| F42, with any | E15 with any | E15A, F42A with V91E, V91A, or V91T |
| amino acid other | amino acid other | E15A, F42K with V91E, V91A, or V91T |
| than Phe, e.g., Ala | than Glu | |
| or Lys, as well as | V91 with any | |
| Met, Pro, Ser, Thr, | amino acid other | |
| Trp, Tyr, and Val | than Val, e.g., Glu, | |
| | Ala or Thr | |
| F42, with any | E15 with any | E15A, F42A with I92A |
| amino acid other | amino acid other | E15A, F42K with I92A |
| than Phe, e.g., Ala | than Glu | |
| or Lys, as well as | I92 with any amino | |
| Met, Pro, Ser, Thr, | acid other than Ile, | |
| Trp, Tyr, and Val | e.g., Ala | |
| F42, with any | H16, with any | H16A, F42A with D84H, D84K or D84R |
| amino acid other | amino acid other | H16A, F42K with D84H, D84K or D84R |
| than Phe, e.g., Ala | than His, e.g., Ala, | H16T, F42A with D84H, D84K or D84R |
| or Lys, as well as | Glu, Thr, or Asp | H16T, F42K with D84H, D84K or D84R |
| Met, Pro, Ser, Thr, | D84, with any | H16E, F42A with D84H, D84K or D84R |
| Trp, Tyr, and Val | amino acid other | H16E, F42K with D84H, D84K or D84R |
| | than Asp, e.g., His, | H16D, F42A with D84H, D84K or D84R |
| | Lys and Arg | H16D, F42K with D84H, D84K or D84R |
| F42, with any | H16, with any | H16A, F42A with N88S, N88A, N88G, N88R, N88T, or |
| amino acid other | amino acid other | N88D |
| than Phe, e.g., Ala | than His, e.g., Ala, | H16A, F42K with N88S, N88A, N88G, N88R, N88T, or |
| or Lys, as well as | Glu, Thr, or Asp | N88D |
| Met, Pro, Ser, Thr, | N88, with any | H16T, F42A with N88S, N88A, N88G, N88R, N88T, or |
| Trp, Tyr, and Val | amino acid other | N88D |
| | than Asn, e.g., Ser, | H16T, F42K with N88S, N88A, N88G, N88R, N88T, or |
| | Ala, Gly, Arg, Thr, | N88D |
| | and Asp | H16E, F42A with N88S, N88A, N88G, N88R, N88T, or |
| | | N88D |
| | | H16E, F42K with N88S, N88A, N88G, N88R, N88T, or |
| | | N88D |
| | | H16D, F42A with N88S, N88A, N88G, N88R, N88T, or |
| | | N88D |
| | | H16D, F42K with N88S, N88A, N88G, N88R, N88T, or |
| | | N88D |
| F42, with any | H16, with any | H16A, F42A with V91E, V91A, or V91T |
| amino acid other | amino acid other | H16A, F42K with V91E, V91A, or V91T |
| than Phe, e.g., Ala | than His, e.g., Ala, | H16T, F42A with V91E, V91A, or V91T |
| or Lys, as well as | Glu, Thr, or Asp | H16T, F42K with V91E, V91A, or V91T |
| Met, Pro, Ser, Thr, | V91 with any | H16E, F42A with V91E, V91A, or V91T |
| Trp, Tyr, and Val | amino acid other | H16E, F42K with V91E, V91A, or V91T |
| | than Val, e.g., Glu, | H16D, F42A with V91E, V91A, or V91T |
| | Ala or Thr | H16D, F42K with V91E, V91A, or V91T |
| F42, with any | H16, with any | H16A, F42A with I92A |
| amino acid other | amino acid other | H16A, F42K with I92A |
| than Phe, e.g., Ala | than His, e.g., Ala, | H16T, F42A with I92A |
| or Lys, as well as | Glu, Thr, or Asp | H16T, F42K with I92A |
| Met, Pro, Ser, Thr, | I92 with any amino | H16E, F42A with I92A |
| Trp, Tyr, and Val | acid other than Ile, | H16E, F42K with I92A |
| | e.g., Ala | H16D, F42A with I92A |
| | | H16D, F42K with I92A |

In some cases, a suitable variant IL-2 polypeptide comprises an amino acid sequence having at least 90%, at least 95%, at least 97%, at least 98%, at least 99%, or 10000 amino acid sequence identity to the amino acid sequence: APTSSSTKKT QLQLEX,LLLD LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:92), where X₁ is an amino acid other than His, and where X₂ is an amino acid other than Phe.

In some cases, a suitable variant IL-2 polypeptide comprises an amino acid sequence having at least 90%, at least 95%, at least 98%, at least 99%, or 100% amino acid sequence identity to the amino acid sequence: APTSSSTKKT QLQLEX₁LLLD LQMILNGINN YKNPKLTRML TX₂KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:93), where: i) X₁ is Ala, Arg, Asn, Asp, Cys, Gln, Glu, Gly, Ile, Leu, Lys, Met, Phe, Pro, Ser, Thr, Trp, Tyr, or Val; and ii) X₂ is Ala, Arg, Asn, Asp, Cys, Gln, Glu, Gly, His, Ile, Leu, Lys, Met, Pro, Ser, Thr, Trp, Tyr, or Val. In some cases, X₁ is Ala and X₂ is Ala. In some cases, X₁ is Thr and X₂ is Ala. In some cases, X₁ is Asp and X₂ is Ala. In some cases, X₁ is Glu and X₂ is Ala.

In some cases, a suitable variant IL-2 polypeptide comprises an amino acid sequence having at least 90%, at least 95%, at least 98%, at least 99%, or 100% amino acid sequence identity to the amino acid sequence: APTSSSTKKT QLQLEX₁LLLD LQMILNGINN YKNPKLTX₂ML TX₃KFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:94), where: i) X₁ is Ala, Arg, Asn, Asp, Cys, Gln, Glu, Gly, Ile, Leu, Lys, Met, Phe, Pro, Ser, Thr, Trp, Tyr, or Val; ii) X₂ is an amino acid other than Arg, e.g., Ala, Asp, Glu; and iii) X₃ is Ala, Arg, Asn, Asp, Cys, Gln, Glu, Gly, His, Ile, Leu, Lys, Met, Pro, Ser, Thr, Trp, Tyr, or Val. In some cases, X₁ is Ala, X₂ is Glu and X₃ is Ala. In some cases, X₁ is Thr, X₂ is Glu and X₃ is Ala. In some cases, X₁ is Asp, X₂ is Glu and X₃ is Ala. In some cases, X₁ is Glu, X₂ is Glu and X₃ is Ala.

In some cases, a suitable variant IL-2 polypeptide comprises an amino acid sequence having at least 90%, at least 95%, at least 97%, at least 98%, at least 99%, or 100% amino acid sequence identity to the amino acid sequence: APTSSSTKKT QLQLEHLLLD LQMILNGINN YKNPKLTRML TX₁KFYMPKKA TELKHLQCLE EELKPLEEVL NLAQSKNFHL RPRDLISX₂IN VIVLELKGSE TTFMCEYADE TATIVEFLNR WITFCQSIIS TLT (SEQ ID NO:95), where X₁ is an amino acid other than Phe, e.g., Ala or Lys, as well as Met, Pro, Ser, Thr, Trp, Tyr, and Val, and where X₂ is an amino acid other than Asn, e.g., Ser, Ala, Gly, Arg, Thr, and Asp. In some cases, X₁ is Ala and X₂ is Ala. In some cases, X₁ is Thr and X₂ is Ala.

In some cases, a suitable variant IL-2 polypeptide comprises an amino acid sequence having at least 90%, at least 95%, at least 97%, at least 98%, at least 99%, or 100% amino acid sequence identity to the amino acid sequence: APTSSSTKKT QLQLEHLLLD LQMILNGINN YKNPKLTX₁ML TX₂KFYMPKKA TELKHLQCLE EELKPLEEVL NLAQSKNFHL RPRDLISX₃IN VIVLELKGSE TTFMCEYADE TATIVEFLNR WITFCQSIIS TLT (SEQ ID NO:96), where X₁ is an amino acid other than Arg, e.g., Ala, Asp, Glu, X₂ is an amino acid other than Phe, e.g., Ala or Lys, as well as Met, Pro, Ser, Thr, Trp, Tyr, and Val, and where X₃ is an amino acid other than Asn, e.g., Ser, Ala, Gly, Arg, Thr, and Asp. In some cases, X₁ is Glu, X₂ is Ala and X₃ is Ala. In some cases, X₁ is Glu, X₂ is Thr and X₃ is Ala.

In some cases, a suitable variant IL-2 polypeptide comprises an amino acid sequence having at least 90%, at least 95%, at least 98%, at least 99%, or 100% amino acid sequence identity to the amino acid sequence: APTSSSTKKT QLQLEALLLD LQMILNGINN YKNPKLTRML TAKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:97), i.e., the variant IL-2 polypeptide has the amino acid sequence of wild-type IL-2 but with H16A and F42A substitutions (shown in bold). Alternatively, the foregoing sequence, but with substitutions other than Ala at H16 and/or F42 may be employed, e.g., H16T may be employed instead of H16A.

In some cases, a variant IL-2 polypeptide present in a TMMP comprises the amino acid sequence: APTSSSTKKT QLQLEALLLD LQMILNGINN YKNPKLTRML TAKFYMPKKA TELKHLQCLE EELKPLEEVL NLAQSKNFHL RPRDLISNIN VIVLELKGSE TTFMCEYADE TATIVEFLNR WITFCQSIIS TLT (SEQ ID NO:98).

In some cases, a variant IL-2 polypeptide present in a TMMP comprises the amino acid sequence: APTSSSTKKT QLQLETLLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:99).

In some cases, a TMMP comprises two copies of such a variant IL-2 polypeptide. Where a TMMP comprises two copies of a variant IL-2 polypeptide, in some cases, the two copies are in tandem. Where a TMMP comprises two copies of a variant IL-2 polypeptide, and where the two copies are in tandem, in some cases, the TMMP comprises a peptide linker between the two copies.

In some cases, a MOD present in a TMMP is a PD-L1 polypeptide. In some cases, a PD-L1 polypeptide of a TMMP comprises an amino acid sequence having at least 75%, at least 80%, at least 85%, at least 90%, at least 95%, at least 98%, at least 99%, or 100%, amino acid sequence identity to the following PD-L1 ectodomain amino acid sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO: 100).

In some cases, a MOD present in a TMMP is a 4-1BBL polypeptide. In some cases, a 4-1BBL polypeptide of a TMMP comprises an amino acid sequence having at least 75%, at least 80%, at least 85%, at least 90%, at least 95%, at least 98%, at least 99%, or 100%, amino acid sequence identity to the following 4-1BBL amino acid sequence: DPAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPA (SEQ ID NO:101).

In some cases, a MOD present in a TMMP is an ICOS-L polypeptide. In some cases, an ICOS-L polypeptide of a TMMP comprises an amino acid sequence having at least 75%, at least 80%, at least 85%, at least 90%, at least 95%, at least 98%, at least 99%, or 100%, amino acid sequence identity to the following ICOS-L amino acid sequence: QEKEVRAMVG SDVELSCACP EGSRFDLNDV YVYWQTSESK TVVTYHIPQN SSLENVDSRY RNRALMSPAG MLRGDFSLRL FNVTPQDEQK FHCLVLSQSL GFQEVLSVEV TLHVAANFSV PVVSAPHSPS QDELTFTCTS INGYPRPNVY WINKTDNSLL DQALQNDTVF LNMRGLYDVV SVLRIARTPS VNIGCCIENV LLQQNLTVGS QTGNDIGERD KITENPVSTG EKNAATWSIL (SEQ ID NO: 102).

In some cases, a MOD present in a TMMP is an OX40L polypeptide. In some cases, an OX40L polypeptide of a TMMP comprises an amino acid sequence having at least 75%, at least 80%, at least 85%, at least 90%, at least 95%, at least 98%, at least 99%, or 100%, amino acid sequence identity to the following OX40L amino acid sequence: L QVSHRYPRIQ SIKVQFTEYK KEKGFILTSQ KEDEIMKVQN NSVIINCDGF YLISLKGYFS QEVNISLHYQ KDEEPLFQLK KVRSVNSLMV ASLTYKDKVY LNVTTDNTSL DDFHVNGGEL ILIHQNPGEF CVL (SEQ ID NO: 103).

In some cases, a MOD present in a TMMP is a PD-L2 polypeptide. In some cases, a PD-L2 polypeptide of a multimeric polypeptide comprises an amino acid sequence having at least 75%, at least 80%, at least 85%, at least 90%, at least 95%, at least 98%, at least 99%, or 100%, amino acid sequence identity to amino acids 20-273 of the PD-L2 amino acid sequence: L FTVTVPKELY IIEHGSNVTL ECNFDTGSHV NLGAITASLQ KVENDTSPHR ERATLLEEQL PLGKASFHIP QVQVRDEGQY QCIIIYGVAW DYKYLTLKVK ASYRKINTHI LKVPETDEVE LTCQATGYPL AEVSWPNVSV PANTSHSRTP EGLYQVTSVL RLKPPPGRNF SCVFWNTHVR ELTLASIDLQ SQMEPRTHPT (SEQ ID NO:104).

In some cases, a MOD present in a TMMP is a CD80 polypeptide. In some cases, a CD80 polypeptide of a TMMP comprises an amino acid sequence having at least 75%, at least 80%, at least 85%, at least 90%, at least 95%, at least 98%, at least 99%, or 100%, amino acid sequence identity to following CD80 amino acid sequence: VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO: 105).

In some cases, a MOD present in a TMMP is a CD86 polypeptide. In some cases, a CD86 polypeptide of a TMMP comprises an amino acid sequence having at least 75%, at least 80%, at least 85%, at least 90%, at least 95%, at least 98%, at least 99%, or 100%, amino acid sequence identity to the following CD86 amino acid sequence:

(SEQ ID NO: 106)
APLKIQAYFNETADLPCQFANSQNQSLSELVVFWQDQENLVLNEVYLGKE
KFDSVHSKYMNRTSFDSDSWTLRLHNLQIKDKGLYQCIIHHKKPTGMIRI
HQMNSELSVLANFSQPEIVPISNITENVYINLTCSSIHGYPEPKKMSVLL
RTKNSTIEYDGIMQKSQDNVTELYDVSISLSVSFPDVTSNMTIFCILETD
KTRLLSSPFSIELEDPQPPPDHIP.

In some cases, a MOD present in a TMMP is a FasL polypeptide, e.g., the extracellular domain of a FasL polypeptide. In some cases, a FasL polypeptide of a TMMP comprises an amino acid sequence having at least 75%, at least 80%, at least 85%, at least 90%, at least 95%, at least 98%, at least 99%, or 100%, amino acid sequence identity to the following FasL extracellular domain amino acid sequence: QLFHLQKE LAELRESTSQ MHTASSLEKQ IGHPSPPPEK KELRKVAHLT GKSNSRSMPL EWEDTYGIVL LSGVKYKKGG LVINETGLYF VYSKVYFRGQ SCNNLPLSHK VYMRNSKYPQ DLVMMEGKMM SYCTTGQMWA RSSYLGAVFN LTSADHLYVN VSELSLVNFE ESQTFFGLYK L (SEQ ID NO: 107).

### TMMPs Comprising a Cancer-Targeting Polypeptide

Optionally, a TMMP may further comprise a cancer-targeting polypeptide (CTP). The CTP provides for binding to a cancer cell. By providing for binding to both an NK cell and a cancer cell, a TMMP can bring an NK cell into proximity with a cancer cell; activation of the NK cell by the TMMP can result in killing of the cancer cell.

In some cases, the first polypeptide of a TMMP that comprises a CTP comprises, in order from N-terminus to C-terminus: i) an NKG2 peptide; and ii) a β2M polypeptide. In some cases, the first polypeptide of a TMMP that comprises a CTP comprises, in order from N-terminus to C-terminus: i) an NKG2 peptide; ii) a peptide linker; and iii) a β2M polypeptide. The components of the second polypeptide of a TMMP that comprises a CTP can be arranged in any of a variety of ways; FIG. 5D and FIG. 5E provide schematic depictions of non-limiting examples. In some cases, the second polypeptide comprises, in order from N-terminus to C-terminus: i) a CTP; ii) an optional peptide linker; iii) an HLA-E heavy chain polypeptide; iv) an optional peptide linker; and v) an Ig Fc polypeptide. In some cases, the second polypeptide comprises, in order from N-terminus to C-terminus: i) an HLA-E heavy chain polypeptide; ii) an optional peptide linker; iii) an Ig Fc polypeptide; iv) an optional peptide linker; and v) a CTP. In some cases, the second polypeptide comprises, in order from N-terminus to C-terminus: i) an HLA-E heavy chain polypeptide; ii) an optional peptide linker; iii) a CTP; iv) an optional peptide linker; and v) an Ig Fc polypeptide.

A TMMP that comprises a CTP may further include one or more immunomodulatory polypeptides (“MODs”). In some cases, the first polypeptide of a TMMP that comprises a CTP and one or more MODs comprises, in order from N-terminus to C-terminus: i) an NKG2 peptide; and ii) a β2M polypeptide. In some cases, the first polypeptide comprises, in order from N-terminus to C-terminus: i) an NKG2 peptide; ii) a peptide linker; and iii) a β2M polypeptide. The components of the second polypeptide of a TMMP that comprises a CTP and one or more MODs can be arranged in any of a variety of ways; FIG. 5F and FIG. 5G provide schematic depictions of non-limiting examples. In some cases, the second polypeptide comprises, in order from N-terminus to C-terminus: i) a CTP; ii) an optional peptide linker; iii) an HLA-E heavy chain polypeptide; iv) an optional peptide linker; v) an Ig Fc polypeptide; vi) an optional peptide linker; and vii) one or more MODs. In some cases, the second polypeptide comprises, in order from N-terminus to C-terminus: i) one or more MODs; ii) an optional peptide linker; iii) an HLA-E heavy chain polypeptide; iv) an optional peptide linker; v) an Ig Fc polypeptide; vi) an optional peptide linker; and vii) a CTP. In some cases, the second polypeptide comprises, in order from N-terminus to C-terminus: i) one or more MODs; ii) an optional peptide linker; iii) an HLA-E heavy chain polypeptide; iv) an optional peptide linker; v) a CTP; vi) an optional peptide linker; and vii) an Ig Fc polypeptide.

In some cases, the first and the second polypeptide of a TMMP that comprises a CTP and one or more MODs are covalently linked to one another by one or more disulfide bonds. In some cases, the TMMP comprises a disulfide bond between a Cys in the β2M polypeptide and a Cys in the HLA-E polypeptide. In some cases, the Cys in the β2M polypeptide is amino acid 12 of the β2M polypeptide, where the numbering is based on the β2M amino acid sequence depicted in FIG. 3B. In some cases, the Cys in the HLA-E is at amino acid 236, based on the numbering of the HLA-E amino acid sequence depicted in FIG. 1D. In some cases, the disulfide bond between a Cys in the β2M polypeptide and a Cys in the HLA-E polypeptide in the only disulfide bond that links the first and second polypeptides.

In some cases, a TMMP that comprises a CTP and one or more MODs comprises a disulfide bond between: i) a Cys in the peptide linker between the NKG2 peptide and the β2M polypeptide; and ii) a Cys in the HLA-E heavy chain polypeptide. In some cases, the peptide linker between the NKG2 peptide and the β2M polypeptide comprises the sequence CGGGS(GGGGS)n (SEQ ID NO:126), GCGGS(GGGGS)n (SEQ ID NO:127), or GGCGS(GGGGS)n (SEQ ID NO: 128), wherein n is an integer from 1-10. In some cases, such disulfide bond is formed between: i) the Cys in the peptide linker between the NKG2 peptide and the β2M polypeptide; and ii) a Cys at amino acid 84 of the HLA-E heavy chain polypeptide. In some cases, such disulfide bond is formed between the Cys in the peptide linker and a Cys at any one of amino acids 135-143 of the HLA-E polypeptide. In some cases, such disulfide bond is formed between: i) the Cys in the peptide linker between the NKG2 peptide and the β2M polypeptide; and ii) a Cys at amino acid 139 of the HLA-E heavy chain polypeptide.

In some cases, the first and the second polypeptide of a TMMP that comprises a CTP and one or more MODs are covalently linked by two or more disulfide bonds. In such cases, a first disulfide bonds is located between: i) a Cys in the peptide linker between the NKG2 peptide and the β2M polypeptide; and ii) a Cys in the HLA-E heavy chain polypeptide as described above, and a second disulfide bond between a Cys in the β2M polypeptide and a Cys in the HLA-E polypeptide as described above. These options are depicted schematically in FIG. 5H-5J, which for simplicity show only the NKG2 peptide, the β2M polypeptide, and the HLA-E polypeptide.

### Cancer-Targeting Polypeptides

As noted above, a TMMP may comprise a cancer-targeting polypeptide (CTP). In some cases, the CTP is an antibody (as that term is defined above). In some cases, the CTP is an antibody that is specific for a cancer-associated antigen. In some cases, the CTP is an antibody specific for a peptide/HLA complex on the surface of a cancer cell, where the peptide can be a cancer-associated peptide (e.g., a peptide of a cancer-associated antigen).

Cancer-associated antigens that can be targeted with a CTP present in a TMP include, e.g., NY-ESO (New York Esophageal Squamous Cell Carcinoma 1), MART-1 (melanoma antigen recognized by T cells 1, also known as Melan-A), HPV (human papilloma virus) E6, BCMA (B-cell maturation antigen), CD123, CD133, CD171, CD19, CD20, CD22, CD30, CD33, CD38, CD138, CEA (carcinoembryonic antigen), EGFR (epidermal growth factor receptor), EGFRvIII (epidermal growth factor receptor variant III), EpCAM (epithelial cell adhesion molecule), EphA2 (ephrin type-A receptor 2), disialoganglioside GD2, GPC3 (glypican-3), HER2, IL13Ralpha2 (Interleukin 13 receptor subunit alpha-2), LeY (a difucosylated type 2 blood group-related antigen), MAGE-A3 (melanoma-associated antigen 3), melanoma glycoprotein, mesothelin, MUC1 (mucin 1), MUC16 (mucin-16), myelin, NKG2D (Natural Killer Group 2D) ligands, PSMA (prostate specific membrane antigen), and ROR1 (type I receptor tyrosine kinase-like orphan receptor).

Cancer-associated antigens that can be targeted with a CTP present in a TMP include, but are not limited to, 17-1A-antigen, alpha-fetoprotein (AFP), alpha-actinin-4, A3, antigen specific for A33 antibody, ART-4, B7, Ba 733, BAGE, bcl-2, bcl-6, BCMA, BrE3-antigen, CA125, CAMEL, CAP-1, carbonic anhydrase IX (CAIX), CASP-8/m, CCL19, CCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD123, CD126, CD132, CD133, CD138, CD147, CD154, CD171, CDC27, CDK-4/m, CDKN2A, CEA, CEACAM5, CEACAM6, claudin (e.g., claudin-1, claudin-10, claudin-18 (e.g., claudin-18, isoform 2)), complement factors (such as C3, C3a, C3b, C5a and C5), colon-specific antigen-p (CSAp), c-Met, CTLA-4, CXCR4, CXCR7, CXCL12, DAM, Dickkopf-related protein (DKK), ED-B fibronectin, epidermal growth factor receptor (EGFR), EGFRvIII, EGP-1 (TROP-2), EGP-2, ELF2-M, Ep-CAM, EphA2, EphA3, fibroblast activation protein (FAP), fibroblast growth factor (FGF), Flt-1, Flt-3, folate binding protein, folate receptor, G250 antigen, gangliosides (such as GC2, GD3 and GM2), GAGE, GD2, gp100, GPC3, GRO-13, HLA-DR, HM1.24, human chorionic gonadotropin (HCG) and its subunits, HER2, HER3, HMGB-1, hypoxia inducible factor (HIF-1), HIF-1a, HSP70-2M, HST-2, Ia, IFN-gamma, IFN-alpha, IFN-beta, IFN-X, IL-4R, IL-6R, IL-13R, IL13Ralpha2, IL-15R, IL-17R, IL-18R, IL-2, IL-6, IL-8, IL-12, IL-15, IL-17, IL-18, IL-23, IL-25, ILGF, ILGF-1R, insulin-like growth factor-1 (IGF-1), IGF-1R, integrin αVβ3, integrin α5β1, KC4-antigen, killer-cell immunoglobulin-like receptor (KIR), Kras, KS-1-antigen, KS1-4, LDR/FUT, Le^{gamma}macrophage migration inhibitory factor (MIF), MAGE, MAGE-3, MART-1, MART-2, mCRP, MCP-1, melanoma glycoprotein, mesothelin, MIP-1A, MIP-1B, MIF, mucins (such as MUC1, MUC2, MUC3, MUC4, MUC5ac, MUC13, MUC16, MUM-1/2 and MUM-3), NCA66, NCA95, NCA90, Nectin-4, NY-ESO-1, PAM4 antigen, pancreatic cancer mucin, PD-1, PD-L1, PD-1 receptor, placental growth factor, p53, PLAGL2, prostatic acid phosphatase, PSA, PRAME, PSMA, PIGF, RSS, RANTES, SAGE, 5100, survivin, survivin-2B, T101, TAC, TAG-72, tenascin, Thomson-Friedenreich antigens, Tn antigen, TNF-alpha, tumor necrosis antigens, TRAG-3, TRAIL receptors, vascular endothelial growth factor (VEGF), VEGF receptor (VEGFR) and WT-1.

In some cases, the cancer-associated antigen is an antigen associated with a hematological cancer. Examples of such antigens include, but are not limited to, BCMA, C5, CD19, CD20, CD22, CD25, CD30, CD33, CD38, CD40, CD45, CD52, CD56, CD66, CD74, CD79a, CD79b, CD80, CD138, CTLA-4, CXCR4, DKK, EphA3, GM2, HLA-DR beta, integrin αVβ3, IGF-R1, IL6, KIR, PD-1, PD-L1, TRAILR1, TRAILR2, transferrin receptor, and VEGF. In some cases, the cancer-associated antigen is an antigen expressed by malignant B cells, such as CD19, CD20, CD22, CD25, CD38, CD40, CD45, CD74, CD80, CTLA-4, IGF-R1, IL6, PD-1, TRAILR2, or VEGF.

In some cases, the cancer-associated antigen is an antigen associated with a solid tumor. Examples of such antigens include, but are not limited to, CAIX, cadherins, CEA, c-MET, CTLA-4, EGFR family members, EpCAM, EphA3, FAP, folate-binding protein, FR-alpha, gangliosides (such as GC2, GD3 and GM2), HER2, HER3, IGF-1R, integrin αVβ3, integrin α5β1, Le^{gamma}, Liv1, mesothelin, mucins, NaPi2b, PD-1, PD-L1, PD-1 receptor, pgA33, PSMA, RANKL, ROR1, TAG-72, tenascin, TRAILR1, TRAILR2, VEGF, VEGFR, and others listed above.

### Peptide/HLA Complexes

In some cases, the target of a CTP is a peptide/HLA (pHLA) complex on the surface of a cancer cell, where the peptide can be a cancer-associated peptide (e.g., a peptide fragment of a cancer-associated antigen). Cancer-associated peptides are known in the art. In some cases, a cancer-associated peptide is bound to an HLA complex comprising an HLA-A*0201 heavy chain and a β2M polypeptide.

In some cases, the epitope present in the pHLA on the surface of a cancer cell is bound to an HLA complex comprising an HLA heavy chain such as HLA-A*0101, A*0201, A*0301, A*1101, A*2301, A*2402, A*2407, A*3303, and/or A*3401. In some cases, the epitope present in the pHLA on the surface of a cancer cell is bound to an HLA complex comprising an HLA heavy chain such as HLA-B*0702, B*0801, B*1502, B*3802, B*4001, B*4601, and/or B*5301. In some cases, the epitope present in the pHLA on the surface of a cancer cell is bound to an HLA complex comprising an HLA heavy chain such as C*0102, C*0303, C*0304, C*0401, C*0602, C*0701, C*702, C*0801, and/or C*1502.

In some cases, the epitope is a cancer-associated epitope of any one of the following cancer-associated antigens: a MUC1 polypeptide, an LMP2 polypeptide, an epidermal growth factor receptor (EGFR) vIII polypeptide, a HER-2/neu polypeptide, a melanoma antigen family A, 3 (MAGE A3) polypeptide, a p53 polypeptide, a mutant p53 polypeptide, an NY-ESO-1 polypeptide, a folate hydrolase (prostate-specific membrane antigen; PSMA) polypeptide, a carcinoembryonic antigen (CEA) polypeptide, a claudin polypeptide (e.g., claudin-1, claudin-10, claudin-18 (e.g., claudin-18, isoform 2)), a Nectin-4 polypeptide, a melanoma antigen recognized by T-cells (melanA/MART1) polypeptide, a Ras polypeptide, a gp100 polypeptide, a proteinase3 (PRI) polypeptide, a bcr-abl polypeptide, a tyrosinase polypeptide, a survivin polypeptide, a prostate specific antigen (PSA) polypeptide, an hTERT polypeptide, a sarcoma translocation breakpoints polypeptide, a synovial sarcoma X (SSX) breakpoint polypeptide, an EphA2 polypeptide, an acid phosphatase, prostate (PAP) polypeptide, a melanoma inhibitor of apoptosis (ML-IAP) polypeptide, an epithelial cell adhesion molecule (EpCAM) polypeptide, an ERG (TMPRSS2 ETS fusion) polypeptide, a NA17 polypeptide, a paired-box-3 (PAX3) polypeptide, an anaplastic lymphoma kinase (ALK) polypeptide, an androgen receptor polypeptide, a cyclin B1 polypeptide, an N-myc proto-oncogene (MYCN) polypeptide, a Ras homolog gene family member C (RhoC) polypeptide, a tyrosinase-related protein-2 (TRP-2) polypeptide, a mesothelin polypeptide, a prostate stem cell antigen (PSCA) polypeptide, a melanoma associated antigen-1 (MAGE A1) polypeptide, a cytochrome P450 1B1 (CYP1B1) polypeptide, a placenta-specific protein 1 (PLAC1) polypeptide, a BORIS polypeptide (also known as CCCTC-binding factor or CTCF), an ETV6-AML polypeptide, a breast cancer antigen NY-BR-1 polypeptide (also referred to as ankyrin repeat domain-containing protein 30A), a regulator of G-protein signaling (RGS5) polypeptide, a squamous cell carcinoma antigen recognized by T-cells (SART3) polypeptide, a carbonic anhydrase IX polypeptide, a paired box-5 (PAX5) polypeptide, an OY-TES1 (testis antigen; also known as acrosin binding protein) polypeptide, a sperm protein 17 polypeptide, a lymphocyte cell-specific protein-tyrosine kinase (LCK) polypeptide, a high molecular weight melanoma associated antigen (HMW-MAA), an A-kinase anchoring protein-4 (AKAP-4), a synovial sarcoma X breakpoint 2 (SSX2) polypeptide, an X antigen family member 1 (XAGE1) polypeptide, a B7 homolog 3 (B7H3; also known as CD276) polypeptide, a legumain polypeptide (LGMN1; also known as asparaginyl endopeptidase), a tyrosine kinase with Ig and EGF homology domains-2 (Tie-2; also known as angiopoietin-1 receptor) polypeptide, a P antigen family member 4 (PAGE4) polypeptide, a vascular endothelial growth factor receptor 2 (VEGF2) polypeptide, a MAD-CT-1 polypeptide, a fibroblast activation protein (FAP) polypeptide, a platelet derived growth factor receptor beta (PDGFβ) polypeptide, a MAD-CT-2 polypeptide, a Fos-related antigen-1 (FOSL) polypeptide; a human papilloma virus (HPV) antigen; an alpha-feto protein (AFP) antigen; and a Wilms tumor-1 (WTi) antigen. Examples of polypeptides that comprise cancer-associated peptides of mesothelin, HER-2, BCMA, WTi, HPV and claudin-18 (isoform2) are antibodies for HER2, mesothelin, PSMA, TROP-2, BCMA, MUC1, MUC16, Claudin18.2 and TROP-2 are provided in WO 2023/097188 (Cue Biopharma, Inc.), published Jun. 1, 2023, at paragraphs [00252]-[00264], the contents of which are expressly incorporated herein by reference.

For example, in some cases, a CTP present in a TMP binds to: a) a WT-1 peptide bound to an HLA complex comprising an HLA heavy chain (e.g., an HLA-A*0201 heavy chain or an HLA-A*2402 heavy chain) and a β2M polypeptide; b) an HPV peptide bound to an HLA complex comprising a class I HLA heavy chain and a β2M polypeptide; c) a mesothelin peptide bound to an HLA complex comprising a class I HLA heavy chain and a β2M polypeptide; d) a Her2 peptide bound to an HLA complex comprising a class I HLA heavy chain and a β2M polypeptide; or e) a BCMA peptide bound to an HLA complex comprising a class I HLA heavy chain and a β2M polypeptide.

Non-limiting examples of cancer-associated antigen-targeted antibodies that can be included in a TMMP include, but are not limited to, abituzumab (anti-CD51), LL1 (anti-CD74), LL2 or RFB4 (anti-CD22), veltuzumab (hA20, anti-CD20), binutuzumab (GA101, anti-CD20), daratumumab (anti-CD38), lambrolizumab (anti-PD-1 receptor), nivolumab (anti-PD-1 receptor), ipilimumab (anti-CTLA-4), RS7 (anti-TROP-2), PAM4 or KC4 (both anti-mucin), MN-14 (anti-CEA), MN-15 or MN-3 (anti-CEACAM6), Mu-9 (anti-colon-specific antigen-p), Immu31 (anti-alpha-fetoprotein), RI (anti-IGF-1R), A19 (anti-CD19), TAG-72 (e.g., CC49), J591 or HuJ591 (anti-PSMA), AB-PGI-XG1-026 (anti-PSMA dimer), D2/B (anti-PSMA), G250 (anti-carbonic anhydrase IX), L243 (anti-HLA-DR) alemtuzumab (anti-CD52), oportuzumab (anti-EpCAM), bevacizumab (anti-VEGF), cetuximab (anti-EGFR), gemtuzumab (anti-CD33), ibritumomab tiuxetan (anti-CD20); panitumumab (anti-EGFR); tositumomab (anti-CD20); PAM4 (also known as clivatuzumab; anti-mucin), trastuzumab (anti-HER2), pertuzumab (anti-HER2), polatuzumab (anti-CD79b), and anetumab (anti-mesothelin).

In some cases, the CTP is a single-chain antibody. In some cases, the CTP is a scFv. In some cases, the CTP is a nanobody (also referred to as a single domain antibody (sdAb)). In some cases, the CTP is a heavy chain nanobody. In some cases, the CTP is a light chain nanobody. In some cases, the CTP is a diabody.

VH and VL amino acid sequences of various cancer antigen-binding antibodies are known in the art, as are the light chain and heavy chain CDRs of such antibodies. See, e.g., Ling et al. (2018) Frontiers Immunol. 9:469; WO 2005/012493; US 2019/0119375; US 2013/0066055. Examples of antibodies to HER2, mesothelin, PSMA, TROP-2, BCMA, MUC1, MUC16, and Claudin18.2 are provided in WO 2023/097188 (Cue Biopharma, Inc.), published Jun. 1, 2023 at paragraphs [00269]-[00364], the contents of which are expressly incorporated herein by reference.

### Peptide Linkers

As noted above, a TMMP can comprise one or more independently selected peptide linkers between any two components of a TMMP. For example, where a TMMP comprises an NKG2 peptide, a β2M polypeptide, an HLA-E polypeptide, and an Ig Fc polypeptide, the TMMP can comprise a peptide linker between one or both of: i) the NKG2 peptide and the β2M polypeptide; and ii) the HLA-E polypeptide and the Ig Fc polypeptide. Where a TMMP comprises an NKG2 peptide, a β2M polypeptide, an HLA-E polypeptide, one or more MODs, and an Ig Fc polypeptide, the TMMP can comprise a peptide linker between one or more of: i) the NKG2 peptide and the β2M polypeptide; ii) the HLA-E polypeptide and the Ig Fc polypeptide; iii) the HLA-E polypeptide and a MOD; iv) the Ig Fc polypeptide and a MOD; and v) 2 MODs that are in tandem. As discussed below, a peptide linker may be a flexible peptide linker, including a short flexible peptide linker, or a rigid peptide linker. As discussed below, a peptide linker may be a rigid peptide linker.

Suitable linkers (also referred to as “spacers”) can be readily selected and can be of any of a number of suitable lengths, such as from 1 amino acid to 25 amino acids, from 3 amino acids to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids. A suitable linker can be 1, 2, 3, 4, 5, 6, 7, 8, 9, 10, 11, 12, 13, 14, 15, 16, 17, 18, 19, 20, 21, 22, 23, 24, or 25 amino acids in length. In some cases, a linker has a length of from 25 amino acids to 50 amino acids, e.g., from 25 to 30, from 30 to 35, from 35 to 40, from 40 to 45, or from 45 to 50 amino acids in length.

### Cysteine-Containing Linkers

As discussed above, in some cases, a TMMP comprises a peptide linker between the NKG2 peptide and the β2M polypeptide, where the peptide linker comprises a Cys and may be referred to as a “Cys-containing peptide linker.” In some cases, a Cys-containing peptide linker comprises the amino acid sequence CGGGS(GGGGS)n (SEQ ID NO: 126), GCGGS(GGGGS)n (SEQ ID NO: 127), or GGCGS(GGGGS)n (SEQ ID NO: 128), where n is an integer from 1-10 (i.e., where n is 1, 2, 3, 4, 5, 6, 7, 8, 9, or 10). In some cases, a Cys-containing peptide linker comprises the amino acid sequence CGGGS(GGGGS)n (SEQ ID NO: 126), where n is an integer from 1-10 (i.e., where n is 1, 2, 3, 4, 5, 6, 7, 8, 9, or 10); such a peptide linker is referred to as a “GIC” linker. In some cases, a Cys-containing peptide linker comprises the amino acid sequence CGGGS(GGGGS)n (SEQ ID NO:126), where n is 1. In some cases, a Cys-containing peptide linker comprises the amino acid sequence CGGGS(GGGGS)n (SEQ ID NO: 126), where n is 2. In some cases, a Cys-containing peptide linker comprises the amino acid sequence CGGGS(GGGGS)n (SEQ ID NO: 126), where n is 3. In some cases, a Cys-containing peptide linker comprises the amino acid sequence GCGGS(GGGGS)n (SEQ ID NO:127), where n is an integer from 1-10 (i.e., where n is 1, 2, 3, 4, 5, 6, 7, 8, 9, or 10); such a peptide linker is referred to as a “G2C” linker. In some cases, a Cys-containing peptide linker comprises the amino acid sequence GCGGS(GGGGS)n (SEQ ID NO: 127), where n is 1. In some cases, a Cys-containing peptide linker comprises the amino acid sequence GCGGS(GGGGS)n (SEQ ID NO:127), where n is 2. In some cases, a Cys-containing peptide linker comprises the amino acid sequence GCGGS(GGGGS)n (SEQ ID NO:127), where n is 3. In some cases, a Cys-containing peptide linker comprises the amino acid sequence GGCGS(GGGGS)n (SEQ ID NO: 128), where n is an integer from 1-10 (i.e., where n is 1, 2, 3, 4, 5, 6, 7, 8, 9, or 10); such a linker is referred to as a “G3C” linker. In some cases, a Cys-containing peptide linker comprises the amino acid sequence GGCGS(GGGGS)n (SEQ ID NO: 128), where n is 1. In some cases, a Cys-containing peptide linker comprises the amino acid sequence GGCGS(GGGGS)n (SEQ ID NO: 128), where n is 2. In some cases, a Cys-containing peptide linker comprises the amino acid sequence GGCGS(GGGGS)n (SEQ ID NO: 128), where n is 3.

### Flexible Peptide Linkers

Exemplary flexible peptide linkers include glycine polymers (G)ₙ, glycine-serine polymers (including, for example, (GS)ₙ, (GSGGS)ₙ (SEQ ID NO: 108), (GGGGS)ₙ (SEQ ID NO: 109), and (GGGS)ₙ (SEQ ID NO:110), where n is an integer of at least one and can be an integer from 1 to 10), glycine-alanine polymers, alanine-serine polymers, and other flexible peptide linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary flexible peptide linkers can comprise amino acid sequences including, but not limited to, GGSG (SEQ ID NO:111), GGSGG (SEQ ID NO: 112), GSGSG (SEQ ID NO: 113), GSGGG (SEQ ID NO: 114), GGGSG (SEQ ID NO: 115), GSSSG (SEQ ID NO: 116), GGGGS (SEQ ID NO: 149), and the like.

Exemplary flexible peptide linkers include, e.g., (GGGGS)ₙ (SEQ ID NO: 109); also referred to as a “G4S” linker), where n is 1, 2, 3, 4, 5, 6, 7, 8, 9, or 10. In some cases, a linker comprises the amino acid sequence (GGGGS)n (SEQ ID NO: 109), where n is 1, 2, 3, 4, 5, 6, 7, 8, 9 or 10. In some cases, a linker comprises the amino acid sequence (GGGGS)n (SEQ ID NO:109), where n is 2. In some cases, a linker comprises the amino acid sequence (GGGGS)n (SEQ ID NO:109), where n is 3. In some cases, a linker comprises the amino acid sequence (GGGGS)n (SEQ ID NO: 109), where n is 4. In some cases, a linker comprises the amino acid sequence (GGGGS)n (SEQ ID NO: 109), where n is 7. In some cases, a linker comprises the amino acid sequence AAAGG (SEQ ID NO: 117). Also suitable is a linker having the amino acid sequence AAAGG (SEQ ID NO: 117). In some embodiments of a TMMP of this disclosure, the β2M polypeptide can be connected to the MHC heavy chain polypeptide by a (GGGGS)n (SEQ ID NO: 109) linker, where n is 1, 2, 3, 4, 5, 6, 7, 8, 9, or 10, e.g., where n=2, n=3, n=4, or n=7.

As used in this disclosure, a “short flexible peptide linker” means a flexible peptide linker that comprises fewer than 15 amino acids, i.e., from 2-14 amino acids. For example, a short flexible peptide linker can comprise from 2-4, 2-5, or 3-6 amino acids (e.g., a GGS linker), or from 4-8, 5-10 or from 10-14 amino acids. Within this range includes flexible peptide linkers comprising 2, 3, 4, 5, 6, 7, 8, 9, 10, 11, 12, 13 or 14 amino acids. Such short flexible peptide linkers can be used to reduce or substantially eliminate unfavorable interactions between protein domains within a TMMP.

### Rigid Peptide Linkers

In some cases, a peptide linker is a rigid peptide linker. As used herein, the term “rigid peptide linker” refers to a linker comprising a contiguous stretch of two or more amino acids that effectively separates protein domains by maintaining a substantially fixed distance/spatial separation (or a minimum fixed distance/spatial separation) between the domains, thereby reducing or substantially eliminating unfavorable interactions between such domains. Rigid peptide linkers are known in the art and generally adopt a relatively well-defined conformation when in solution. Rigid peptide linkers include those which have a particular secondary and/or tertiary structure in solution; and are typically of a length sufficient to confer secondary or tertiary structure to the linker. Rigid peptide linkers include peptide linkers rich in proline, and peptide linkers having an inflexible helical structure, such as an α-helical structure. Rigid peptide linkers are described in, for example, Chen et al. (2013) Adv. Drug Deliv. Rev. 65:1357; and Klein et al. (2014) Protein Engineering, Design & Selection 27:325.

Examples of rigid peptide linkers include, e.g., (EAAAK)n (SEQ ID NO: 118), A(EAAAK)n (SEQ ID NO: 119), A(EAAAK)nA (SEQ ID NO:120), A(EAAAK)nALEA(EAAAK)nA (SEQ ID NO:121), (Lys-Pro)n (SEQ ID NO:122), (Glu-Pro)n (SEQ ID NO: 123), (Thr-Pro-Arg)n (SEQ ID NO:124), and (Ala-Pro)n (SEQ ID NO: 125) where n is an integer from 1 to 20 (e.g., n is 1, 2, 3, 4, 5, 6, 7, 8, 9, 10, 11, 12, 13, 14, 15, 16, 17, 18, 19, or 20). Non-limiting examples of suitable rigid peptide linkers comprising EAAAK (SEQ ID NO: 129) include EAAAK (SEQ ID NO: 129), (EAAAK)₂ (SEQ ID NO: 130), (EAAAK)₃ (SEQ ID NO: 131), A(EAAAK)₄ALEA(EAAAK)₄A (SEQ ID NO: 132), and AEAAAKEAAAKA (SEQ ID NO: 133). Non-limiting examples of suitable rigid peptide linkers comprising (AP)n include PAPAP (SEQ ID NO: 134); also referred to herein as “(AP)2”); APAPAPAP (SEQ ID NO: 135); also referred to herein as “(AP)4”); APAPAPAPAPAP (SEQ ID NO: 136); also referred to herein as “(AP)6”); APAPAPAPAPAPAPAP (SEQ ID NO: 137); also referred to herein as “(AP)8”); and APAPAPAPAPAPAPAPAPAP (SEQ ID NO: 138); also referred to herein as “(AP)10”). Non-limiting examples of suitable rigid peptide linkers comprising (KP)n include KPKP (SEQ ID NO: 139); also referred to herein as “(KP)2”); KPKPKPKP (SEQ ID NO: 140); also referred to herein as “(KP)4”); KPKPKPKPKPKP (SEQ ID NO: 141); also referred to herein as “(KP)6”); KPKPKPKPKPKPKPKP (SEQ ID NO: 142); also referred to herein as “(KP)8”); and KPKPKPKPKPKPKPKPKPKP (SEQ ID NO: 143); also referred to herein as “(KP)10”). Non-limiting examples of suitable rigid peptide linkers comprising (EP)n include EPEP (SEQ ID NO: 144); also referred to herein as “(EP)2”); EPEPEPEP (SEQ ID NO: 145); also referred to herein as “(EP)4”); EPEPEPEPEPEP (SEQ ID NO: 146); also referred to herein as “(EP)6”); EPEPEPEPEPEPEPEP (SEQ ID NO: 147); also referred to herein as “(EP)8”); and EPEPEPEPEPEPEPEPEPEP (SEQ ID NO: 148); also referred to herein as “(EP)10”).

### Disulfide Bonds

The first and the second polypeptide of a heterodimer of a TMMP can be covalently linked to one another by one or more disulfide bonds.

In some cases, the first and the second polypeptides are covalently linked to one another by a single disulfide bond. In some cases, the single disulfide bond is between a Cys in the β2M polypeptide and a Cys in the HLA-E polypeptide. In some cases, the Cys in the β2M polypeptide is amino acid 12 of the β2M polypeptide, where the numbering is based on the β2M amino acid sequence depicted in FIG. 3B. In some cases, the Cys in the HLA-E is at amino acid 236, based on the numbering of the HLA-E amino acid sequence depicted in FIG. 1D. In some cases, the first and the second polypeptide are covalently linked to one another by 2 disulfide bonds. In some cases, the second disulfide bond is between: i) a Cys in the peptide linker between the NKG2 peptide and the β2M polypeptide; and ii) a Cys in the HLA-E heavy chain polypeptide. In some cases, the peptide linker between the NKG2 peptide and the β2M polypeptide comprises the sequence CGGGS(GGGGS)ₙ (SEQ ID NO:126), GCGGS(GGGGS)ₙ (SEQ ID NO:127), or GGCGS(GGGGS)ₙ (SEQ ID NO: 128), wherein n is an integer from 1-10. In some cases, the second disulfide bond is formed between: i) the Cys in the peptide linker between the NKG2 peptide and the β2M polypeptide; and ii) a Cys at amino acid 84 of the HLA-E heavy chain polypeptide. In some cases, the second disulfide bond is formed between the Cys in the peptide linker and a Cys at any one of amino acids 135-143 of the HLA-E polypeptide. In some cases, the second disulfide bond is formed between: i) the Cys in the peptide linker between the NKG2 peptide and the β2M polypeptide; and ii) a Cys at amino acid 139 of the HLA-E heavy chain polypeptide. These options are depicted schematically in FIG. 5H-5J, which for simplicity show only the NKG2 peptide, the β2M polypeptide, and the HLA-E polypeptide.

### Exemplary TMMPs

The following are non-limiting examples of TMMPs.

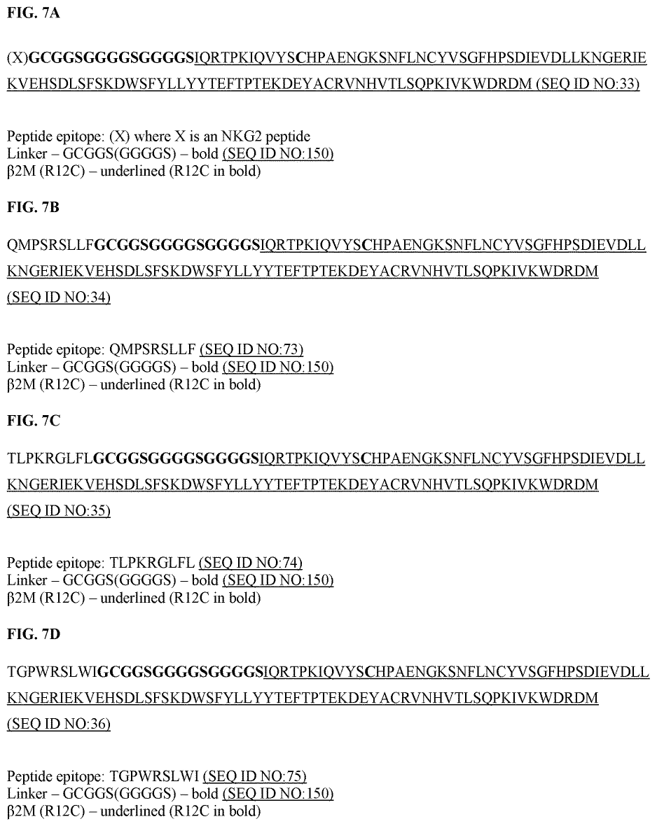

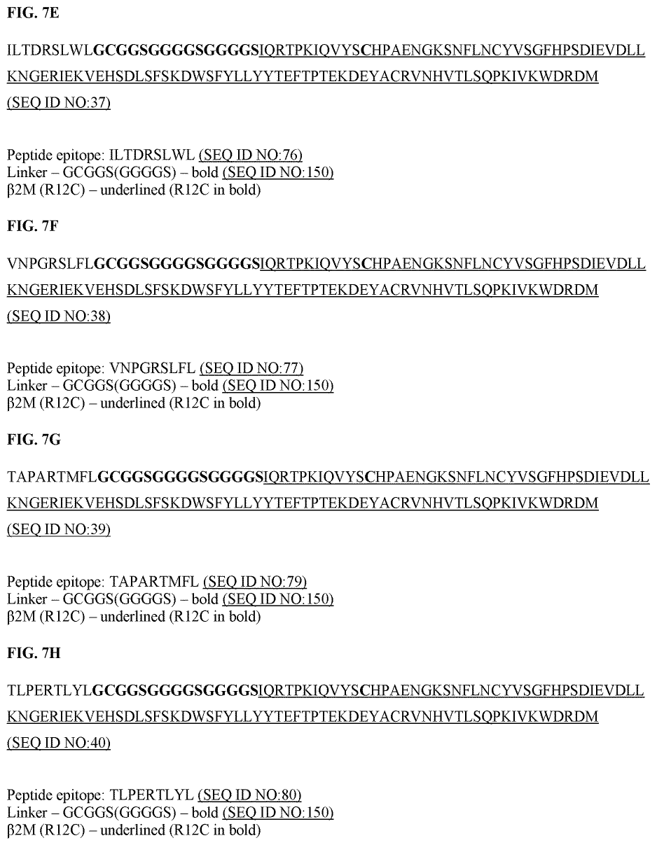

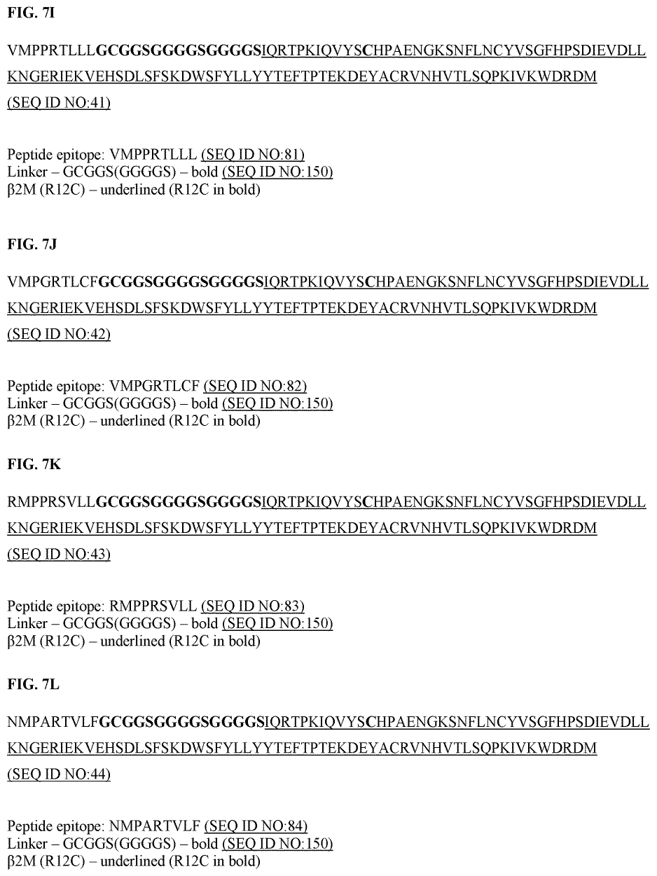

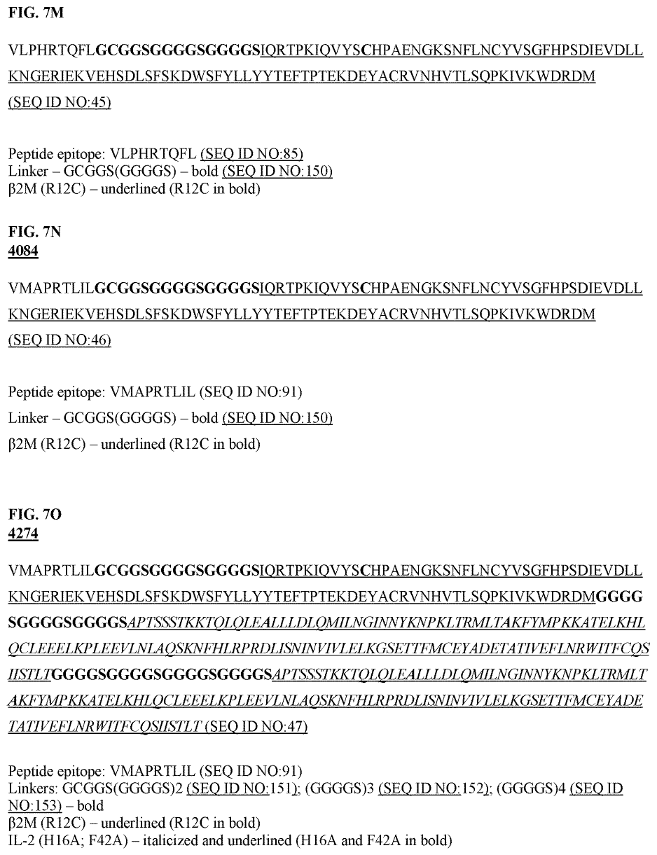

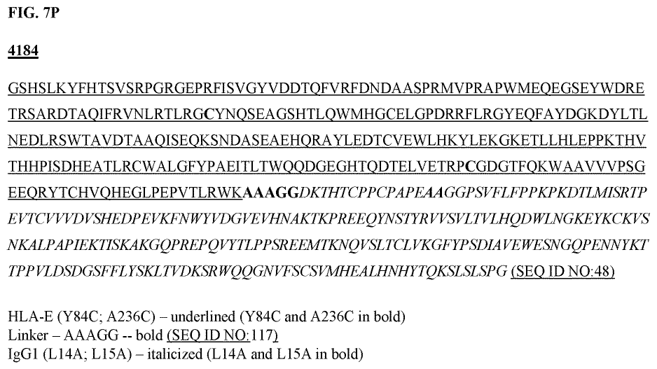

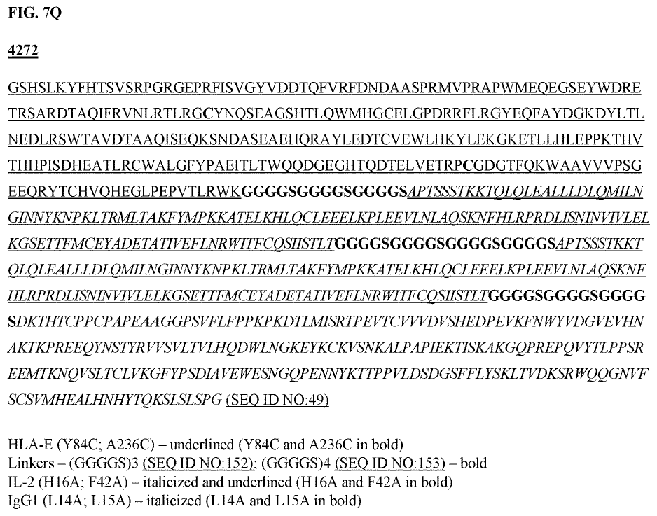

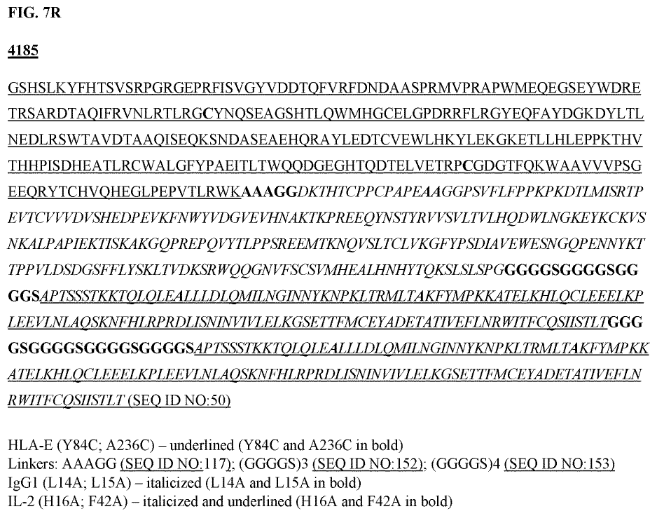

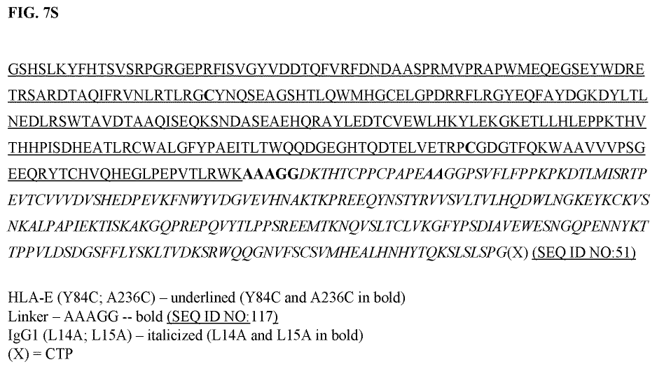

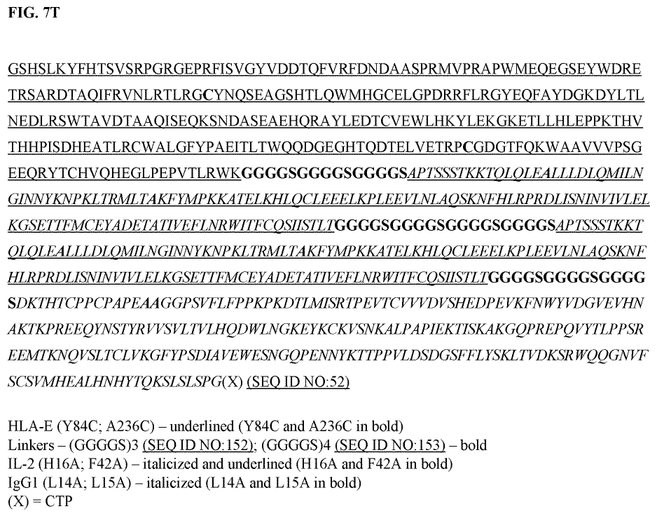

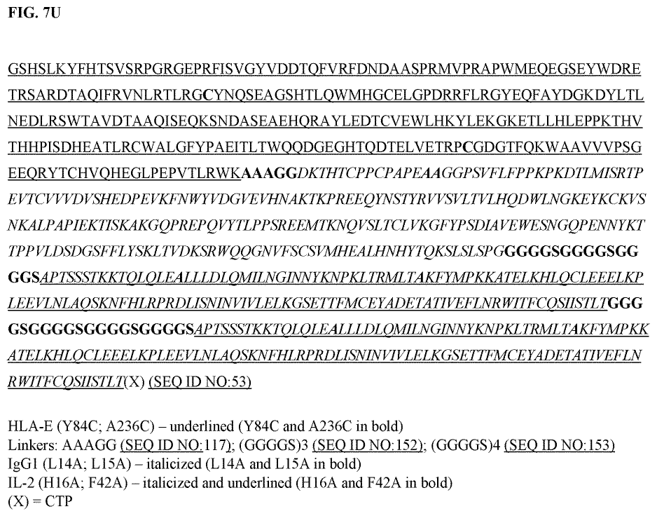

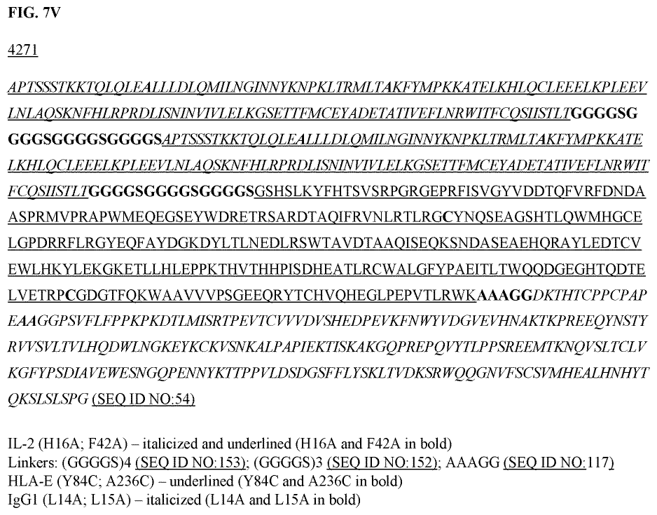

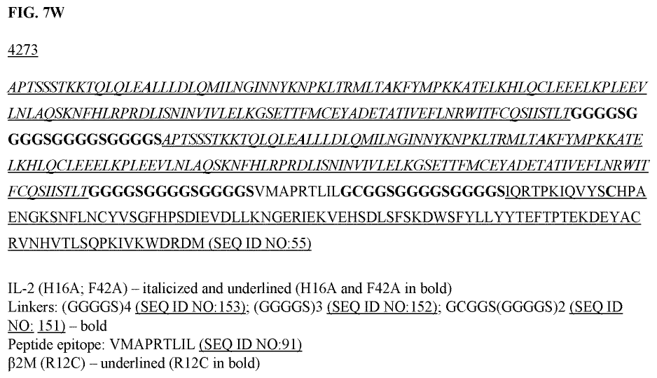

In some cases, a TMMP comprises: a) a first polypeptide comprising: i) an NKG2 peptide; ii) a peptide linker; and iii) a β2M polypeptide; and b) a second polypeptide comprising: i: an HLA-E heavy chain polypeptide; and ii) an Ig Fc polypeptide. In some cases, a TMMP comprises: a) a first polypeptide comprising: i) an NKG2 peptide; ii) a peptide linker; and iii) a β2M polypeptide; and b) a second polypeptide comprising: i: an HLA-E heavy chain polypeptide; ii) a MOD; and iii) an Ig Fc polypeptide. In some cases, a TMMP comprises: a) a first polypeptide comprising: i) an NKG2 peptide; ii) a peptide linker; and iii) a β2M polypeptide; and b) a second polypeptide comprising: i: an HLA-E polypeptide; ii) a CTP; and iii) an Ig Fc polypeptide. In some cases, a TMMP comprises: a) a first polypeptide comprising: i) an NKG2 peptide; ii) a peptide linker; and iii) a β2M polypeptide; and b) a second polypeptide comprising: i: an HLA-E heavy chain polypeptide; ii) a CTP; iii) a MOD; and iv) an Ig Fc polypeptide. For example, in some cases, the first polypeptide comprises an amino acid sequence as depicted in FIG. 7A, where (X) is an NKG2 peptide; and the second polypeptide comprises: i) an HLA-E heavy chain polypeptide; and ii) an Ig Fc polypeptide, e.g., as depicted in FIG. 7P. As another example, in some cases, the first polypeptide comprises an amino acid sequence as depicted in FIG. 7A, where (X) is an NKG2 peptide; and the second polypeptide comprises: i) an HLA-E heavy chain polypeptide; ii) one or more MODs; and iii) an IgFc polypeptide, e.g., as depicted in FIG. 7Q or FIG. 7R. As another example, in some cases, the first polypeptide comprises an amino acid sequence as depicted in FIG. 7A, where (X) is an NKG2 peptide; and the second polypeptide comprises: i) an HLA-E heavy chain polypeptide; ii) a CTP; and iii) an IgFc polypeptide, e.g., as depicted in FIG. 7S. As another example, in some cases, the first polypeptide comprises an amino acid sequence as depicted in FIG. 7A, where (X) is an NKG2 peptide; and the second polypeptide comprises: i) an HLA-E heavy chain polypeptide; ii) a CTP; iii) one or more MODs; and iv) an IgFc polypeptide, e.g., as depicted in FIG. 7T or FIG. 7U.

For example, in some cases, a TMMP comprises: i) a first polypeptide comprising an amino acid sequence having at least 95%, at least 97%, at least 98%, at least 99%, or 100%, amino acid sequence identity to the amino acid sequence depicted in any one of FIG. 7B-7M; and ii) a second polypeptide comprising an amino acid sequence having at least 95%, at least 97%, at least 98%, at least 99%, or 100%, amino acid sequence identity to the amino acid sequence depicted in FIG. 7P.

As another example, in some cases, a TMMP comprises: i) a first polypeptide comprising an amino acid sequence having at least 95%, at least 97%, at least 98%, at least 99%, or 100%, amino acid sequence identity to the amino acid sequence depicted in any one of FIG. 7B-7M; and ii) a second polypeptide comprising an amino acid sequence having at least 95%, at least 97%, at least 98%, at least 99%, or 100%, amino acid sequence identity to the amino acid sequence depicted in FIG. 7Q.

As another example, in some cases, a TMMP comprises: i) a first polypeptide comprising an amino acid sequence having at least 95%, at least 97%, at least 98%, at least 99%, or 100%, amino acid sequence identity to the amino acid sequence depicted in any one of FIG. 7B-7M; and ii) a second polypeptide comprising an amino acid sequence having at least 95%, at least 97%, at least 98%, at least 99%, or 100%, amino acid sequence identity to the amino acid sequence depicted in FIG. 7R.

As another example, in some cases, a TMMP comprises: i) a first polypeptide comprising an amino acid sequence having at least 95%, at least 97%, at least 98%, at least 99%, or 100%, amino acid sequence identity to the amino acid sequence depicted in any one of FIG. 7B-7M; and ii) a second polypeptide comprising an amino acid sequence having at least 95%, at least 97%, at least 98%, at least 99%, or 100%, amino acid sequence identity to the amino acid sequence depicted in FIG. 7S, where the CTP is an antibody (e.g., a scFv or a nanobody) specific for Her2, CD19, WT1, MUC1, BCMA, mesothelin, TROP-2, PRAME, MAGE-A4, or a claudin polypeptide.

As another example, in some cases, a TMMP comprises: i) a first polypeptide comprising an amino acid sequence having at least 95%, at least 97%, at least 98%, at least 99%, or 100%, amino acid sequence identity to the amino acid sequence depicted in any one of FIG. 7B-7M; and ii) a second polypeptide comprising an amino acid sequence having at least 95%, at least 97%, at least 98%, at least 99%, or 100%, amino acid sequence identity to the amino acid sequence depicted in FIG. 7T, where the CTP is an antibody (e.g., a scFv or a nanobody) specific for Her2, CD19, WT1, MUC1, BCMA, mesothelin, TROP-2, PRAME, MAGE-A4, or a claudin polypeptide.

As another example, in some cases, a TMMP comprises: i) a first polypeptide comprising an amino acid sequence having at least 95%, at least 97%, at least 98%, at least 99%, or 100%, amino acid sequence identity to the amino acid sequence depicted in any one of FIG. 7B-7M; and ii) a second polypeptide comprising an amino acid sequence having at least 95%, at least 97%, at least 98%, at least 99%, or 100%, amino acid sequence identity to the amino acid sequence depicted in FIG. 7U, where the CTP is an antibody (e.g., a scFv or a nanobody) specific for Her2, CD19, WT1, MUC1, BCMA, mesothelin, TROP-2, PRAME, MAGE-A4, or a claudin polypeptide.

As one example, in some cases, a TMMP comprises: i) a first polypeptide comprising the amino acid sequence depicted in FIG. 7N (construct 4084); and ii) a second polypeptide comprising the amino acid sequence depicted in FIG. 7P (construct 4184).

As another example in some cases, a TMMP comprises: i) a first polypeptide comprising the amino acid sequence depicted in FIG. 7N (construct 4084); and ii) a second polypeptide comprising the amino acid sequence depicted in FIG. 7Q (construct 4272).

As another example in some cases, a TMMP comprises: i) a first polypeptide comprising the amino acid sequence depicted in FIG. 7N (construct 4084); and ii) a second polypeptide comprising the amino acid sequence depicted in FIG. 7R (construct 4185).

As another example in some cases, a TMMP comprises: i) a first polypeptide comprising the amino acid sequence depicted in FIG. 7O (construct 4274); and ii) a second polypeptide comprising the amino acid sequence depicted in FIG. 7P (construct 4184).

As another example in some cases, a TMMP comprises: i) a first polypeptide comprising the amino acid sequence depicted in FIG. 7V (construct 4271); and ii) a second polypeptide comprising the amino acid sequence depicted in FIG. 7N (construct 4084).

As another example in some cases, a TMMP comprises: i) a first polypeptide comprising the amino acid sequence depicted in FIG. 7P (construct 4184); and ii) a second polypeptide comprising the amino acid sequence depicted in FIG. 7W (construct 4273).

### Dimerized TMMPs

A TMMP of the present disclosure can form dimers. The present disclosure thus provides a protein that is a dimerized TMMP comprising two TMMPs that are covalently linked to each other.

The covalent linkage of the dimer can be one or more disulfide bonds between an Ig Fc polypeptide in the first TMMP and an Ig Fc polypeptide in the second TMMP. As but one example, the Ig Fc can be a variant of a human IgG1 Fc polypeptide, which variant has a substantially reduced ability to effect complement-dependent cytotoxicity (CDC) or antibody-dependent cell cytotoxicity (ADCC). For example, the IgG1 Fc polypeptide can comprise substitutions of L14 and L15 (e.g., L14A and L15A substitutions).

When the TMMP comprises an Ig Fc polypeptide, the TMMP typically will self-assemble into a dimer by spontaneously forming one or more (e.g., two) disulfide bonds with the IgG1 Fc polypeptide of another TMMP. Thus, e.g., the Ig Fc polypeptides in the first TMMP and the second TMMP can be linked to one another by one or more (e.g., two) disulfide bonds. In many cases, the two TMMPs will be identical to one another in amino acid sequence and comprise Ig Fc polypeptides that spontaneously form one or more (e.g., two) disulfide bonds, thereby forming a dimerized TMMP that is a homodimer.

Accordingly, the present disclosure provides a protein comprising: a) a first TMMP; and b) a second TMMP, which optionally may be identical to the first TMMP, where the first and second TMMPs are covalently linked to one another by one or more (e.g., two) disulfide bonds. The covalent linkage can be one or more (e.g., two) disulfide bonds between an Ig Fc polypeptide in the first TMMP and an Ig Fc polypeptide in the second TMMP.

If desired, the Ig Fc polypeptides of each TMMP can comprise interspecific dimerization sequences, e.g., “Knob-in-Hole” sequences that permit two different TMMPs to selectively dimerize. Interspecific binding sequences favor formation of heterodimers with their cognate polypeptide sequence (i.e., the interspecific sequence and its counterpart interspecific sequence), particularly those based on Ig Fc sequence variants. Such interspecific polypeptide sequences include Knob-in-Hole, and Knob-in-Hole sequences that facilitate the formation of one or more disulfide bonds. For example, one interspecific binding pair comprises a T366Y and Y407T mutant pair in the CH3 domain interface of IgG1, or the corresponding residues of other immunoglobulins. See Ridgway et al., Protein Engineering 9:7, 617-621 (1996). A second interspecific binding pair involves the formation of a knob by a T366W substitution, and a hole by the triple substitutions T366S, L368A and Y407V on the complementary Ig Fc sequence. See Xu et al. mAbs 7:1, 231-242 (2015). Another interspecific binding pair has a first Fc polypeptide with Y349C, T366S, L368A, and Y407V substitutions and a second Ig Fc polypeptide with S354C, and T366W substitutions (disulfide bonds can form between the Y349C and the S354C). See, e.g., Brinkmann and Konthermann, mAbs 9:2, 182-212 (2015). Ig Fc polypeptide sequences, either with or without knob-in-hole modifications, can be stabilized by the formation of disulfide bonds between the Ig Fc polypeptides (e.g., the hinge region disulfide bonds). Thus, in some cases, a dimerized TMMP can be a heterodimer, comprising two TMMP chains that are not identical in amino acid sequence.

Interspecific dimerization sequences also may be employed to enable TMMPs to be linked to non-TMMP molecules that can provide additional functionality to the TMMP. For example, a TMMP could be linked to a molecule that comprise polypeptides (e.g., antibodies or binding fragments thereof such as scFvs) that bind to cancer-associated antigens, thereby enabling the TMMP to localize to tissues comprising the cancer-associated antigen.

### Nucleic Acids, Expression Vectors, Host Cells

The present disclosure provides nucleic acids comprising nucleotide sequences encoding a TMMP. In some cases, the individual polypeptide chains (the first polypeptide; the second polypeptide) of a TMMP are encoded in separate nucleic acids. In some cases, both polypeptide chains of a TMMP are encoded in a single nucleic acid. In some cases, a first nucleic acid comprises a nucleotide sequence encoding a first polypeptide of a TMMP; and a second nucleic acid comprises a nucleotide sequence encoding a second polypeptide of a TMMP. In some cases, single nucleic acid comprises a nucleotide sequence encoding a first polypeptide of a TMMP and a second polypeptide of a TMMP.

### Separate Nucleic Acids Encoding Individual Polypeptide Chains of a TMMP

The present disclosure provides plurality of nucleic acids comprising: a) a first nucleic acid comprising a nucleotide sequence encoding the first polypeptide of a TMMP; and b) a second nucleic acid comprising a nucleotide sequence encoding the second polypeptide of a TMMP. The present disclosure provides a composition comprising the plurality of nucleic acids. In some cases, nucleotide sequences encoding the separate polypeptide chains of a TMMP are operably linked to transcriptional control elements, e.g., promoters, such as promoters that are functional in a eukaryotic cell, where the promoter can be a constitutive promoter or an inducible promoter.

### Nucleic Acid Encoding Two or More Polypeptides Present in a Multimeric Polypeptide

The present disclosure provides a nucleic acid comprising nucleotide sequences encoding the first polypeptide and the second polypeptide of a TMMP. In some cases, the nucleotide sequences encoding the first polypeptide and the second polypeptide of a TMMP includes a proteolytically cleavable linker interposed between the nucleotide sequence encoding the first polypeptide and the nucleotide sequence encoding the second polypeptide. In some cases, the nucleotide sequences encoding the first polypeptide and the second polypeptide of a TMMP includes an internal ribosome entry site (IRES) interposed between the nucleotide sequence encoding the first polypeptide and the nucleotide sequence encoding the second polypeptide. In some cases, the nucleotide sequences encoding the first polypeptide and the second polypeptide of a TMMP includes a ribosome skipping signal (or cis-acting hydrolase element, CHYSEL) interposed between the nucleotide sequence encoding the first polypeptide and the nucleotide sequence encoding the second polypeptide. The nucleotide sequences encoding the first polypeptide and the second polypeptide can be operably linked to one or more transcriptional control elements, e.g., promoters, such as promoters that are functional in a eukaryotic cell, where the promoter can be a constitutive promoter or an inducible promoter.

### Recombinant Expression Vectors

The present disclosure provides recombinant expression vectors comprising nucleic acids as described above. Numerous suitable expression vectors are known to those of skill in the art, and many are commercially available.

### Genetically Modified Host Cells

The present disclosure provides a genetically modified host cell, where the host cell is genetically modified with one or more nucleic acids encoding the first and second polypeptides of a TMMP. The genetically modified host cell may be in vitro and can provide for production of the first and/or the second polypeptide of a TMMP.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

In some cases, the host cell is a mammalian cell that has been genetically modified such that it does not synthesize an endogenous $2M polypeptide. In some cases, the host cell is a mammalian cell that has been genetically modified such that it does not synthesize endogenous MHC Class I heavy chain. In some cases, the host cell is a mammalian cell that has been genetically modified such that it does not synthesize an endogenous $2M polypeptide and such that it does not synthesize endogenous MHC Class I heavy chain.

### Methods of Producing a TMMP

The present disclosure provides methods of producing a TMMP. The methods generally involve culturing, in a culture medium, a host cell that is genetically modified with one or more nucleic acids (e.g., one or more recombinant expression vectors) comprising nucleotide sequences encoding the first and second polypeptides of the TMMP; and isolating the TMMP from the genetically modified host cell and/or the culture medium. A host cell that is genetically modified with one or more nucleic acids (e.g., one or more recombinant expression vectors) comprising nucleotide sequences encoding the TMMP is also referred to as an “expression host.” As noted above, in some cases, the individual polypeptide chains (the first polypeptide and the second polypeptide) of a TMMP are encoded in separate nucleic acids (e.g., separate recombinant expression vectors). In some cases, both polypeptide chains of a TMMP are encoded in a single nucleic acid (e.g., a single recombinant expression vector).

Isolation of the TMMP from the expression host cell (e.g., from a lysate of the expression host cell) and/or the culture medium in which the host cell is cultured, can be carried out using standard methods of protein purification.

For example, a lysate may be prepared of the expression host and the lysate purified using high performance liquid chromatography (HPLC), exclusion chromatography, gel electrophoresis, affinity chromatography, or other purification technique. Alternatively, where the TMMP is secreted from the expression host cell into the culture medium, the TMMP can be purified from the culture medium using HPLC, exclusion chromatography, gel electrophoresis, affinity chromatography, or other purification technique. In some cases, the compositions which are used will comprise at least 80% by weight of the desired product (TMMP), at least about 85% by weight, at least about 95% by weight, or at least about 99.5% by weight, in relation to contaminants related to the method of preparation of the product and its purification. The percentages can be based upon total protein.

In some cases, e.g., where the TMMP comprises an affinity tag, the TMMP can be purified using an immobilized binding partner of the affinity tag.

### Compositions

The present disclosure provides compositions, including pharmaceutical compositions, comprising a TMMP, or a nucleic acid or recombinant expression vector comprising a nucleotide sequence encoding the TMMP, or a cell (e.g., a B cell or other blood cell) comprising such nucleic acids or recombinant expression vector, together with one or more pharmaceutically acceptable additives, a variety of which are known in the art and need not be discussed in detail herein. See, for example, the ninth (or latest) edition of Sheskey et al., “Handbook of Pharmaceutical Excipients” (2020), and/or the 23^{rd} (or latest) edition of “Remington: The Science and Practice of Pharmacy”, 23rd Ed. (2020).

In some cases, a treatment method comprises administering to an individual in need thereof one or more nucleic acids or recombinant expression vectors comprising nucleotide sequences encoding a TMMP. In some cases, the one or more nucleic acids or recombinant expression vectors are present in cells (e.g., B cells or other blood cells) that are administered to the individual, which cells are then capable of producing the TMMP in vivo.

In some cases, a subject pharmaceutical composition will be suitable for administration to a subject, e.g., will be sterile. For example, in some cases, a subject pharmaceutical composition will be suitable for administration to a human subject, e.g., where the composition is sterile and is substantially free of detectable pyrogens and/or other toxins, or where such detectable pyrogens and/or other toxins are present at a level within acceptable limits set by an applicable regulatory agency, e.g., the USF&DA. For example, compositions may include aqueous solution, powder form, granules, tablets, pills, suppositories, capsules, suspensions, sprays, and the like. The composition may be formulated according to the various routes of administration described below.

Where a TMMP is administered as an injectable (e.g. subcutaneously, intraperitoneally, intramuscularly, and/or intravenously) directly into a tissue, a formulation can be provided as a ready-to-use dosage form, or as non-aqueous form (e.g., a reconstitutable storage-stable powder) or aqueous form, such as liquid composed of pharmaceutically acceptable carriers and excipients. The protein-containing formulations may also be provided so as to enhance serum half-life of the TMMP following administration. For example, the TMMP may be provided in a liposome formulation, prepared as a colloid, or other conventional techniques for extending serum half-life. A variety of methods are available for preparing liposomes, as described in, e.g., Szoka et al. 1980 Ann. Rev. Biophys. Bioeng. 9:467, U.S. Pat. Nos. 4,235,871, 4,501,728 and 4,837,028. The preparations may also be provided in controlled release or slow-release forms.

TMMPs of this disclosure, e.g., a homodimer comprising two TMMPs, may be an aqueous liquid and administered via an intravenous infusion. In some cases, the pharmaceutical composition comprising the TMMP can be admixed with saline (e.g., 0.9% NaCl) prior to IV administration. Thus, the present disclosure provides a sterile composition comprising: a) a TMMP of the present disclosure; and b) saline (e.g., 0.9% NaCl). Alternatively, it may be administered neat via an intravenous infusion, i.e., without further dilution. Alternatively, the pharmaceutical composition may be formulated so as to be administered by injection.

Conventional and pharmaceutically acceptable routes of administration include intratumoral, peritumoral, intramuscular, intralymphatic, intratracheal, intracranial, subcutaneous, intradermal, topical application, intravenous, intraarterial, rectal, nasal, oral, and other enteral and parenteral routes of administration. As noted above, a pharmaceutical composition comprising a TMMP may be administered intravenously, but may also be administered by other routes that involve injection.

### Methods of Modulating NK Cell Activity

The present disclosure provides a method of substantially selectively modulating the activity of an NK cell, the method comprising contacting the T cell with a TMMP or dimerized TMMP, where contacting the NK cell with a TMMP or dimerized TMMP (e.g., a homodimer) selectively modulates the activity of the NK cell. In some cases, the contacting occurs in vitro. In some cases, the contacting occurs in vivo.

Where a TMMP or dimerized TMMP (e.g., a homodimer) includes a pMHC comprising a NKG2 peptide that promotes binding to the CD94/NKG2C receptor on an NK cell, the TMMP will activate the NK cell. Where a TMMP or dimerized TMMP (e.g., a homodimer) includes a pMHC comprising a NKG2 peptide that promotes binding to the CD94/NKG2A receptor on an NK cell, the TMMP will exert an inhibitory effect on the NK cell. Where a TMMP or dimerized TMMP (e.g., a homodimer) includes a pMHC comprising a NKG2 peptide that promotes binding to either the CD94/NKG2C and CD94/NKG2A receptors on an NK cell, the TMMP may exert an activating effect on some NK cells and inhibitory effect on other NK cells.

Where a TMMP or dimerized TMMP (e.g., a homodimer) includes a MOD for which the NK cell expresses a co-MOD, contacting the NK cell with the TMMP or dimerized TMMP (e.g., a homodimer) will provide an additional modulatory signal. For example, NK cells typically express IL-2Rβ and IL-2Rγ. Therefore, contacting the NK cell with a TMMP or dimerized TMMP that has a pMHC comprising an NKG2 peptide that promotes binding to the CD94/NKG2C receptor and that also comprises a MOD that is an activation MOD, e.g., IL-2, or a variant IL-2 polypeptide that binds to IL-2Rβ and/or IL2Rγ as described above, will provide a modulatory signal via the interaction between the IL-2 polypeptide and the IL-2Rβ and/or IL2Rγ. Such interaction may cause or contribute to proliferation of the NK cell, or may enhance the cytotoxic activity of the NK cell toward a cancer cell, e.g., by lowering the activation threshold of NK cells by tuning their ability to adhere to and engage with their targets. The IL-2 polypeptide MOD also may enhance the response of NK cells to interleukin-12 through up-regulation of the interleukin-12 receptor and STAT4. It is noted that some T cells also may have CD94/NKG2C receptors and thus the TMMP also may provide activating modulation to such T cells.

Conversely, contacting the NK cell with a TMMP or dimerized TMMP (e.g., a homodimer) that has a pMHC comprising an NKG2 peptide that promotes binding to the CD94/NKG2A receptor and includes a MOD that is an inhibitory MOD, e.g., PD-L1 or FasL, for which the NK cell has a receptor will provide an inhibitory signal via the interaction between the MOD and its co-MOD on the NK cell. Some T cells also may have CD94/NKG2A receptors and thus the TMMP also may provide inhibitory modulation to such T cells.

This disclosure thus provides a method of delivering a MOD substantially selectively to NK cells, the method comprising contacting a mixed population of cells with a TMMP or dimerized TMMP (e.g., a homodimer), where the mixed population of cells comprises NK cells and non-NK cells, where the contacting step delivers the one or more MODs present within the TMMP or dimerized TMMP (e.g., a homodimer) to the NK cells. In some cases, the population of cells is in vitro. In some cases, the population of cells is in vivo in an individual. In some cases, the method comprises administering the TMMP or dimerized TMMP (e.g., a homodimer) to the individual, e.g., an individual who has cancer. In some cases, the mixed population of cells is an in vitro population of cells obtained from an individual, e.g., an individual who has cancer, and the contacting step results in activation and/or proliferation of the NK cells, generating a population of activated and/or proliferated NK cells; in some of these instances, the method further comprises administering the population of activated and/or proliferated NK cells to the individual.

### Treatment Methods

This disclosure provides a method of treatment of cancer or an autoimmune disorder in an individual, the method comprising administering to the individual a pharmaceutical composition comprising an amount of i) a TMMP or dimerized TMMP (e.g., a homodimer), ii) one or more nucleic acids encoding the TMMP or dimerized TMMP, or iii) a B cell or other blood cell comprising nucleic acids encoding the TMMP, effective to treat the individual. (For ease of discussion below in the sections relating to administration and treatment of patients, including dosages, routes of administration, combination therapies and subjects suitable for treatment, references to a TMMP or dimerized TMMP are intended to also include one or more nucleic acids or one or more recombinant expression vectors encoding the TMMP or dimerized TMMP, or a B cell or other blood cell comprising nucleic acids encoding the TMMP or dimerized TMMP.) Also provided is a TMMP or dimerized TMMP (e.g., a homodimer) for use in a method of treatment of the human or non-human animal body. In some cases, a treatment method of this disclosure comprises administering to an individual in need thereof one or more nucleic acids or recombinant expression vectors comprising nucleotide sequences encoding a TMMP or dimerized TMMP (e.g., a homodimer). In some cases, the one or more nucleic acids or recombinant expression vectors are present in cells (e.g., B cells or other blood cells) that are administered to the individual, which cells are then capable of producing the TMMP or dimerized TMMP in vivo. In some cases, a treatment method of this disclosure comprises administering to an individual in need thereof one or more nucleic acids (e.g., mRNA molecules) comprising nucleotide sequences encoding a TMMP or dimerized TMMP, or a cell, e.g., a B cell or other blood cell such as a red blood cell comprising one or more of such nucleic acids. In some cases, a treatment method comprises administering to an individual in need thereof a TMMP or dimerized TMMP (e.g., a homodimer).

The present disclosure provides a method of treating a cancer in an individual, the method comprising administering to the individual an effective amount of a TMMP or a dimerized TMMP. Cancers that can be treated with a method include carcinomas, sarcomas, melanoma, leukemias, lymphomas and multiple myeloma. Cancers that can be treated with a method include solid tumors, and cancers that begin in blood-forming tissue, i.e., hematological cancers such as leukemias, lymphomas and multiple myeloma. Cancers that can be treated with a method include metastatic cancers. In some cases, the cancer is one that can be targeted with a CTP.

Cancers that that can be treated with a method include cancers that have escaped/evaded the immune system through HLA loss. Such cancers, which lack cell-surface expression of the class I major histocompatibility complex, thus can escape recognition by cancer-specific cytotoxic T cells. Because the anti-cancer effect of the TMMPs described herein does not depend on recognition of the cancer cells by cancer-specific cytotoxic T cells, but rather on activation of cytotoxic NK cells, HLA loss does not hinder the TMMPs from killing cancer cells.

Carcinomas that can treated by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

Sarcomas that can be treated by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be treated by a method disclosed herein include, but are not limited to, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

Leukemias that can be amenable to therapy by a method disclosed herein include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; non-Hodgkin's lymphoma, and the like.

Other cancers that can be treated according to the methods disclosed herein include atypical meningioma, islet cell carcinoma, medullary carcinoma of the thyroid, mesenchymoma, hepatocellular carcinoma, hepatoblastoma, clear cell carcinoma of the kidney, and neurofibroma mediastinum.

In some cases, an “effective amount” of a TMMP or a dimerized TMMP is an amount that, when administered in one or more doses to an individual in need thereof, either as a monotherapy or as part of a combination therapy (e.g., as part of a combination therapy with an immune checkpoint inhibitor, as discussed below), achieves one or more of the following: reduces the tumor mass/tumor volume in the individual; prevents a substantial increase (e.g., greater than 20%) in the overall tumor burden in the individual; reduces the overall tumor burden in the individual; reduces the number of cancer cells in the individual, including to substantially undetectable levels, as measured by the level of circulating tumor DNA (“ctDNA”) in the patient; and/or increases the survival time of the individual. The level of ctDNA can be determined using any known method; see, e.g., Cescon et al. (2020) Nature Cancer 1:276.

### Dosages

A suitable dosage of a TMMP or dimerized TMMP can be determined by an attending physician or other qualified medical personnel, based on various clinical factors. As is well known in the medical arts, dosages for any one patient depend upon many factors, including the patient's size, body surface area, age, the TMMP or dimerized TMMP (including the number and type(s) of MODs per TMP or dimerized TMP) or nucleic acid(s) (or a cell, e.g., a B cell or a blood cell such as a red blood cell comprising a nucleic acid(s) or recombinant expression vector(s)) to be administered, sex of the patient, time, and route of administration, general health, and other drugs being administered concurrently. Depending on these various factors, a TMMP or dimerized TMMP may be administered in amounts between 0.1 mg/kg body weight and 20 mg/kg body weight per dose, e.g. between 0.1 mg/kg body weight to 10 mg/kg body weight, e.g. between 0.5 mg/kg body weight to 5 mg/kg body weight, between 1 mg/kg body weight to 5 mg/kg body weight; between 5 mg/kg body weight to 10 mg/kg body weight; between 10 mg/kg body weight to 15 mg/kg body weight; between 15 mg/kg body weight to 20 mg/kg body weight, however, doses above this exemplary range are envisioned, especially considering the aforementioned factors. If the regimen is a continuous infusion, it can also be in the range of 1 g to 10 mg per kilogram of body weight per minute.

Persons of ordinary skill in the art can easily estimate repetition rates for dosing based on measured residence times and concentrations of the administered agent in bodily fluids or tissues. Following successful treatment, it may be desirable to have the patient undergo maintenance therapy to prevent the recurrence of the disease state, wherein a TMMP or dimerized TMMP is administered in maintenance doses, ranging from 0.1 mg/kg body weight to 1 mg/kg body weight, 1 mg/kg body weight to 5 mg/kg body weight, from 5 mg/kg body weight to 10 mg/kg body weight, or amounts exceeding 10 mg/kg of body weight.

Those of skill will readily appreciate that dose levels can vary as a function of the specific TMMP or dimerized TMMP, the severity of the symptoms and the susceptibility of the subject to side effects. Preferred dosages for a given compound are readily determinable by those of skill in the art by a variety of means.

In some cases, multiple doses of a TMMP or dimerized TMMPare administered. The frequency of administration of a TMMP or dimerized TMMP can vary depending on any of a variety of factors, e.g., severity of the symptoms, etc. For example, in some cases, a TMMP or dimerized TMMP is administered once per month, twice per month, three times per month, every other week (qow), once per week (qw), once every two weeks, once every three weeks, or once every four weeks. It also is possible to administer a TMMP more often than once per week. Where the TMMP or dimerized TMMP is administered intravenously, administration once every week, once every two weeks, once every three weeks or once every four weeks or once every month may be commonly employed at the beginning of treatment. When a TMMP or dimerized TMMP is administered with an immune checkpoint inhibitor (CPI), e.g., an anti-PD1 CPI such pembrolizumab, nivolumab, cemiplimab or durvalumab, then it may be desirable to administer the TMMP or dimerized TMMP on the same schedule as the CPI, e.g., once every three weeks.

The duration of administration of a TMMP or dimerized TMMP, a nucleic acid, or a recombinant expression vector (or a cell, e.g., a B cell or a blood cell such as a red blood cell comprising a nucleic acid or recombinant expression vector), e.g., the period of time over which a TMMP or dimerized TMMP, a nucleic acid, or a recombinant expression vector is administered, can vary, depending on any of a variety of factors, e.g., patient response, etc. For example, a TMMP or dimerized TMMP, a nucleic acid, or a recombinant expression vector (or a cell, e.g., a B cell or a blood cell such as a red blood cell comprising a nucleic acid or recombinant expression vector) can be administered over a period of time ranging from about one day to about one week, two weeks, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. The length of time may depend on whether the TMMP is administered in a neoadjuvant setting, in which case it may be administered only once or a few times, e.g., two, three or four times, for only a week or a few weeks before a main treatment such as surgery. In an adjuvant setting or a setting in which the patient is being treated for a cancer that has newly appeared or is recurrent or metastatic, the treatment duration may continue indefinitely or until the patient exhibits progressive disease, e.g., as determined by standard RECIST criteria.

### Routes of Administration

A TMMP or dimerized TMMP is administered to an individual using any available method and route suitable for systemic and localized routes of administration.

A TMMP or dimerized TMMP of this disclosure typically will be delivered via intravenous administration, but other conventional and pharmaceutically acceptable routes of administration may be used, including intratumoral, peritumoral, intramuscular, or subcutaneous. Other routes of administration also are possible, including intralymphatic, intratracheal, intracranial, intradermal, topical application, intraarterial, rectal, nasal, oral, and other enteral and parenteral routes of administration. Routes of administration may be combined, if desired, or adjusted depending upon the TMMP or dimerized TMMP and/or the desired effect. A TMMP or dimerized TMMP can be administered in a single dose or in multiple doses.

### Combination Therapy

A TMMP or dimerized TMMP can be administered to an individual in need thereof in combination with one or more additional therapeutic agents or therapeutic treatment. A suitable dosage amount of the TMMP or dimerized TMMP will be the same as the dosage amount for monotherapy with the TMMP or dimerized TMMP (described above) or may be less or more than the monotherapy dose.

A TMMP or dimerized TMMP can be administered to an individual in need thereof at the same time, or at different times, as the one or more additional therapeutic agent is administered.

Thus, for example, a treatment method can comprise co-administration of a TMMP or dimerized TMMP and at least one additional therapeutic agent. By “co-administration” is meant that both a TMMP or dimerized TMMP and at least one additional therapeutic agent are administered to an individual, although not necessarily at the same time, in order to achieve a therapeutic effect that is the result of having administered both the TMMP or dimerized TMMP and the at least one additional therapeutic agent. The administration of the TMMP or dimerized TMMP and the at least one additional therapeutic agent can be substantially simultaneous, e.g., the TMMP or dimerized TMMP can be administered to an individual within about 1 minute to about 24 hours (e.g., within about 1 minute, within about 5 minutes, within about 15 minutes, within about 30 minutes, within about 1 hour, within about 4 hours, within about 8 hours, within about 12 hours, or within about 24 hours) of administration of the at least one additional therapeutic agent. In some cases, a TMMP or dimerized TMMP is administered to an individual who is undergoing treatment with, or who has undergone treatment with, the at least one additional therapeutic agent. The administration of the TMMP or dimerized TMMP and the at least one additional therapeutic agent can occur at different times and/or at different frequencies.

### Combination Therapies in Cancer Treatment

A TMMP or dimerized TMMP can be administered to an individual in need thereof in combination with one or more additional therapeutic agents or therapeutic treatment. A suitable dosage of the TMMP or dimerized TMMP typically will be the same as the dosage amount for monotherapy with the TMMP (described above) or may be less or more than the monotherapy dose. Suitable additional therapeutic agents include, e.g.: i) an immune checkpoint inhibitor; ii) a cancer chemotherapeutic agent; and/or iii) one or more additional TMMPs or dimerized TMMPs. Suitable additional therapeutic treatments include, e.g., radiation, surgery (e.g., surgical resection of a tumor), and the like.

A TMMP or dimerized TMMP can be administered to an individual in need thereof at the same time, or at different times, as the one or more additional therapeutic agent is administered.

As noted above, a treatment method can comprise co-administration of a TMMP or dimerized TMMP and an immune checkpoint inhibitor such as an antibody specific for an immune checkpoint. By “co-administration” is meant that both a TMMP or dimerized TMMP and an antibody specific for an immune checkpoint are administered to an individual, although not necessarily at the same time, in order to achieve a therapeutic effect that is the result of having administered both the TMMP or dimerized TMMP and the immune checkpoint inhibitor. The administration of the TMMP or dimerized TMMP and the antibody specific for an immune checkpoint can be substantially simultaneous, e.g., the TMMP or dimerized TMMP can be administered to an individual within about 1 minute to about 24 hours (e.g., within about 1 minute, within about 5 minutes, within about 15 minutes, within about 30 minutes, within about 1 hour, within about 2 hours, within about 4 hours, within about 8 hours, within about 12 hours, or within about 24 hours) of administration of the antibody specific for an immune checkpoint. Alternatively, the TMMP or dimerized TMMP and immune checkpoint inhibitor can be administered on different schedules, including different days and different weeks, and different frequencies. In some cases, a TMMP or dimerized TMMP is administered to an individual who is undergoing treatment with, or who has undergone treatment with, an antibody specific for an immune checkpoint. In some cases, an individual who receives the combination therapy with a TMMP or dimerized TMMP and an anti-PD1 or anti-PD-L1 antibody will have a Combined Positive Score (CPS) of 1 or greater. In some cases, the individual receiving the combination therapy will have a CPS of 20 or greater. In some cases, the individual receiving the combination therapy will have a CPS of less than 20, e.g., from 0 to 19 or from 1 to 19.

Exemplary immune checkpoint inhibitors include inhibitors that target immune checkpoint polypeptide such as CD27, CD28, CD40, CD122, CD96, CD73, CD47, OX40, GITR, CSF1R, JAK, P13K delta, P13K gamma, TAM, arginase, CD137 (also known as 4-1BB), ICOS, A2AR, B7-H3, B7-H4, BTLA, CTLA-4, LAG3, TIM3, VISTA, CD96, TIGIT, CD122, PD-1, PD-L1 and PD-L2. In some cases, the immune checkpoint polypeptide is a stimulatory checkpoint molecule selected from CD27, CD28, CD40, ICOS, OX40, GITR, CD122, and CD137. In some cases, the immune checkpoint polypeptide is an inhibitory checkpoint molecule selected from A2AR, B7-H3, B7-H4, BTLA, CTLA-4, IDO, KIR, LAG3, PD-1, TIM3, CD96, TIGIT, and VISTA. In some cases, the immune checkpoint is an antibody that binds to CTLA-4, PD-1, PD-L1, or TIGIT.

In some cases, the immune checkpoint inhibitor is an antibody specific for an immune checkpoint. Suitable anti-immune checkpoint antibodies include, but are not limited to, nivolumab (Bristol-Myers Squibb), pembrolizumab (Merck), pidilizumab (Curetech), AMP-224 (GlaxoSmithKline/Amplimmune), MPDL3280A (Roche), MDX-1105 (Medarex, Inc./Bristol Myer Squibb), MEDI-4736 (Medimmune/AstraZeneca), arelumab (Merck Serono), ipilimumab (YERVOY, (Bristol-Myers Squibb), tremelimumab (Pfizer), pidilizumab (CureTech, Ltd.), IMP321 (Immutep S.A.), MGA271 (Macrogenics), BMS-986016 (Bristol-Meyers Squibb), lirilumab (Bristol-Myers Squibb), urelumab (Bristol-Meyers Squibb), PF-05082566 (Pfizer), IPH2101 (Innate Pharma/Bristol-Myers Squibb), MEDI-6469 (Medlmmune/AZ), CP-870,893 (Genentech), Mogamulizumab (Kyowa Hakko Kirin), Varlilumab (CelIDex Therapeutics), Avelumab (EMD Serono), Galiximab (Biogen Idec), AMP-514 (Amplimmune/AZ), AUNP 12 (Aurigene and Pierre Fabre), Indoximod (NewLink Genetics), NLG-919 (NewLink Genetics), INCB024360 (Incyte); KN035; and combinations thereof. For example, in some cases, the immune checkpoint inhibitor is an anti-PD-1 antibody. Suitable anti-PD-1 antibodies include, e.g., nivolumab, pembrolizumab (also known as MK-3475), pidilizumab, SHR-1210, PDR001, AMP-224, cemiplimab and durvalumab. In some cases, the anti-PD-1 monoclonal antibody is nivolumab, pembrolizumab, cemiplimab, durvalumab or PDR001. Suitable anti-PD1 antibodies are described in U.S. Patent Publication No. 2017/0044259. For pidilizumab, see, e.g., Rosenblatt et al. (2011) J. Immunother. 34:409-18. In some cases, the immune checkpoint inhibitor is an anti-CTLA-4 antibody. In some cases, the anti-CTLA-4 antibody is ipilimumab or tremelimumab. For tremelimumab, see, e.g., Ribas et al. (2013) J. Clin. Oncol. 31:616-22. In some cases, the immune checkpoint inhibitor is an anti-PD-L1 antibody. In some cases, the anti-PD-L1 monoclonal antibody is BMS-935559, MEDI4736, MPDL3280A (also known as RG7446), KN035, or MSB0010718C. In some cases, the anti-PD-L1 monoclonal antibody is MPDL3280A (atezolizumab) or MEDI4736 (durvalumab). For durvalumab, see, e.g., WO 2011/066389. For atezolizumab, see, e.g., U.S. Pat. No. 8,217,149. In some cases, the anti-TIGIT antibody is Tiragolumab (RG6058; MTIG7192A) (see US 2018/0186875. In some cases, the anti-TIGIT antibody is Vibostolimab (MK-7684) (see US 2018/0066055). In some cases, the anti-TIGIT antibody is Etigilimab (OMP-313M32).

Among such checkpoint inhibitors, antibodies to PD-1, PD-L1 and CTLA-4 are the most common, with at least nivolumab, tremelimumab, pembrolizumab, ipilimumab, cemiplimab, atezolizumab, avelumab, tisleizumab and durvalumab having been approved by the FDA and/or regulatory agencies outside of the U.S. The TMMPs and dimerized TMMPs of this disclosure also may be co-administered with combinations of checkpoint inhibitors, e.g., a combination of (i) an antibody to PD-1 or PD-L1, and (ii) an antibody to CTLA-4, or a combination of (i) an antibody to PD-1 or PD-L1, and (ii) an antibody to TIGIT.

### Subjects Suitable for Treatment

Subjects suitable for treatment with a method of this disclosure include individuals who have cancer, including individuals who have been diagnosed as having cancer, individuals who have been treated for cancer but who failed to respond to the treatment, and individuals who have been treated for cancer and who initially responded but subsequently became refractory to the treatment.

In some cases, the subject is an individual who is undergoing treatment with an immune checkpoint inhibitor. In some cases, the subject is an individual who has undergone treatment with an immune checkpoint inhibitor, but whose disease has progressed despite having received such treatment. In some cases, the subject is an individual who is undergoing treatment with, or who has undergone treatment with, a cancer chemotherapeutic agent. In some cases, the subject is an individual who is preparing to undergo treatment with, is undergoing treatment with, or who has undergone treatment with, an immune checkpoint inhibitor. In some cases, the subject is an individual who is preparing to undergo treatment with, is undergoing treatment with, or who has undergone treatment with, a cancer chemotherapeutic agent, radiation treatment, surgery, and/or treatment with another therapeutic agent.

Subjects suitable for treatment with a method of this disclosure also include individuals who have a condition associated with an autoimmune disorder, a metabolic disorder, or GVHD, including individuals who have been diagnosed as having an autoimmune disorder, a metabolic disorder, or GVHD, individuals who have been treated for such disorders but who failed to respond to the treatment, and individuals who have been treated for such disorders and who initially responded but subsequently became refractory to the treatment.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A T-cell modulatory multimeric polypeptide (TMMP) comprising at least one heterodimer comprising:
a) a first polypeptide comprising, from N-terminus to C-terminus:
   i) an NKG2 peptide having a length of from about 8 amino acids to about 12 amino acids that, when present in peptide-major histocompatibility complex (pMHC) comprising the NKG2 peptide, an HLA-E heavy chain polypeptide and a beta-2 microglobulin (β2M) polypeptide, presents an epitope to a CD94/NKG2A receptor and/or a CD94/NKG2C receptor,
   ii) an optional peptide linker, and
   iii) a β2M polypeptide; and
b) a second polypeptide comprising:
   i) an HLA-E heavy chain polypeptide,
   ii) an optional peptide linker, and
   iii) an immunoglobulin (Ig) Fc polypeptide.

Aspect 2. The TMMP of aspect 1, wherein the HLA-E polypeptide comprises an amino acid sequence having at least 90% amino acid sequence identity to the amino acid sequence depicted in FIG. 1A or FIG. 2A.

Aspect 3. The TMMP of any one of aspects 1-10, wherein the β2M polypeptide comprises an amino acid sequence having at least 95% amino acid sequence identity to the β2M amino acid sequence depicted in FIG. 3A.

Aspect 4. The TMMP of aspect 1 or aspect 2, wherein the TMMP comprises a disulfide bond that joins a Cys residue in the β2M polypeptide to a Cys residue in the HLA-E heavy chain polypeptide.

Aspect 5. The TMMP of aspect 2, wherein the β2M polypeptide comprises a Cys at amino acid 12, wherein the HLA-E polypeptide comprises a Cys at amino acid 236, and wherein the heterodimer comprises a disulfide bond formed between the Cys at amino acid 12 of the β2M polypeptide and the Cys at amino acid 236 of the HLA-E polypeptide.

Aspect 6. The TMMP of any one of aspects 1-4, wherein:
a) the first polypeptide comprises a peptide linker between the NKG2 peptide and the β2M polypeptide, wherein the peptide linker comprises a Cys,
b) the HLA-E polypeptide comprises a Cys at any one of amino acids 135-143, based on the numbering of the HLA-E polypeptide depicted in FIG. 1A or FIG. 2A, and wherein amino acid 84, based on the numbering of the HLA-E polypeptide depicted in FIG. 1A or FIG. 2A, is other than Cys; and
wherein the heterodimer comprises a disulfide bond between the Cys present in the peptide linker and the Cys at any one of amino acids 135-143 of the HLA-E polypeptide.

Aspect 7. The TMMP of aspect 5, wherein the HLA-E heavy chain polypeptide comprises a Cys at amino acid 138, 139, or 140 based on the numbering of the HLA-E polypeptide depicted in FIG. 1A or FIG. 2A.

Aspect 8. The TMMP of aspect 6, wherein the HLA-E heavy chain polypeptide comprises a Cys at amino acid 139 based on the numbering of the HLA-E polypeptide depicted in FIG. 1A or FIG. 2A.

Aspect 9. The TMMP of any one of aspects 1-8, wherein:
a) the first polypeptide comprises a peptide linker between the NKG2 peptide and the β2M polypeptide, wherein the peptide linker comprises a Cys,
b) the HLA-E polypeptide comprises a Cys at amino acid 84; and
wherein the heterodimer comprises a disulfide bond between the Cys present in the peptide linker and the Cys at amino acid 84 of the HLA-E polypeptide.

Aspect 10. The TMMP of aspect 9, wherein the peptide linker between the NKG2 peptide and the β2M polypeptide comprises the sequence CGGGS(GGGGS)n (SEQ ID NO: 126), GCGGS(GGGGS)n (SEQ ID NO: 127), or GGCGS(GGGGS)n (SEQ ID NO:128), wherein n is an integer from 1-10.

Aspect 11. The TMMP of any one of aspects 1-9, wherein the β2M polypeptide comprises a Cys at amino acid 12, wherein the HLA-E polypeptide comprises a Cys at amino acid 236, and wherein the heterodimer comprises a disulfide bond formed between the Cys at amino acid 12 of the β2M polypeptide and the Cys at amino acid 236 of the HLA-E polypeptide.

Aspect 12. The TMMP of any one of aspects 1-11, wherein the Ig Fc polypeptide comprises an amino acid sequence having at least 95% amino acid sequence identity to the amino acid sequence depicted in FIG. 4A.

Aspect 13. The TMMP of aspect 12, wherein the Ig Fc polypeptide is a variant Ig Fc polypeptide comprises one or more amino acid substitutions that reduce or substantially eliminate antibody-dependent cell-mediated cytotoxicity (ADCC) and/or complement-dependent cytotoxicity (CDC).

Aspect 14. The TMMP of aspect 13, wherein the Ig Fc comprises a substitution of L14 and L15, based on numbering of the amino acid sequence depicted in FIG. 4A.

Aspect 15. The TMMP of aspect 14, wherein the Ig Fc comprises an Ala at amino acid 14 and an Ala at amino acid 15, based on numbering of the amino acid sequence depicted in FIG. 4A.

Aspect 16. The TMMP of any one of aspects 1-15, wherein the TMMP exhibits preferential binding to a CD94/NKG2C receptor compared to binding of the TMMP to a CD94/NKG2A receptor.

Aspect 17. The TMMP of any one of aspects 1-15, wherein the TMMP exhibits preferential binding to a CD94/NKG2A receptor compared to binding of the TMMP to a CD94/NKG2C receptor.

Aspect 18. The TMMP of any one of aspects 1-15, wherein the NKG2 peptide comprises an amino acid sequence selected from the group consisting of QMPSRSLLF (SEQ ID NO:73), TLPKRGLFL (SEQ ID NO:74), TGPWRSLWI (SEQ ID NO:75), ILTDRSLWL (SEQ ID NO:76), VNPGRSLFL (SEQ ID NO:77), VMAPRTLFL (SEQ ID NO:78), TAPARTMFL (SEQ ID NO:79), TLPERTLYL (SEQ ID NO:80), VMPPRTLLL (SEQ ID NO:81), VMPGRTLCF (SEQ ID NO:82), RMPPRSVLL (SEQ ID NO:83), NMPARTVLF (SEQ ID NO:84), and VLPHRTQFL (SEQ ID NO:85).

Aspect 19. The TMMP of any one of aspects 1-18, wherein:
a) the first polypeptide comprises, in order from N-terminus to C-terminus:
   i) the NKG2 peptide;
   ii) a peptide linker; and
   iii) the β2M polypeptide; and
b) the second polypeptide comprises, in order from N-terminus to C-terminus
   i) the HLA-E polypeptide;
   ii) a peptide linker; and
   iii) the Ig Fc polypeptide.

Aspect 20. The TMMP of any one of aspects 1-18, wherein the second polypeptide further comprises one or more immunomodulatory polypeptides, and wherein when the TMMP comprises more than one immunomodulatory polypeptide, one or more linkers may be interposed between the more than one immunomodulatory polypeptides.

Aspect 21. The TMMP of aspect 19, wherein at least one of the one or more immunomodulatory polypeptides is a wild-type or variant of an activating immunomodulatory polypeptide, optionally selected from IL-2, a 4-1BBL, CD80, CD86, and combinations thereof, and optionally wherein at least one immunomodulatory polypeptide is a variant immunomodulatory polypeptide that exhibits reduced affinity to a cognate costimulatory polypeptide compared to the affinity of a corresponding wild-type immunomodulatory polypeptide for the cognate costimulatory polypeptide.

Aspect 22. The TMMP of aspect 20, wherein the at least one immunomodulatory polypeptide is a variant of IL-2 that exhibits decreased binding affinity for IL-2Rα and IL-2Rβ, optionally wherein the variant IL-2 polypeptide comprises an amino acid other than histidine at position 16 and an amino acid other than phenylalanine at position 42.

Aspect 23. The TMMP of aspect 21, wherein the immunomodulatory polypeptide is a variant IL-2 polypeptide comprising an amino acid at position 16 other than His and an amino acid at position 42 other than Phe.

Aspect 24. The TMMP of aspect 21, wherein the at least one immunomodulatory polypeptide comprises i) an H16A substitution and an F42A substitution; ii) an H16T substitution and an F42A substitution, iii) an H16D and F42A substitution, or an H16E substitution and an F42A substitution, or iv) an H16E substitution and an F42A substitution.

Aspect 25. The TMMP of any one of aspects 20-24, wherein:
a) the first polypeptide comprises, in order from N-terminus to C-terminus:
   i) the NKG2 peptide;
   ii) a peptide linker; and
   iii) the β2M polypeptide; and
b) the second polypeptide comprises, in order from N-terminus to C-terminus
   i) the HLA-E polypeptide;
   ii) a peptide linker;
   iii) the Ig Fc polypeptide;
   iv) a peptide linker; and
   v) the one or more immunomodulatory polypeptides.

Aspect 26. The TMMP of any one of aspects 20-24, wherein:
a) the first polypeptide comprises, in order from N-terminus to C-terminus:
   i) the NKG2 peptide;
   ii) a peptide linker; and
   iii) the β2M polypeptide; and
b) the second polypeptide comprises, in order from N-terminus to C-terminus
   i) the one or more immunomodulatory polypeptides;
   ii) a peptide linker;
   iii) the HLA-E polypeptide;
   iv) a peptide linker; and
   v) the Ig Fc polypeptide.

Aspect 27. The TMMP of any one of aspects 1-19, wherein the second polypeptide further comprises a cancer-targeting polypeptide (CTP).

Aspect 28. The TMMP of aspect 27, wherein the CTP is an antibody specific for a cancer-associated antigen on the surface of a cancer cell.

Aspect 29. The TMMP of aspect 28, wherein the antibody is specific for Her2, CD19, WT1, MUC1, BCMA, mesothelin, TROP-2, PRAME, MAGE-A4, or a claudin polypeptide.

Aspect 30. The TMMP of aspect 28 or aspect 29, wherein the antibody is a single-chain Fv (scFv) polypeptide.

Aspect 31. The TMMP of aspect 28 or aspect 29, wherein the antibody is a nanobody.

Aspect 32. The TMMP of any one of aspects 27-31, wherein:
a) the first polypeptide comprises, in order from N-terminus to C-terminus:
   i) the NKG2 peptide;
   ii) a peptide linker; and
   iii) the β2M polypeptide; and
b) the second polypeptide comprises, in order from N-terminus to C-terminus
   i) the HLA-E polypeptide;
   ii) a peptide linker;
   iii) the Ig Fc polypeptide;
   iv) a peptide linker; and
   v) the CTP.

Aspect 33. The TMMP of any one of aspects 27-31, wherein:
a) the first polypeptide comprises, in order from N-terminus to C-terminus:
   i) the NKG2 peptide;
   ii) a peptide linker; and
   iii) the β2M polypeptide; and
b) the second polypeptide comprises, in order from N-terminus to C-terminus
   i) the CTP;
   ii) a peptide linker;
   iii) the HLA-E polypeptide;
   iv) a peptide linker; and
   v) the Ig Fc polypeptide;

Aspect 34. The TMMP of any one of aspects 27-33, wherein the second polypeptide further comprises one or more immunomodulatory polypeptides, and wherein when the TMMP comprises more than one immunomodulatory polypeptide, one or more linkers may be interposed between the more than one immunomodulatory polypeptides.

Aspect 35. The TMMP of aspect 34, wherein at least one of the one or more immunomodulatory polypeptides is a wild-type or variant of an activating immunomodulatory polypeptide, optionally selected from IL-2, a 4-1BBL, CD80, CD86, and combinations thereof, and optionally wherein at least one immunomodulatory polypeptide is a variant immunomodulatory polypeptide that exhibits reduced affinity to a cognate costimulatory polypeptide compared to the affinity of a corresponding wild-type immunomodulatory polypeptide for the cognate costimulatory polypeptide.

Aspect 36. The TMMP of aspect 35, wherein the at least one immunomodulatory polypeptide is a variant of IL-2 that exhibits decreased binding affinity for IL-2Rα and IL-2Rβ, optionally wherein the variant IL-2 polypeptide comprises an amino acid other than histidine at position 16 and an amino acid other than phenylalanine at position 42.

Aspect 37. The TMMP of aspect 36, wherein the immunomodulatory polypeptide is a variant IL-2 polypeptide comprising an amino acid at position 16 other than His and an amino acid at position 42 other than Phe.

Aspect 38. The TMMP of aspect 37, wherein the at least one immunomodulatory polypeptide comprises i) an H16A substitution and an F42A substitution; ii) an H16T substitution and an F42A substitution, iii) an H16D and F42A substitution, or an H16E substitution and an F42A substitution, or iv) an H16E substitution and an F42A substitution.

Aspect 39. The TMMP of any one of aspects 34-38, wherein:
a) the first polypeptide comprises, in order from N-terminus to C-terminus:
   i) the NKG2 peptide;
   ii) a peptide linker; and
   iii) the β2M polypeptide; and
b) the second polypeptide comprises, in order from N-terminus to C-terminus
   i) the HLA-E polypeptide;
   ii) a peptide linker;
   iii) the Ig Fc polypeptide;
   iv) a peptide linker;
   v) the one or more immunomodulatory polypeptides;
   vi) a peptide linker; and
   vii) the CTP.

Aspect 40. The TMMP of any one of aspects 20-24, wherein:
a) the first polypeptide comprises, in order from N-terminus to C-terminus:
   i) the NKG2 peptide;
   ii) a peptide linker; and
   iii) the β2M polypeptide; and
b) the second polypeptide comprises, in order from N-terminus to C-terminus
   i) the one or more immunomodulatory polypeptides;
   ii) a peptide linker;
   iii) the HLA-E polypeptide;
   iv) a peptide linker;
   v) the Ig Fc polypeptide;
   vi) a peptide linker; and
   vii) the CTP.

Aspect 41. A homodimer comprising two TMMPs of any one of aspects 1-40.

Aspect 42. A nucleic acid comprising a nucleotide sequence encoding the first and the second polypeptides of any one of aspects 1-40.

Aspect 43. A recombinant expression vector comprising the nucleic acid of aspect 42.

Aspect 44. An in vitro host cell genetically modified with the recombinant expression vector of aspect 47.

Aspect 45. A plurality of nucleic acids, the plurality comprising:
a) a first nucleic acid comprising a nucleotide sequence encoding the first polypeptide of any one of aspects 1-40; and
b) a second nucleic acid comprising a nucleotide sequence encoding the second polypeptide of any one of aspects 1-40.

Aspect 46. A plurality of recombinant expression vectors, the plurality comprising:
a) a first recombinant expression vector comprising the first nucleic acid of aspect 45; and
b) a second recombinant expression comprising the second nucleic acid of aspect 45.

Aspect 47. An in vitro host cell genetically modified with the plurality of recombinant expression vectors of aspect 46.

Aspect 48. A method of producing a TMMP of any one of aspects 1-40, the method comprising culturing the host cell of aspect 44 or aspect 47 in a culture medium, wherein the host cell produces the TMMP.

Aspect 49. A pharmaceutical composition comprising the TMMP of any one of aspects 1-40.

Aspect 50. A method of treating cancer in an individual, the method comprising administering to the individual an effective amount of the TMMP of any one of aspects 1-40, or the composition of aspect 49, wherein the TMMP selectively activates an NKG2C⁺ NK cell in the individual.

Aspect 51. A method of treating an autoimmune disorder in an individual, the method comprising administering to the individual an effective amount of the TMMP of any one of aspects 1-40, or the composition of aspect 49, wherein the TMMP selectively inhibits an NKG2A⁺ NK cell in the individual.

### EXAMPLES

The following examples are put forth so as to provide those of ordinary skill in the art with a complete disclosure and description of how to make and use the present disclosure, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

TMMPs comprising the peptide VMAPRTLIL, an HLA-E heavy chain polypeptide, a β2M polypeptide, and an Ig Fc polypeptide, with or without 2 copies of a variant IL-2, were generated. Features of the constructs are shown in Table 3, below.

TABLE 3

| TMMP | Epitope | Position of IL-2 |
|---|---|---|
| 4184-4084 | VMAPRTLIL | (no IL-2) |
| 4272-4084 | VMAPRTLIL | 2xIL-2 in position 2 |
| 4185-4084 | VMAPRTLIL | 2xIL-2 in position 3 |
| 4184-4274 | VMAPRTLIL | 2xIL-2 in position 5 |
| 4271-4084 | VMAPRTLIL | 2xIL-2 in position 1 |
| 4184-4273 | VMAPRTLIL | 2xIL-2 in position 4 |

The amino acid sequence of the polypeptide chain 4084 is provided in FIG. 7N. The 4084 polypeptide comprises: i) VMAPRTLIL as the peptide epitope; ii) a “G2C” linker having the amino acid sequence GCGGS(GGGGS)2 (SEQ ID NO: 127); and iii) a β2M polypeptide comprising a Cys at amino acid 12 (β2M(R12C)). The amino acid sequence of the polypeptide chain 4274 is provided in FIG. 7O. The 4274 polypeptide comprises: i) VMAPRTLIL as the peptide epitope; ii) a “G2C” linker having the amino acid sequence GCGGS(GGGGS)2 (SEQ ID NO: 127); iii) a β2M polypeptide comprising a Cys at amino acid 12 (β2M(R12C)); iv) a (GGGGS)3 linker; v) a first variant IL-2 comprising H16A and F42A substitutions; vi) a (GGGS)4 linker; and vii) a second variant IL-2 comprising H16A and F42A substitutions. The amino acid sequence of the polypeptide chain 4273 is provided in FIG. 7W. The 4273 polypeptide comprise: i) a first variant IL-2 comprising H16A and F42A substitutions; ii) a (GGGS)4 linker; iii) a second variant IL-2 comprising H16A and F42A substitutions; iv) a (GGGGS)3 linker; v) VMAPRTLIL as the peptide epitope; vi) a “G2C” linker having the amino acid sequence GCGGS(GGGGS)2 (SEQ ID NO: 127); and vii) a β2M polypeptide comprising a Cys at amino acid 12 (β2M(R12C)).

The amino acid sequence of the polypeptide chain 4184 is provided in FIG. 7P. The 4184 polypeptide comprises: i) an HLA-E (Y84C; A236C) polypeptide; ii) an AAAGG linker; and iii) an IgG Fc polypeptide with L14A and L15A substitutions. The amino acid sequence of the polypeptide chain 4272 is provided in FIG. 7Q. The 4272 polypeptide comprises: i) an HLA-E (Y84C; A236C) polypeptide; ii) a (GGGS)3 linker; ii) a first variant IL-2 comprising H16A and F42A substitutions; iii) a (GGGS)3 linker; iv) a second variant IL-2 comprising H16A and F42A substitutions; v) a (GGGS)3 linker; and vi) an IgG Fc polypeptide with L14A and L15A substitutions. The amino acid sequence of the polypeptide chain 4185 is provided in FIG. 7R. The 4185 polypeptide comprises: an HLA-E (Y84C; A236C) polypeptide; ii) an AAAGG linker; iii) an IgG Fc polypeptide with L14A and L15A substitutions; iv) a (GGGGS)3 linker; v) a first variant IL-2 comprising H16A and F42A substitutions; vi) a (GGGS)4 linker; and vii) a second variant IL-2 comprising H16A and F42A substitutions. The amino acid sequence of the polypeptide chain 4271 is provided in FIG. 7V. The 4271 polypeptide comprises: i) a first variant IL-2 comprising H16A and F42A substitutions; ii) a (GGGS)4 linker; iii) a second variant IL-2 comprising H16A and F42A substitutions; iv) a (GGGGS)3 linker; v) an HLA-E (Y84C; A236C) polypeptide; vi) an AAAGG linker; and vii) an IgG Fc polypeptide with L14A and L15A substitutions.

The TMMPs shown in Table 4 were produced in CHO cells and purified on Protein A columns. The production level (“ProA expression (mg/L)”) and % monomer are shown in Table 5, below.

TABLE 5

| | ProA expression | % | MOD |
|---|---|---|---|
| TMMP | (mg/L) | monomer | position |
| 4184-4084 | 65 | 87 | (no MOD) |
| 4272-4084 | 17 | 57 | 2 |
| 4185-4084 | 40 | 81 | 3 |
| 4185-4084 | 58 | 90 | 3 |
| 4184-4274 | 60 | 68 | 5 |
| 4271-4084 | 91 | 72 | 1 |
| 4184-4273 | 73 | 33 | 4 |

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the claims appended hereto.

What is claimed is:

1. A T-cell modulatory multimeric polypeptide (TMMP) comprising at least one heterodimer comprising:
a) a first polypeptide comprising, from N-terminus to C-terminus:
i) an NKG2 peptide having a length of from about 8 amino acids to about 12 amino acids that, when present in peptide-major histocompatibility complex (pMHC) comprising the NKG2 peptide, an HLA-E heavy chain polypeptide and a beta-2 microglobulin (β2M) polypeptide, presents an epitope to a CD94/NKG2A receptor and/or a CD94/NKG2C receptor,
ii) an optional peptide linker, and
iii) a β2M polypeptide; and
b) a second polypeptide comprising:
i) an HLA-E heavy chain polypeptide,
ii) an optional peptide linker, and
iii) an immunoglobulin (Ig) Fc polypeptide,
wherein:
c) the first polypeptide comprises a peptide linker between the NKG2 peptide and the β2M polypeptide, wherein the peptide linker comprises a Cys,
d) the HLA-E polypeptide comprises a Cys at any one of amino acids 135-143, based on the numbering of the HLA-E polypeptide depicted in FIG. 1A or FIG. 2A, and wherein amino acid 84, based on the numbering of the HLA-E polypeptide depicted in FIG. 1A or FIG. 2A, is other than Cys; and
wherein the heterodimer comprises a disulfide bond between the Cys present in the peptide linker and the Cys at any one of amino acids 135-143 of the HLA-E polypeptide.

2. The TMMP of claim 1, wherein the HLA-E polypeptide comprises an amino acid sequence having at least 90% amino acid sequence identity to the amino acid sequence depicted in FIG. 1A or FIG. 2A.

3. The TMMP of claim 1 or claim 2, wherein the β2M polypeptide comprises an amino acid sequence having at least 95% amino acid sequence identity to the β2M amino acid sequence depicted in FIG. 3A.

4. The TMMP of claim 1 or claim 2, wherein the TMMP comprises a disulfide bond that joins a Cys residue in the β2M polypeptide to a Cys residue in the HLA-E heavy chain polypeptide.

5. The TMMP of claim 2, wherein the β2M polypeptide comprises a Cys at amino acid 12, wherein the HLA-E polypeptide comprises a Cys at amino acid 236, and wherein the heterodimer comprises a disulfide bond formed between the Cys at amino acid 12 of the β2M polypeptide and the Cys at amino acid 236 of the HLA-E polypeptide.

6. The TMMP of claim 5, wherein the HLA-E heavy chain polypeptide comprises a Cys at amino acid 138, 139, or 140 based on the numbering of the HLA-E polypeptide depicted in FIG. 1A or FIG. 2A.

7. The TMMP of claim 6, wherein the HLA-E heavy chain polypeptide comprises a Cys at amino acid 139 based on the numbering of the HLA-E polypeptide depicted in FIG. 1A or FIG. 2A.

8. The TMMP of any one of claims 1-7, wherein:
a) the first polypeptide comprises a peptide linker between the NKG2 peptide and the β2M polypeptide, wherein the peptide linker comprises a Cys,
b) the HLA-E polypeptide comprises a Cys at amino acid 84; and
wherein the heterodimer comprises a disulfide bond between the Cys present in the peptide linker and the Cys at amino acid 84 of the HLA-E polypeptide.

9. The TMMP of claim 8, wherein the peptide linker between the NKG2 peptide and the β2M polypeptide comprises the sequence CGGGS(GGGGS)ₙ, GCGGS(GGGGS)ₙ, or GGCGS(GGGGS)ₙ, wherein n is an integer from 1-10.

10. The TMMP of any one of claims 1-9, wherein the β2M polypeptide comprises a Cys at amino acid 12, wherein the HLA-E polypeptide comprises a Cys at amino acid 236, and wherein the heterodimer comprises a disulfide bond formed between the Cys at amino acid 12 of the β2M polypeptide and the Cys at amino acid 236 of the HLA-E polypeptide.

11. The TMMP of any one of claims 1-10, wherein the Ig Fc polypeptide comprises an amino acid sequence having at least 95% amino acid sequence identity to the amino acid sequence depicted in FIG. 4A.

12. The TMMP of claim 11, wherein the Ig Fc polypeptide is a variant Ig Fc polypeptide comprises one or more amino acid substitutions that reduce or substantially eliminate antibody-dependent cell-mediated cytotoxicity (ADCC) and/or complement-dependent cytotoxicity (CDC).

13. The TMMP of claim 12, wherein the Ig Fc comprises a substitution of L14 and L15, based on numbering of the amino acid sequence depicted in FIG. 4A.

14. The TMMP of claim 13, wherein the Ig Fc comprises an Ala at amino acid 14 and an Ala at amino acid 15, based on numbering of the amino acid sequence depicted in FIG. 4A.

15. The TMMP of any one of claims 1-14, wherein the TMMP exhibits preferential binding to a CD94/NKG2C receptor compared to binding of the TMMP to a CD94/NKG2A receptor.

16. The TMMP of any one of claims 1-14, wherein the NKG2 peptide comprises an amino acid sequence selected from the group consisting of QMPSRSLLF (SEQ ID NO://), TLPKRGLFL (SEQ ID NO://), TGPWRSLWI (SEQ ID NO://), ILTDRSLWL (SEQ ID NO://), VNPGRSLFL (SEQ ID NO://), VMAPRTLFL (SEQ ID NO://), TAPARTMFL (SEQ ID NO://), TLPERTLYL (SEQ ID NO://), VMPPRTLLL (SEQ ID NO://), VMPGRTLCF (SEQ ID NO://), RMPPRSVLL (SEQ ID NO://), NMPARTVLF (SEQ ID NO://), and VLPHRTQFL (SEQ ID NO://).

17. The TMMP of any one of claims 1-16, wherein:
a) the first polypeptide comprises, in order from N-terminus to C-terminus:
i) the NKG2 peptide;
ii) a peptide linker; and
iii) the β2M polypeptide; and
b) the second polypeptide comprises, in order from N-terminus to C-terminus
i) the HLA-E polypeptide;
ii) a peptide linker; and
iii) the Ig Fc polypeptide.

18. The TMMP of any one of claims 1-17, wherein the second polypeptide further comprises one or more immunomodulatory polypeptides, and wherein when the TMMP comprises more than one immunomodulatory polypeptide, one or more linkers may be interposed between the more than one immunomodulatory polypeptides.

19. The TMMP of claim 18, wherein at least one of the one or more immunomodulatory polypeptides is a wild-type or variant of an activating immunomodulatory polypeptide, optionally selected from IL-2, a 4-1BBL, CD80, CD86, and combinations thereof, and optionally wherein at least one immunomodulatory polypeptide is a variant immunomodulatory polypeptide that exhibits reduced affinity to a cognate costimulatory polypeptide compared to the affinity of a corresponding wild-type immunomodulatory polypeptide for the cognate costimulatory polypeptide.

20. The TMMP of claim 18, wherein the at least one immunomodulatory polypeptide is a variant of IL-2 that exhibits decreased binding affinity for IL-2Rα and IL-2Rβ, optionally wherein the variant IL-2 polypeptide comprises an amino acid other than histidine at position 16 and an amino acid other than phenylalanine at position 42.

21. The TMMP of claim 19, wherein the immunomodulatory polypeptide is a variant IL-2 polypeptide comprising an amino acid at position 16 other than His and an amino acid at position 42 other than Phe.

22. The TMMP of claim 21, wherein the at least one immunomodulatory polypeptide comprises i) an H16A substitution and an F42A substitution; ii) an H16T substitution and an F42A substitution, iii) an H16D and F42A substitution, or an H16E substitution and an F42A substitution, or iv) an H16E substitution and an F42A substitution.

23. The TMMP of any one of claims 18-22, wherein:
a) the first polypeptide comprises, in order from N-terminus to C-terminus:
i) the NKG2 peptide;
ii) a peptide linker; and
iii) the β2M polypeptide; and
b) the second polypeptide comprises, in order from N-terminus to C-terminus
i) the HLA-E polypeptide;
ii) a peptide linker;
iii) the Ig Fc polypeptide;
iv) a peptide linker; and
v) the one or more immunomodulatory polypeptides.

24. The TMMP of any one of claims 18-22, wherein:
a) the first polypeptide comprises, in order from N-terminus to C-terminus:
i) the NKG2 peptide;
ii) a peptide linker; and
iii) the β2M polypeptide; and
b) the second polypeptide comprises, in order from N-terminus to C-terminus
i) the one or more immunomodulatory polypeptides;
ii) a peptide linker;
iii) the HLA-E polypeptide;
iv) a peptide linker; and
v) the Ig Fc polypeptide.

25. The TMMP of any one of claims 1-17, wherein the second polypeptide further comprises a cancer-targeting polypeptide (CTP).

26. The TMMP of claim 25, wherein the CTP is an antibody specific for a cancer-associated antigen on the surface of a cancer cell.

27. The TMMP of claim 26, wherein the antibody is specific for Her2, CD19, WT1, MUC1, BCMA, mesothelin, TROP-2, PRAME, MAGE-A4, or a claudin polypeptide.

28. The TMMP of claim 26 or claim 27, wherein the antibody is a single-chain Fv (scFv) polypeptide.

29. The TMMP of claim 26 or claim 27, wherein the antibody is a nanobody.

30. The TMMP of any one of claims 25-29, wherein:
a) the first polypeptide comprises, in order from N-terminus to C-terminus:
i) the NKG2 peptide;
ii) a peptide linker; and
iii) the β2M polypeptide; and
b) the second polypeptide comprises, in order from N-terminus to C-terminus
i) the HLA-E polypeptide;
ii) a peptide linker;
iii) the Ig Fc polypeptide;
iv) a peptide linker; and
v) the CTP.

31. The TMMP of any one of claims 25-29, wherein:
a) the first polypeptide comprises, in order from N-terminus to C-terminus:
i) the NKG2 peptide;
ii) a peptide linker; and
iii) the β2M polypeptide; and
b) the second polypeptide comprises, in order from N-terminus to C-terminus
i) the CTP;
ii) a peptide linker;
iii) the HLA-E polypeptide;
iv) a peptide linker; and
v) the Ig Fc polypeptide;

32. The TMMP of any one of claims 25-31, wherein the second polypeptide further comprises one or more immunomodulatory polypeptides, and wherein when the TMMP comprises more than one immunomodulatory polypeptide, one or more linkers may be interposed between the more than one immunomodulatory polypeptides.

33. The TMMP of claim 32, wherein at least one of the one or more immunomodulatory polypeptides is a wild-type or variant of an activating immunomodulatory polypeptide, optionally selected from IL-2, a 4-1BBL, CD80, CD86, and combinations thereof, and optionally wherein at least one immunomodulatory polypeptide is a variant immunomodulatory polypeptide that exhibits reduced affinity to a cognate costimulatory polypeptide compared to the affinity of a corresponding wild-type immunomodulatory polypeptide for the cognate costimulatory polypeptide.

34. The TMMP of claim 33, wherein the at least one immunomodulatory polypeptide is a variant of IL-2 that exhibits decreased binding affinity for IL-2Rα and IL-2Rβ, optionally wherein the variant IL-2 polypeptide comprises an amino acid other than histidine at position 16 and an amino acid other than phenylalanine at position 42.

35. The TMMP of claim 34, wherein the immunomodulatory polypeptide is a variant IL-2 polypeptide comprising an amino acid at position 16 other than His and an amino acid at position 42 other than Phe.

36. The TMMP of claim 35, wherein the at least one immunomodulatory polypeptide comprises i) an H16A substitution and an F42A substitution; ii) an H16T substitution and an F42A substitution, iii) an H16D and F42A substitution, or an H16E substitution and an F42A substitution, or iv) an H16E substitution and an F42A substitution.

37. The TMMP of any one of claims 32-36, wherein:
a) the first polypeptide comprises, in order from N-terminus to C-terminus:
i) the NKG2 peptide;
ii) a peptide linker; and
iii) the β2M polypeptide; and
b) the second polypeptide comprises, in order from N-terminus to C-terminus
i) the HLA-E polypeptide;
ii) a peptide linker;
iii) the Ig Fc polypeptide;
iv) a peptide linker;
v) the one or more immunomodulatory polypeptides;
vi) a peptide linker; and
vii) the CTP.

38. The TMMP of any one of claims 18-22, wherein:
a) the first polypeptide comprises, in order from N-terminus to C-terminus:
i) the NKG2 peptide;
ii) a peptide linker; and
iii) the β2M polypeptide; and
b) the second polypeptide comprises, in order from N-terminus to C-terminus
i) the one or more immunomodulatory polypeptides;
ii) a peptide linker;
iii) the HLA-E polypeptide;
iv) a peptide linker;
v) the Ig Fc polypeptide;
vi) a peptide linker; and
vii) the CTP.

39. A homodimer comprising two TMMPs of any one of claims 1-38.

40. A nucleic acid comprising a nucleotide sequence encoding the first and the second polypeptides of any one of claims 1-38.

41. A recombinant expression vector comprising the nucleic acid of claim 40.

42. An in vitro host cell genetically modified with the recombinant expression vector of claim 41.

43. A plurality of nucleic acids, the plurality comprising:
a) a first nucleic acid comprising a nucleotide sequence encoding the first polypeptide of any one of claims 1-38; and
b) a second nucleic acid comprising a nucleotide sequence encoding the second polypeptide of any one of claims 1-38.

44. A plurality of recombinant expression vectors, the plurality comprising:
a) a first recombinant expression vector comprising the first nucleic acid of claim 43; and
b) a second recombinant expression comprising the second nucleic acid of claim 43.

45. An in vitro host cell genetically modified with the plurality of recombinant expression vectors of claim 44.

46. A method of producing a TMMP of any one of claims 1-38, the method comprising culturing the host cell of claim 44 or claim 47 in a culture medium, wherein the host cell produces the TMMP.

47. A pharmaceutical composition comprising the TMMP of any one of claims 1-38.

48. A method of treating cancer in an individual, the method comprising administering to the individual an effective amount of the TMMP of any one of claims 1-38, or the composition of claim wherein the TMMP selectively activates an NKG2C⁺ NK cell in the individual.

49. A method of treating an autoimmune disorder in an individual, the method comprising administering to the individual an effective amount of the TMMP of any one of claims 1-38, or the composition of claim 47, wherein the TMMP selectively inhibits an NKG2A⁺ NK cell in the individual.


SEQUENCE LISTING (153 sequences)

SEQ ID NO: 1 - protein, 275 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 2 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 3 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGAYNQSEAGSHTLQWMHGCELGPDRRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 4 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 5 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGAYNQSEAGSHTLQWMHGCELGPDRRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 6 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGCYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 7 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGCYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 8 - protein, 275 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 9 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 10 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGAYNQSEAGSHTLQWMHGCELGPDGRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 11 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 12 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGAYNQSEAGSHTLQWMHGCELGPDGRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 13 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGCYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 14 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGCYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 15 - protein, 99 aa, Homo sapiens [sequence omitted]

SEQ ID NO: 16 - protein, 99 aa, synthetic construct
IQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 17 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 227 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 226 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 29 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 30 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 31 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 32 - protein, 133 aa, Homo sapiens [sequence omitted]

SEQ ID NO: 33 - protein, 114 aa, synthetic construct
GCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 34 - protein, 123 aa, synthetic construct
QMPSRSLLFGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 35 - protein, 123 aa, synthetic construct
TLPKRGLFLGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 36 - protein, 123 aa, synthetic construct
TGPWRSLWIGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 37 - protein, 123 aa, synthetic construct
ILTDRSLWLGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 38 - protein, 123 aa, synthetic construct
VNPGRSLFLGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 39 - protein, 123 aa, synthetic construct
TAPARTMFLGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 40 - protein, 123 aa, synthetic construct
TLPERTLYLGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 41 - protein, 123 aa, synthetic construct
VMPPRTLLLGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 42 - protein, 123 aa, synthetic construct
VMPGRTLCFGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 43 - protein, 123 aa, synthetic construct
RMPPRSVLLGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 44 - protein, 123 aa, synthetic construct
NMPARTVLFGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 45 - protein, 123 aa, synthetic construct
VLPHRTQFLGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 46 - protein, 123 aa, synthetic construct
VMAPRTLILGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 47 - protein, 424 aa, synthetic construct
VMAPRTLILGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 48 - protein, 506 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGCYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWKAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 49 - protein, 817 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGCYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWKGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 807 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGCYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWKAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 51 - protein, 506 aa, synthetic construct; identical to SEQ ID NO: 48

SEQ ID NO: 52 - protein, 817 aa, synthetic construct; identical to SEQ ID NO: 49

SEQ ID NO: 53 - protein, 807 aa, synthetic construct; identical to SEQ ID NO: 50

SEQ ID NO: 54 - protein, 807 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGCYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWKAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 424 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSVMAPRTLILGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 56 - protein, 9 aa, synthetic construct
VMAPRTLLL

SEQ ID NO: 57 - protein, 9 aa, synthetic construct
VMAPRALLL

SEQ ID NO: 58 - protein, 9 aa, synthetic construct
VMAPRTVLL

SEQ ID NO: 59 - protein, 9 aa, synthetic construct
VMAPRTLVL

SEQ ID NO: 60 - protein, 9 aa, synthetic construct
QMPSRSLLF

SEQ ID NO: 61 - protein, 9 aa, synthetic construct
TLPKRGLFL

SEQ ID NO: 62 - protein, 9 aa, synthetic construct
TGPWRSLWI

SEQ ID NO: 63 - protein, 9 aa, synthetic construct
ILTDRSLWL

SEQ ID NO: 64 - protein, 9 aa, synthetic construct
VNPGRSLFL

SEQ ID NO: 65 - protein, 9 aa, synthetic construct
VMAPRTLFL

SEQ ID NO: 66 - protein, 9 aa, synthetic construct
TAPARTMFL

SEQ ID NO: 67 - protein, 9 aa, synthetic construct
TLPERTLYL

SEQ ID NO: 68 - protein, 9 aa, synthetic construct
VMPPRTLLL

SEQ ID NO: 69 - protein, 9 aa, synthetic construct
VMPGRTLCF

SEQ ID NO: 70 - protein, 9 aa, synthetic construct
RMPPRSVLL

SEQ ID NO: 71 - protein, 9 aa, synthetic construct
NMPARTVLF

SEQ ID NO: 72 - protein, 9 aa, synthetic construct
VLPHRTQFL

SEQ ID NO: 73 - protein, 9 aa, synthetic construct
QMPSRSLLF

SEQ ID NO: 74 - protein, 9 aa, synthetic construct
TLPKRGLFL

SEQ ID NO: 75 - protein, 9 aa, synthetic construct
TGPWRSLWI

SEQ ID NO: 76 - protein, 9 aa, synthetic construct
ILTDRSLWL

SEQ ID NO: 77 - protein, 9 aa, synthetic construct
VNPGRSLFL

SEQ ID NO: 78 - protein, 9 aa, synthetic construct
VMAPRTLFL

SEQ ID NO: 79 - protein, 9 aa, synthetic construct
TAPARTMFL

SEQ ID NO: 80 - protein, 9 aa, synthetic construct
TLPERTLYL

SEQ ID NO: 81 - protein, 9 aa, synthetic construct
VMPPRTLLL

SEQ ID NO: 82 - protein, 9 aa, synthetic construct
VMPGRTLCF

SEQ ID NO: 83 - protein, 9 aa, synthetic construct
RMPPRSVLL

SEQ ID NO: 84 - protein, 9 aa, synthetic construct
NMPARTVLF

SEQ ID NO: 85 - protein, 9 aa, synthetic construct
VLPHRTQFL

SEQ ID NO: 86 - protein, 9 aa, synthetic construct
WNRLFPPLR

SEQ ID NO: 87 - protein, 9 aa, synthetic construct
VMGDRSVLY

SEQ ID NO: 88 - protein, 9 aa, synthetic construct
VMADKSIFY

SEQ ID NO: 89 - protein, 9 aa, synthetic construct
TGAARSFFF

SEQ ID NO: 90 - protein, 9 aa, synthetic construct
VMAPRTLFL

SEQ ID NO: 91 - protein, 9 aa, synthetic construct
VMAPRTLIL

SEQ ID NO: 92 - protein, 133 aa, synthetic construct
  VARIANT 16: note = where X is an amino acid other than His
  VARIANT 42: note = where X is an amino acid other than Phe
APTSSSTKKTQLQLEXLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 93 - protein, 133 aa, synthetic construct; identical to SEQ ID NO: 92
  VARIANT 16: note = where X is Ala, Arg, Asn, Asp, Cys, Gln, Glu, Gly, Ile, Leu, Lys, Met, Phe, Pro, Ser, Thr, Trp, Tyr, or Val
  VARIANT 42: note = where X is Ala, Arg, Asn, Asp, Cys, Gln, Glu, Gly, His, Ile, Leu, Lys, Met, Pro, Ser, Thr, Trp, Tyr, or Val

SEQ ID NO: 94 - protein, 133 aa, synthetic construct
  VARIANT 16: note = where X is Ala, Arg, Asn, Asp, Cys, Gln, Glu, Gly, Ile, Leu, Lys, Met, Phe, Pro, Ser, Thr, Trp, Tyr, or Val
  VARIANT 38: note = where X is any amino acid other than Arg
  VARIANT 42: note = where X is Ala, Arg, Asn, Asp, Cys, Gln, Glu, Gly, His, Ile, Leu, Lys, Met, Pro, Ser, Thr, Trp, Tyr, or Val
APTSSSTKKTQLQLEXLLLDLQMILNGINNYKNPKLTXMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 95 - protein, 133 aa, synthetic construct
  VARIANT 42: note = where X is any amino acid other than Phe
  VARIANT 88: note = where X is any amino acid other than Asn
APTSSSTKKTQLQLEHLLLDLQMILNGINNYKNPKLTRMLTXKFYMPKKATELKHLQCLEEELKPLEEVLNLAQSKNFHLRPRDLISXINVIVLELKGSETTFMCEYADETATIVEFLNRWITFCQSIISTLT

SEQ ID NO: 96 - protein, 133 aa, synthetic construct
  VARIANT 38: note = where X is any amino acid other than Arg
  VARIANT 42: note = where X is any amino acid other than Phe
  VARIANT 88: note = where X is any amino acid other than Asn
APTSSSTKKTQLQLEHLLLDLQMILNGINNYKNPKLTXMLTXKFYMPKKATELKHLQCLEEELKPLEEVLNLAQSKNFHLRPRDLISXINVIVLELKGSETTFMCEYADETATIVEFLNRWITFCQSIISTLT

SEQ ID NO: 97 - protein, 133 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 98 - protein, 133 aa, synthetic construct; identical to SEQ ID NO: 97

SEQ ID NO: 99 - protein, 133 aa, synthetic construct
APTSSSTKKTQLQLETLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 100 - protein, 219 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 101 - protein, 167 aa, Homo sapiens
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 102 - protein, 240 aa, Homo sapiens
QEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSIL

SEQ ID NO: 103 - protein, 134 aa, Homo sapiens
LQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 104 - protein, 201 aa, Homo sapiens
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPT

SEQ ID NO: 105 - protein, 208 aa, Homo sapiens [sequence omitted]

SEQ ID NO: 106 - protein, 224 aa, Homo sapiens
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 107 - protein, 179 aa, Homo sapiens
QLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 108 - protein, 5 aa, synthetic construct
  VARIANT >5: note = the entire sequence of amino acids 1-5 may be repeated one or more times
GSGGS

SEQ ID NO: 109 - protein, 5 aa, synthetic construct
  VARIANT >5: note = the entire sequence of amino acids 1-5 may be repeated one or more times
GGGGS

SEQ ID NO: 110 - protein, 4 aa, synthetic construct
  VARIANT >4: note = the entire sequence of amino acids 1-4 may be repeated one or more times
GGGS

SEQ ID NO: 111 - protein, 4 aa, synthetic construct
GGSG

SEQ ID NO: 112 - protein, 5 aa, synthetic construct
GGSGG

SEQ ID NO: 113 - protein, 5 aa, synthetic construct
GSGSG

SEQ ID NO: 114 - protein, 5 aa, synthetic construct
GSGGG

SEQ ID NO: 115 - protein, 5 aa, synthetic construct
GGGSG

SEQ ID NO: 116 - protein, 5 aa, synthetic construct
GSSSG

SEQ ID NO: 117 - protein, 5 aa, synthetic construct
AAAGG

SEQ ID NO: 118 - protein, 100 aa, synthetic construct
  VARIANT 6..100: note = wherein up to 19 copies of EAAAK may be deleted
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 119 - protein, 101 aa, synthetic construct
  VARIANT 7..101: note = wherein up to 19 copies of EAAAK may be deleted
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 120 - protein, 102 aa, synthetic construct
  VARIANT 7..101: note = wherein up to 19 copies of EAAAK may be deleted
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 121 - protein, 206 aa, synthetic construct
  VARIANT 7..101: note = wherein up to 19 copies of EAAAK may be deleted
  VARIANT 106..205: note = wherein up to 19 copies of EAAAK may be deleted
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKALEAEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 122 - protein, 40 aa, synthetic construct
  VARIANT 3..40: note = wherein up to 19 copies of KP may be deleted
KPKPKPKPKPKPKPKPKPKPKPKPKPKPKPKPKPKPKPKP

SEQ ID NO: 123 - protein, 40 aa, synthetic construct
  VARIANT 3..40: note = wherein up to 19 copies of EP may be deleted
EPEPEPEPEPEPEPEPEPEPEPEPEPEPEPEPEPEPEPEP

SEQ ID NO: 124 - protein, 60 aa, synthetic construct
  VARIANT 4..60: note = wherein up to 19 copies of TPR may be deleted
TPRTPRTPRTPRTPRTPRTPRTPRTPRTPRTPRTPRTPRTPRTPRTPRTPRTPRTPRTPR

SEQ ID NO: 125 - protein, 40 aa, synthetic construct
  VARIANT 3..40: note = wherein up to 19 copies of AP may be deleted
APAPAPAPAPAPAPAPAPAPAPAPAPAPAPAPAPAPAPAP

SEQ ID NO: 126 - protein, 55 aa, synthetic construct
  VARIANT 11..55: note = wherein up to 9 copies of GGGGS may be deleted
CGGGSGGGGSGGGGSGGGGSGGGGSGGGGSGGGGSGGGGSGGGGSGGGGSGGGGS

SEQ ID NO: 127 - protein, 55 aa, synthetic construct
  VARIANT 11..55: note = wherein up to 9 copies of GGGGS may be deleted
GCGGSGGGGSGGGGSGGGGSGGGGSGGGGSGGGGSGGGGSGGGGSGGGGSGGGGS

SEQ ID NO: 128 - protein, 55 aa, synthetic construct
  VARIANT 11..55: note = wherein up to 9 copies of GGGGS may be deleted
GGCGSGGGGSGGGGSGGGGSGGGGSGGGGSGGGGSGGGGSGGGGSGGGGSGGGGS

SEQ ID NO: 129 - protein, 5 aa, synthetic construct
EAAAK

SEQ ID NO: 130 - protein, 10 aa, synthetic construct
EAAAKEAAAK

SEQ ID NO: 131 - protein, 15 aa, synthetic construct
EAAAKEAAAKEAAAK

SEQ ID NO: 132 - protein, 46 aa, synthetic construct
AEAAAKEAAAKEAAAKEAAAKALEAEAAAKEAAAKEAAAKEAAAKA

SEQ ID NO: 133 - protein, 12 aa, synthetic construct
AEAAAKEAAAKA

SEQ ID NO: 134 - protein, 5 aa, synthetic construct
PAPAP

SEQ ID NO: 135 - protein, 8 aa, synthetic construct
APAPAPAP

SEQ ID NO: 136 - protein, 12 aa, synthetic construct
APAPAPAPAPAP

SEQ ID NO: 137 - protein, 16 aa, synthetic construct
APAPAPAPAPAPAPAP

SEQ ID NO: 138 - protein, 20 aa, synthetic construct
APAPAPAPAPAPAPAPAPAP

SEQ ID NO: 139 - protein, 4 aa, synthetic construct
KPKP

SEQ ID NO: 140 - protein, 8 aa, synthetic construct
KPKPKPKP

SEQ ID NO: 141 - protein, 12 aa, synthetic construct
KPKPKPKPKPKP

SEQ ID NO: 142 - protein, 16 aa, synthetic construct
KPKPKPKPKPKPKPKP

SEQ ID NO: 143 - protein, 20 aa, synthetic construct
KPKPKPKPKPKPKPKPKPKP

SEQ ID NO: 144 - protein, 4 aa, synthetic construct
EPEP

SEQ ID NO: 145 - protein, 8 aa, synthetic construct
EPEPEPEP

SEQ ID NO: 146 - protein, 12 aa, synthetic construct
EPEPEPEPEPEP

SEQ ID NO: 147 - protein, 16 aa, synthetic construct
EPEPEPEPEPEPEPEP

SEQ ID NO: 148 - protein, 20 aa, synthetic construct
EPEPEPEPEPEPEPEPEPEP

SEQ ID NO: 149 - protein, 5 aa, synthetic construct
GGGGS

SEQ ID NO: 150 - protein, 10 aa, synthetic construct
GCGGSGGGGS

SEQ ID NO: 151 - protein, 15 aa, synthetic construct
GCGGSGGGGSGGGGS

SEQ ID NO: 152 - protein, 15 aa, synthetic construct
GGGGSGGGGSGGGGS

SEQ ID NO: 153 - protein, 20 aa, synthetic construct
GGGGSGGGGSGGGGSGGGGS